SEQUENCE LISTING (92 sequences)

SEQ ID NO: 1 - DNA, 1533 bp, Populus tremula x alba
atgtcagtcgctaacccagagccacatcgagtcagcaggtctcgacaaaccaaccgacccccatcaactcgacaccaccaacaacaatcaagcagggttccgttacgccaactcctccgcgtgacatcaatcgctggtgggatacaattcggctgggcgttacagctctctctcttaacaccctacgtgcaggagctaggaatccctcacaaatgggccagtataatctggttgtgcggcccattatcgggattgtttgtgcaacctcttgttggcgtcatgagcgatcggtgcaccagccggtttggaaggaggaggccgtttatttttgcgggttcgcttttgattgcgatatcggttttgattatcggtcattccgctgacattgggtggtggttaggcgatagaggaggtgttagaccaagagcaattggcgcttttgtgtttgggttttggattctcgatgttgctaataatatgacccagggtccttgtcgtgctcttctcgctgatcttactggaaaagatcatcgaagaactagagtcgcaaatgcttatttctcacttttcatggctgttggtaatattcttggttttgccactggatcatataatgggtggtacaaagtttttccttttactgttacttctgcatgcaatattgactgtgcgaatctcaagtctgccttttatctcgatgttgttttcatggcaattactgcctgcataagcatcgcagcagctcaagaatcacctctagatttacctgccagatctatgctcgctgatgaggaaacgccaggccattcaaactctgaacaagaggctttcatctgggagctatttgggacttttagatgtttcccatcaactgtatggataatattgcttgttactgctctgaattggattggttggtttccatttctgctcttcgacactgactggatgggtcgagaaatttatggtggcaagccaaatgaaggacagaattataacactggagtcagaatgggtgcatttggtctgatgttcaattcagtaattcttggagtgacatctgtgctcatggagaagctctgcagcaagtggggagcaggtttcttatggggcctttcgaacatcttaatggctctctgctttctttcaatgcttgtactctcctatgtggcaagccatattggctacatgggacataatctaccaccagatagcattgtagtaattgcactagtgatttttgcagtgcttggcatgccattggcgatcacttacagtgttccatatgctatggtttcctcacgtattgaatccttgggacttggacaagggttgtcaatgggtgttcttaatctggcaattgtgcttccacaggtggtggtttctgtcggaagtggaccatgggatcaaatatttggtggtggaaactccccagctattgctattggagcacttgctgcctttgctgctggaattatagccatcttgggtattcgtcgatcaggagttcaaaagcccatggtcttctcatga

SEQ ID NO: 2 - protein, 510 aa, Populus tremula x alba
MSVANPEPHRVSRSRQTNRPPSTRHHQQQSSRVPLRQLLRVTSIAGGIQFGWALQLSLLTPYVQELGIPHKWASIIWLCGPLSGLFVQPLVGVMSDRCTSRFGRRRPFIFAGSLLIAISVLIIGHSADIGWWLGDRGGVRPRAIGAFVFGFWILDVANNMTQGPCRALLADLTGKDHRRTRVANAYFSLFMAVGNILGFATGSYNGWYKVFPFTVTSACNIDCANLKSAFYLDVVFMAITACISIAAAQESPLDLPARSMLADEETPGHSNSEQEAFIWELFGTFRCFPSTVWIILLVTALNWIGWFPFLLFDTDWMGREIYGGKPNEGQNYNTGVRMGAFGLMFNSVILGVTSVLMEKLCSKWGAGFLWGLSNILMALCFLSMLVLSYVASHIGYMGHNLPPDSIVVIALVIFAVLGMPLAITYSVPYAMVSSRIESLGLGQGLSMGVLNLAIVLPQVVVSVGSGPWDQIFGGGNSPAIAIGALAAFAAGIIAILGIRRSGVQKPMVFS

SEQ ID NO: 3 - DNA, 1533 bp, Arabidopsis thaliana
atggctacttccgatcaagatcgccgtcacagagtcactcgcaaccgtccaccaatagctcgaccctctacttcttcatctcgtcccgttgtatctcctcctagatcaaaagtttcgaagcgtgtgcttctccgtgtagcttccgtcgcatgcgggattcaattcggatgggctcttcagctttctctcctcacaccttacgttcaagagctggggatcccacacgcttgggctagtgtgatttggctttgcggtcctctctctggtttgttcgtgcaaccgctcgttgggcatagtagcgataggtgtactagtaagtacggtcgtcggagaccgtttattgtcgccggagctgtggcgatttctatctctgttatggttattggtcatgcggcggatattggatgggcatttggggatagagaagggaagattaagccgagggcgattgttgcttttgttttggggttttggattcttgatgttgctaataatatgactcaaggtccttgtagagctctccttgctgatcttactgagaatgataatcgcagaacccgagttgcaaatggctacttctctctctttatggctgttggcaatgttcttggctatgctactggatcatacaatggttggtacaagatcttcacttttacgaagacagttgcatgtaatgtggaatgtgccaatctcaagtctgccttctacatagatgttgtctttattgcaataactacgatcctaagcgtctcagcggctcatgaggtgccacttgcttcattggcttctgaagcacatgggcaaaccagtggaacagacgaagcttttctttctgagatatttggaactttcagatattttccaggaaatgtttggataatcttgcttgttacagcattgacatggattggttggtttccatttattctgtttgatactgattggatgggtcgagagatctatggcggtgaaccgaacatagggacttcatatagtgctggggtcagtatgggtgcacttggtttgatgttgaattctgtttttcttggaatcacttctgtgctcatggagaaactttgcagaaagtggggggctggttttgtttggggaatatcaaatatcttaatggctatttgctttcttggaatgataatcacctcatttgttgcgtctcaccttggctacattggccatgaacaacctcctgccagcatcgtgtttgctgctgtgttaatctttacaattctgggcattccattggcgataacttacagcgtcccatatgcgttgatttccatacgtattgaatccctgggcttaggtcaaggcttatctttgggtgtgctaaatttggcgatagtcatcccacaggtaattgtgtctgttggcagtggcccatgggatcaactgtttggaggtgggaattcaccggcacttgcagtaggagcagctacaggcttcattggcggaattgtagctatcttggctcttccacggacaaggattcagaagcccatccctctcccatga

SEQ ID NO: 4 - protein, 510 aa, Arabidopsis thaliana
MATSDQDRRHRVTRNRPPIARPSTSSSRPVVSPPRSKVSKRVLLRVASVACGIQFGWALQLSLLTPYVQELGIPHAWASVIWLCGPLSGLFVQPLVGHSSDRCTSKYGRRRPFIVAGAVAISISVMVIGHAADIGWAFGDREGKIKPRAIVAFVLGFWILDVANNMTQGPCRALLADLTENDNRRTRVANGYFSLFMAVGNVLGYATGSYNGWYKIFTFTKTVACNVECANLKSAFYIDVVFIAITTILSVSAAHEVPLASLASEAHGQTSGTDEAFLSEIFGTFRYFPGNVWIILLVTALTWIGWFPFILFDTDWMGREIYGGEPNIGTSYSAGVSMGALGLMLNSVFLGITSVLMEKLCRKWGAGFVWGISNILMAICFLGMIITSFVASHLGYIGHEQPPASIVFAAVLIFTILGIPLAITYSVPYALISIRIESLGLGQGLSLGVLNLAIVIPQVIVSVGSGPWDQLFGGGNSPALAVGAATGFIGGIVAILALPRTRIQKPIPLP

SEQ ID NO: 5 - DNA, 1506 bp, Vitis vinifera
atggcggtccctgggggccatcggcagcgaggcaggcctcgagctctgatcggggaaccagtccgacccagggttccactgaggcggttgctgagagtggcatcggtggcctgtggaatacagtttggttgggctctgcagctctcccttctcacgccctacgtgcaagagctcggaattcctcacgcctggtccagtatcatatggctttgcggtcccctctcgggtctgctggtccagcccctcgtcggacacctcagtgatcgctgtaacagccgctttggccgccggaggcctttcatcgtcgccggagctacgtcgattgtcgtcgccgttctgataatcggattctcggctgacatcgggggtttgcttggtgacggtgccgaccggaggccgagggcggtggctacctttgtggtagggttttggttattggatgtggctaataacgtgactcaaggaccttgtagagctctgctagcagatctgaccgaaaaggatcatcgaaggacaagagtggccaacgcgtatttctcactattcattgctgtcggcaatgtccttggctttgctaccggatcctacagtggttggttcaggattttctggttcacgagcacctcttcatgtaatgctgattgtgccaatctcaagtctgcctttttactagacattatttttattgctatcaccacatatataagcataacagcagcccaggaattgcctctaagttccagcagtcgatccactcacatttcagaagaaatggcagagtcaacccatgctcaagaagcttttctttgggaactatttgggactcttagatatctctcagggtctatttggataatcctgtttgtaactgctttgacttggattggattgcttccttttcttctttttgatactgattggatgggtcgagaaatttatggtggcaaaccaaatgaagggcagaattataatactggagttagaatgggtgcgctcggtctgatgttgaattcagttgttcttggaataacttcagtgctcatggagaagctctgcaggaagtggggggctggttttgtttgggggctatcaaatattctcatgtctctttgctttctattaatgctcatactttctgctgtggtgaaacatatggattttctaggtcatgatctacccccaagtggtgttgtgattgctgcactgattgttttttcaatccttggtattccattggcaatcacatatagtgttccatatgccttgatatccacacgaatcgagtccctgggactaggtcaagggttatcaatgggtgttctgaatttggcaattgtaattccccaggttattgtgtccctaggaagtggaccatgggatcaactttttggtggtggcaactcgccatccttggctgtggcagcagttgcagcattcgccagtggacttgtagccatcttggctattcctcgatctagtgctgataaatccagggtccacacatga

SEQ ID NO: 6 - protein, 501 aa, Vitis vinifera
MAVPGGHRQRGRPRALIGEPVRPRVPLRRLLRVASVACGIQFGWALQLSLLTPYVQELGIPHAWSSIIWLCGPLSGLLVQPLVGHLSDRCNSRFGRRRPFIVAGATSIVVAVLIIGFSADIGGLLGDGADRRPRAVATFVVGFWLLDVANNVTQGPCRALLADLTEKDHRRTRVANAYFSLFIAVGNVLGFATGSYSGWFRIFWFTSTSSCNADCANLKSAFLLDIIFIAITTYISITAAQELPLSSSSRSTHISEEMAESTHAQEAFLWELFGTLRYLSGSIWIILFVTALTWIGLLPFLLFDTDWMGREIYGGKPNEGQNYNTGVRMGALGLMLNSVVLGITSVLMEKLCRKWGAGFVWGLSNILMSLCFLLMLILSAVVKHMDFLGHDLPPSGVVIAALIVFSILGIPLAITYSVPYALISTRIESLGLGQGLSMGVLNLAIVIPQVIVSLGSGPWDQLFGGGNSPSLAVAAVAAFASGLVAILAIPRSSADKSRVHT

SEQ ID NO: 7 - DNA, 1503 bp, Solanum lycopersicum [Lycopersion esculentum]
atgccggagatagaaaggcatagaacaaggcataaccgaccggcgattcgagaaccggtgaaaccgagagtaccactgagactattgttacgagtagcttcggttgccggtggaattcaattcggttgggcgttacaactatcgctgctcacaccttatgtgcaggagcttggaataccgcatgcttgggcgagcataatatggctctgtggaccgctttcaggtttattggttcagcctttagtaggtcacatgagtgacaagtgcacaagtcggttcggtcgtcggcgtccgtttattgtcgccggagcagcatcaatcatgattgcggtgttgattatcggtttctccgctgatattggatggcttttaggtgatcgaggtgaaataaaagtgcgtgctatagcggcgtttgttgtagggttttggcttctcgatgttgctaataatatgactcaaggaccttgcagagctctgcttgctgatcttacacaaaaggatcacagaagaactcgggtagcaaatgcatatttttccttatttatggccattggtaacatccttggctttgctactggatcttacagtggctggtacaagatcttcctttttactctcaatactgcatgcactatcaactgtgccaatctaaaggctgcttttattctcgacattatttttattgcaacaacaacatgcattagcatatcggcggccaatgagcagcctctagatcccagtcgtggttcctctcatactggagaagagattgacgaatcaagccatggtcaagaagaagcttttctctgggagttgtttggtattttcaagtatttcccaggtgttgtttgggtgatcctgcttgtcactgccctgacatggattggatggtttccatttcttttgttcgatactgactggtttggtcgagaaatttatggcggtgaaccaaatgacggaaagaattatagtgcaggagtgcggatgggttcattgggtctaatgttgaattctgtccttcttggactaacttcattgttcatggagaagctctgtcgaaaatggggggctggtttcacatggggagtttctaacgtggtcatgtctctctgctttatagccatgcttataattactgctgttaggagtaacatagacattggccagggtcttccgccggatggaattgtgattgctgcgctggttgtattttctattcttgggatcccactagctataacgtacagtgttccatatgctttagtatcctcaaggattgaagctcttgggcttggacaaggcttgtcaatgggcgtgctgaacctggcaattgtgttcccacagattgtggtttcactgggaagtgggccatgggatgagctatttggtggaggcaattcaccagcctttgttgtggctgcgctttcagcatttgctgctggacttatagccatcttggcaattcctcgaacacgggttgagagacccaagatctttgcatga

SEQ ID NO: 8 - protein, 500 aa, Solanum lycopersicum [Lycopersion esculentum]
MPEIERHRTRHNRPAIREPVKPRVPLRLLLRVASVAGGIQFGWALQLSLLTPYVQELGIPHAWASIIWLCGPLSGLLVQPLVGHMSDKCTSRFGRRRPFIVAGAASIMIAVLIIGFSADIGWLLGDRGEIKVRAIAAFVVGFWLLDVANNMTQGPCRALLADLTQKDHRRTRVANAYFSLFMAIGNILGFATGSYSGWYKIFLFTLNTACTINCANLKAAFILDIIFIATTTCISISAANEQPLDPSRGSSHTGEEIDESSHGQEEAFLWELFGIFKYFPGVVWVILLVTALTWIGWFPFLLFDTDWFGREIYGGEPNDGKNYSAGVRMGSLGLMLNSVLLGLTSLFMEKLCRKWGAGFTWGVSNVVMSLCFIAMLIITAVRSNIDIGQGLPPDGIVIAALVVFSILGIPLAITYSVPYALVSSRIEALGLGQGLSMGVLNLAIVFPQIVVSLGSGPWDELFGGGNSPAFVVAALSAFAAGLIAILAIPRTRVERPKIFA

SEQ ID NO: 9 - DNA, 1503 bp, Solanum tuberosum
atgccggagatagaaaggcatagaacaaggcataaccgaccggcgattcgagaaccggtgaaaccgagagtaccactgagactattgttccgagtagcttcggttgccggtggaattcaattcggttgggcgttacaactatcactgctcacaccttatgtgcaagagcttggaataccgcatgcttgggcgagcataatatggctctgtggaccgctttcaggtttactggttcagcctttagtaggtcacatgagtgacaagtgcacaagtcggttcggtcgtcggcgcccgtttattgtcgccggagcagtatcgatcatgattgcggtgttgattatcggtttctccgctgatattggatggcttttaggtgatcgaggtgaaataaaagtgcgtgctatagcggcgtttgtcgtagggttttggcttctcgatgttgccaataatatgactcaaggaccttgcagagctctgcttgctgatcttacacaaaaggatcatagaagaacccgggtagcaaatgcatatttttccttatttatggccattggtaacatccttggctttgctactggatcttacagtggctggttcaagatcttcccttttactctcaatactgcatgcaccatcaactgtgccaatctaaaggctgcttttattatcgacattatttttattgcaacaactacatgcattagcatatcagcggccaatgagcagcctctagatcccagtcgtggttcctctcataccagagaagagattggcgaatcaagccatggtcaagaagaagcttttctctgggagttgtttggaattttcaagtatttcccaggtgttgtttgggtgatcctgcttgtcactgccctgacatggattggatggtttccgtttcttttgttcgatactgactggtttggtcgagaaatttatggcggtgaaccaaatgatggaaagaattatagtgcaggagtgcgaatgggttcattgggtctaatgttgaattctgtgcttcttggactaacttcattgttcatggagaagctctgtcgaaaatggggtgctggtttcacatggggagtttcaaacgtggtcatgtctctctgttttatagccatgcttataattactgctgttaggagtaacatagacattggccagggtcttccaccggatggcattgtgattgctgcgctggttgtattttctattcttgggatcccactagctataacatacagtgttccatatgctttagtatcctcaaggattgatgctcttgggcttggacaaggcttgtcaatgggtgtgctgaacctggcaattgtgttcccacagattgtggtttctctgggaagtgggccatgggatgagttatttggtggaggcaattcaccagcctttgttgtggctgcgctttcagcatttgctggtggacttatagccatcttggcgattcctcgaacacgggttgagaaacccaagatctttgcatga

SEQ ID NO: 10 - protein, 500 aa, Solanum tuberosum
MPEIERHRTRHNRPAIREPVKPRVPLRLLFRVASVAGGIQFGWALQLSLLTPYVQELGIPHAWASIIWLCGPLSGLLVQPLVGHMSDKCTSRFGRRRPFIVAGAVSIMIAVLIIGFSADIGWLLGDRGEIKVRAIAAFVVGFWLLDVANNMTQGPCRALLADLTQKDHRRTRVANAYFSLFMAIGNILGFATGSYSGWFKIFPFTLNTACTINCANLKAAFIIDIIFIATTTCISISAANEQPLDPSRGSSHTREEIGESSHGQEEAFLWELFGIFKYFPGVVWVILLVTALTWIGWFPFLLFDTDWFGREIYGGEPNDGKNYSAGVRMGSLGLMLNSVLLGLTSLFMEKLCRKWGAGFTWGVSNVVMSLCFIAMLIITAVRSNIDIGQGLPPDGIVIAALVVFSILGIPLAITYSVPYALVSSRIDALGLGQGLSMGVLNLAIVFPQIVVSLGSGPWDELFGGGNSPAFVVAALSAFAGGLIAILAIPRTRVEKPKIFA

SEQ ID NO: 11 - DNA, 3406 bp, Populus trichocarpa
atggagagtggagttagaaaagaaaacaatcccccttcctcttctttcagtcttcaacaacaacctcctgcaacaaaccctagcccactgagaaagatcataatggttgcatcgatagctgctggtgttcaatttggttgggcgctacagctctctttgttaaccccatatgtgcaactcttgggcattcctcacacatgggctgctttcatctggctttgcggcccgatttctggcatgttagtccagccaacagtggggtactacagtgaccgatgcacctcccgttttggccgtcgtagcccgtttattgcggcgggggctggttttgttgccatttctgtgtttctcattggctatgccgctgacattggccatctctctggtgattccctcacaaaaacagctaagccacgcgcaattgctgtgtttgtggtagggttctggattcttgatgttgctaataacatgcttcaaggtccatgtagggcatttcttgctgatctttcagggacggatcacaagaagactcgtacagccaatgctttctactctttctttatggctgttggcaatgttcttggctttgcatctgggtcttacactcacttgtacagaatctttccattttcaagaaccaaggcttgtgatgtttactgtgcaaatctcaaatcttgtttcttcatctccattgccttgcttttaacgttgacaattctagctctctcttacgtacgtgaaaaaccctggtcgccggagggaagttccggtgacggaggtaacgaggaggagaaagaggtggagggtggagaagctaaagagtcgacgccagcgcctttttttggggaaatagtcgctgctcttaagaacttgcaaagacctatgaggatcctccttttggtgacgtgtctaaactgggttgcgtggtttcctttcttgttgtttgatactgattggatggggagagaggtgtacggtggtgattcaagtagaaatgctgatcaactgaagatgtatgatcgtggggtccatgcaggtgcattggggttactgcttaactcggtggttttgggattcacttcacttggtgtggaggttttggctcgtggggttggcggggttaaaaggctgtgggggattgtaaattttatcttggctatttgtttggctatgactattttgatcaccaaagtggctcaatctaacagaaggtacaccaccgtgaatggaggaacccacctcttgccgccgccatctggtgtcaaggctggtgctctagccctttttgctgttatgggcatacctcaagctgtaagttgttgttggcttgctttaattctcttcatgtattgttttgtgagtagtaattgaagtcagcctttttggtgcaggtcatctagtttttttttttttttgcatgctcaactgtatcatttttgtcttttactctccttttttgcctttttgttataaaattttattttgaatctggaattttccttttacgtttctttcttcacataaacgtttcctttccctttcccttttttccttttgttttaaattacttgacagttatgaattaaatttttaacataaacttgactgtaaaatcctgttaatcattggtataaattttttacataaacttgatgaagttgttaatcttgctgttgaaatatgtaattgggatttcatagtgtaattagataaggtgcacaacacccccttcggcggcaacagtgtaaatttttcaagcctctagtaacctgtctcctgctcgtgcatggagccacgtagtttaaaactggcaaattttcagagttttggttcctttcagatttcaatggccatccaatgcatgaacaccaaataataaatgcccccgtgggagtgaatgtgtgtttttttggctggtttaggcagcttatgattcttatctgatctttacaaaacttaatgaatgaaaaatcaagaccctttgttttcttctcccgtctccttttgttcttgatcaaccgcaagaaggtcaaacgtacgtgtagttcaacttgcacaagcacagccccctttatcaatgtatctgtgtgcatttaatgagaactggttgattggtagctgctgctgtaattgttgttggaaatgctgcagatgctccttttcgtttaaggattggagatttttcatgatgaactaatggtggcagttattatgcagataacttacagcatcccttttgctttggcatccatcttttctaacacttcgggtgctggccaaggtaagttttttagttccaagcagtacttctgctaaggtatcctcctaaattattaatttccttgtttcaatttgattatagggctttctcttggagttctcaatctttcaatagttataccacaggtcagtatatacacttcatgctaatacatgttgctgctataaattcactctagggattgtttttccatgcccgtttctgtttccaagcattaagcctccacaagtttgtgccttcattttattttgatgatggttgttttaagcaagtccttcagtactactggacaatgccacagtaatgatctgtgaatctggaaaggaaaaaaaaatattcaacaagtattttgcttctttaggatcaaaattgataaagaaatcatcagtccgatgataacctatcttcttctgtcatgattaaacggaatgtttgttagagaagcacactgggctacgatgtctgctgctcttggagttattgaacaggtctacaaacactttttcactagaaagcactaaagggaaagctttggattggattcgaagtatggatgcagtgctggcttctgaaattattgttaaaaaaggatgcataactttttatagacagaacaaagttctcttgactcatgatcatattctgtgaaccccaagaacgatagactgacattataaccataccattaaaaaattaaaaacaagaagaaaaagaagcgaatctcattcaattcaatgtagtgaagggagagaaaaaagaaagcaattaactctcagttatttaattgattggtgaccgccacatcttgaatttagtttacaaatgattagattagattcatatgtactgcagtatactagaatatgtgcaacagttaattctgaataaatgaatggtaattttaacttgattattgttttaatgatgttatggcagatggtggtttctgtagcagctggaccttgggatgccctttttggaggaggaaatctaccagcttttgttgtaggagcggttgcagctgcagccagtggaatattggcattcaccatgcttccatctccgccccctgacatcccctcaaacaagagggctgccaccagtagtagtgctgcattccattga

SEQ ID NO: 12 - DNA, 3406 bp, Populus trichocarpa
atggagagtggagttagaaaagaaaacaatcccccttcctcttctttcagtcttcaaaaacaacctcctgcaacaaaccctagcccactgagaaagatcataatggttgcatcgatagctgctggtgttcaatttggttgggcgctacagctctctttgttaaccccatatgtgcaactcttgggcattcctcacacatgggctgctttcatctggctttgcggcccgatttctggcatgttagtccagccaacagtggggtactacagtgaccgatgcacctcccgttttggccgtcgtagcccgtttattgcggcgggggctggttttgttgccatttctgtgtttctcattggctatgccgctgacattggccatctctctggtgattccctcacaaaaacagctaagccacgcgcaattgctgtgtttgtggtagggttctggattcttgatgttgctaataacatgcttcaaggtccatgtagggcatttcttgctgatctttcagggacggatcacaagaagactcgtacagccaatgctttctactctttctttatggctgttggcaatgttcttggctttgcatctgggtcttacactcacttgtacagaatctttccattttcaagaaccaaggcttgtgatgtttactgtgcaaatctcaaatcttgtttcttcatctccattgccttgcttttaacgttgacaattctagctctctcttacgtacgtgaaaaaccctggtcgccggagggaagttccggtgacggagctaacgaggaggagaaagaggtggagggtggagaagctaaagagtcgacgccagcgcctttttttggggaaatagtcgctgctcttaagaacttgcaaagacctatgaggatcctccttttggtgacgtgtctaaactgggttgcgtggtttcctttcttgttgtttgatactgattggatggggagagaggtgtacggtggtgattcaagtagaaatgctgatcaactgaggatgtatgatcgtggggtccgtgcaggtgcattggggttactgcttaactcggtggttttgggattcacttcacttggtgtggaggttttggctcgtggggttggcggggttaaaaggctgtgggggattgtaaattttatcttggctatttgtttggctatgactattttgatcaccaaagtggctcaatctaacagaaggtacaccaccgtgaatggaggaacccacctcttgccgccgccatctggtgtcaaggctggtgccctagccctttttgctgttatgggcatacctcaagctgtaagttgttgttggcttgctttaattctcttcatgtattgttttgtgagtagtaattgaagtcagcctttttggtgcaggtcatctagtttttttttttttttgcatgctcaactgtatcatttttgtcttttactctccttttttgcctttttgttataaaattttattttgaatctggaattttccttttacgtttctttcttcacataaacgtttcctttccctttcccttttttccttttgttttaaattacttgacagttatgaattaaatttttaacataaacttgactgtaaaatcctgttaatcattggtataaattttctacataaacttgatgaagttgttaatcttgctgttgaaatatgtaattgggatttcatagtgtaattagataaggtgcacaacacccccttcggcggcaacagtgtaaatttttcaagcctctagtaacctgtctcctgctcgtgcatggagccacgtagtttaaaactggcaaattttcagagttttggttcctttcagatttcaatggccatccaatgcatgaacaccaaataataaatgcccccgtgggagtgaatgtgtgtttttttggctggtttaggcagcttatgattcttatctgatctttacaaaacttaatgaatgaaaaatcaagaccctttgttttcttctcccgtctccttttgttcttgatcaaccgcaagaaggtcaaacgtacgtgtagttcaacttgcacaagcacagccccctttatcaatgtatctgtgtgcatttaatgagaactggttggttggtagctgctgctgtaattgttgttggaaatgctgcagatgctccttttcgtttaaggattggagatttttcatgatgaactaatggtggcagttattatgcagataacttacagcatcccttttgctttggcatccatcttttctaacacttcgggtgctggccaaggtaagttttttagttccaagcagtacttctgctaaggtatcctcctaaattattaatttccttgtttcaatttgattatagggctttctcttggagttctcaatctttcaatagttataccacaggtcagtatatacacttcatgctaatacatgttgctgctataaattcactctagggattgtttttccatgcccgtttctgtttccaagcattaagcctccacaagtttgtgccttcattttattttgatgatggttgttttaagcaagtccttcagtactactggacaatgccacagtaatgatctgtgaatctggaaaggaaaaaaaaatattcaacaagtattttgcttctttaggatcaaaattgataaagaaatcatcagtccgatgataacctatcttcttctgtcatgattaaacggaatgtttgttagagaagcacactgggctacgatgtctgctgctcttggagttattgaacaggtctacaaacactttttcactagaaagcactaaagggaaagctttggattggattcgaagtacggatgcagtgctggcttctgaaattattgttaaaaaaggatgcataactttttatagacagaacaaagttctcttgactcatgatcatattctgtgaaccccaagaacgatagactacattataaccataccattaaaaaattaaaaacaagaagagaaaagaagcgaatctcattcaattcaatgtagtgaagggagagaaaaaagaaagcaattaactctcagttatttaattgattggtgaccgccacatcttgaatttagtttacaaatgattagattagattcatatgtactgcagtatactagaatatgtgcaacagttaattctgaataaatgaatggtaattttaacttgattattgttttaatgatgttatggcagatggtggtttctgtagcagctggaccttgggatgccctttttggaggaggaaatctaccagcttttgttgtaggagcggttgcagctgcagccagtggaatattggcattcaccatgcttccatctccgccccctgacatcccctcaaacaagagggctgccaccagtagtagtgctgcattccattga

SEQ ID NO: 13 - DNA, 3367 bp, Populus trichocarpa
  misc_feature (3277)..(3326): n is a, c, g, or t
atggagagtggagttagaaaagaaaacaatcccccttcctcttctttcagtcttcaacaacaacctcctgcaacaaaccctagcccactgagaaagatcataatggttgcatcgatagctgctggtgttcaatttggttgggcgctacagctctctttgttaaccccatatgtgcaactcttgggcattcctcacacatgggctgctttcatctggctttgcggcccgatttctggcatgttagtccagccaacagtggggtactacagtgaccgatgcacctcccgttttggccgtcgtcgcccgtttattgcggcgggggctggttttgttgccatttctgtgtttctcattggctatgccgctgacattggccatctctctggtgattccctcacaaaaacagctaagccacgcgcaattgctgtgtttgtggtagggttctggattcttgatgttgctaataacatgcttcaaggtccatgtagggcatttcttgctgatctttcagggacggatcacaagaagactcgtacagccaatgctttctactctttctttatggctgttggcaatgttcttggctttgcatctgggtcttacactcacttgtacagaatctttccattttcaagaaccaaggcttgtgatgtttactgtgcaaatctcaaatcttgtttcttcatctccatcgccttgcttttaacgttgacaattctagctctctcttacgtacgtgaaaaaccctggtcgccggagggaagttccggtgacggaggtaacgaggaggagaaagaggtggagggtggagaagctaaagagtcgacgccagcgcctttttttggggaaatagtcgctgctcttaagaacttgcaaagacctatgaggatcctccttttggtgacgtgtctaaactgggttgcgtggtttcctttcttgttgtttgatactgattggatggggagagaggtgtacggtggtgattcaagtagaaatgctgatcaactgaagatgtatgatcgtggggtccatgcaggtgcattggggttactgcttaactcggtggttttgggattcacttcacttggtgtggaggttttggctcgtggggttggcggggttaaaaggctgtgggggattgtaaattttatcttggctatttgtttggctatgactattttgatcaccaaagtggctcaatctaacagaaggtacaccaccgtgaatggaggaacccacctcttgccgccgccatctggtgtcaaggctggtgctctagccctttttgctgttatgggcatacctcaagctgtaagttgttgttggcttgctttaattctcttcatgtattgttttgtgagtagtaattgaagtcagcctttttggtgcaggtcatctagtttttttttttttttgcatgctcaactgtatcatttttgtcttttactctccttttttgcctttttgttataaaattttattttgaatctggaattttccttttacgtttctttcttcacataaacgtttcctttccctttcccttttttccttttgttttaaattacttgacagttatgaattaaatttttaacataaacttgactgtaaaatcctgttaatcattggtataaattttttacataaacttgatgaagttgttaatcttgctgttgaaatatgtaattgggatttcatagtgtaattagataaggtgcacaacacccccttcggcggcaacagtgtaaatttttcaagcctctagtaacctgtctcctgctcgtgcatggagccacgtagtttaaaactggcaaattttcagagttttggttcctttcagatttcaatggccatccaatgcatgaacaccaaataataaatgcccccgtgggagtgaatgtgtgtttttttggctggtttaggcagcttatgattcttatctgatctttacaaaacttaatgaatgaaaaatcaagaccctttgttttcttctcccgtctccttttgttcttgatcaaccgcaagaaggtcaaacgtacgtgtagttcaacttgcacaagcacagccccctttatcaatgtatctgtgtgcatttaatgagaactggttggttggtagctgctgctgtaattgttgttggaaatgctgcagatgctccttttcgtttaaggattggagatttttcatgatgaactaatggtggcagttattatgcagataacttacagcatcccttttgctttggcatccatcttttctaacacttcgggtgctggccaaggtaagttttttagttccaagcagtacttctgctaaggtatcctcctaaattattaatttccttgtttcaatttgattatagggctttctcttggagttctcaatctttcaatagttataccacaggtcagtatatacacttcatgctaatacatgttgctgctataaattcactctagggattgtttttccatgcccgtttctgtttccaagcattaagcctccacaagtttgtgccttcattttattttgatgatggttgttttaagcaagtccttcagtactactggacaatgccacagtaatgatctgtgaatctggaaaggaaaaaaaaatattcaacaagtattttgcttctttaggatcaaaattgataaagaaatcatcagtccgatgataacctatcttcttctgtcatgattaaacggaatgtttgttagagaagcacacctagacaatatgggctacgatgtcttctgctcttggagttattgaacaggtctacaaacactttttcactagaaagcactaaagggaaagctttggattggattcgaagtatggatgcagtgctggcttctgaaattattgttaaaaaaggatgcataaactttttatagacagaacaaagttctcttgactcatgatcatattctgtgaaccccaagaacgatagactgacattataaccataccattaaaaaattaaaaacaagaagaaaaagaaacgaatctcattcaattcaatgtagtgaagggagagaaaaaagaaagcaattaactctcagttatttaattgattggtgaccgccacatcttgaatttagtttacaaatgattagattagattcatatgtactgcagtatactagaatatgtgcaacagttaattctgaataaatgaatggtaattttaacttgattattgttttaatgatgttatggcagatggtggtttctgtagcagctggaccttgggatgccctttttggaggannnnnnnnnnnnnnnnnnnnnnnnnnnnnnnnnnnnnnnnnnnnnnnnnnacaagagggctcccaccagtagtagtgctgcattccattga

SEQ ID NO: 14 - DNA, 3409 bp, Populus trichocarpa
atggagagtggagttagaaaagaaaacaatcccccttcctcttctttcagtcttcaacaacaacctcctgcaacaaaccctagcccactgagaaagatcataatggttgcatcgatagctgctggtgttcaatttggttgggcgctacagctctctttgttaaccccatatgtgcaactcttgggcattcctcacacatgggctgctttcatctggctttgcggcccgatttctggcatgttagtccagccaacagtggggtactacagtgaccgatgcacctcccgttttggccgtcgtcgcccgtttattgcggcgggggctggttttgttgccatttctgtgtttctcattggctatgccgctgacattggccatctctctggtgattccctcacaaaaacagctaagccacgcgcaattgctgtgtttgtggtagggttctggattcttgatgttgctaataacatgcttcaaggtccatgtagggcatttcttgctgatctttcagggacggatcacaagaagactcgtacagccaatgctttctactctttctttatggctattggcaatgttcttggctttgcatctgggtcttacactcacttgtacagaatctttccattttcaagaaccaaggcttgtgatgtttactgtgcaaatctcaaatcttgtttcttcatctccatcgccttgcttttaacgttgacaattctagctctctcttacgtacgtgaaaaaccctggtcgccggagggaagttccggtgacggaggtaacgaggaggagaaagaggtggagggtggagaagctaaagagtcgacgccagcgcctttttttggggaaatagtcgctgctcttaagaacttgcaaagacctatgaggatcctccttttggtgacgtgtctaaactgggttgcgtggtttcctttcttgttgtttgatactgattggatggggagagaggtgtacggtggtgattcaagtagaaatgctgatcaactgaagatgtatgatcgtggggtccatgcaggtgcattggggttactgcttaactcggtggttttgggattcacttcacttggtgtggaggttttggctcgtggggttggcggggttaaaaggctgtgggggattgtaaattttatcttggctatttgtttggctatgactattttgatcaccaaagtggctcaatctaacagaaggtacaccaccgtgaatggaggaacccacctcttgccgccgccatctggtgtcaaggctggtgctctagccctttttgctgttatgggcatacctcaagctgtaagttgttgtcggcttgctttaattctcttcatgtattgttttgtgagtagtaattgaagtcagcctttttggtgcaggtcatctagtttttttttttttgcatgctcaactgtatcatttttgtcttttactctccttttttgcctttttgttataaaattttattttgaatctggaattttccttttacgtttctttcttcacataaacgtttcctttccctttcccttttttccttttaaattacttgacagttatgaattaaatttttaacataaacttgactgtaaaatcctgttaatcattggtataaattttttacataaacttgatgaagttgttaatcttgatgttgaaatatgtaattgggatttcatagtgtaattagataaggtgcacaacacccccttcggcggcaacagtgtaaatttttcaagcctctagtaacctgtctcctgctcgtgcatggagccacgtatttaaaactggctaattttcagagttttgattcctttcagatttcaatggccatccaatgcatgaacaccaaataataaatgcccccgtgggagtgaatgtgtgtttttttggctggtttaggcagcttatgattcttatctgatctttacaaaacttaatgaatgaaaagtcaagaccctttgttttcttctcccttctccttttgttcttgatcaaccgcaagaaggtcaaacgtacgtgtagttcaacttgcacaagcacagccccctttatcaatgtatctgtgtgcatttaatgagaactggttgattggtagctgctgctgtaattgttgttggaaaagctgcagatgctccttttcgtttaaggattggagatttttcatgatgaactaatggtggcagttattatgcagataacttacagcatcccttttgctttggcatccatcttttctaacacttcgggtgctggccaaggtgagttttttagttccaagcagtacttctgctaaggtatcctcctaaattattaatttccttgtttcaatttgattatagggctttctcttggagttctcaatctttcaatagttataccacaggtcagtatatacacttcatgctaatacatgttgctgctataaattcactctagggattgtttctccatgcccgtttctgtttccaagcattaagcctccacaagtttgtgccttcattttattttgatgatggttgttttaagcaagtccttcagtactactggacaatgccacagtaatgatctgtgaatctggaaaggaaaaaaaaatattcaacaagtattttgcttctttaggatcaaaattgataaagaaatcatcagtccgatgataacctatcttcttctgtcatgattaaacggaatgtttgttagagaagcacacctagacaatatgggctacgatgtcttctgctcttggagttattgaacaggtctacaaacactttttcactagaaagcactaaagggaatgctttggattggattcgaagtatggatgcagtgctggcttctgaaattactgttaaaaaaggatgcataaactttttatagacagaacaaagttctcttgactcatgatcatattctgtgaaccccaagaacgatagactgacattataaccataccattaaaaaattaaaaacaagaagaaaaagaaacgaatctcattcaattcaatgtagtgaagggagagaaaaaagaaagcaattaactctcagttatttaattgattggtgaccgccacatcttgaatttagtttacaaatgattagattagattcatatgtactgcagtatactagaatatgtgcaacagttaattctgaataaatgaatggtaattttaacttgattattgttttaatgatgttatggcagatggtggtttctgtagcagctggaccttgggatgccctttttggaggaggaaatctaccagcttttgttgtaggagcggttgcagctgcagccagtggaatattggcattcaccatgcttccatctccgccccctgacatcccctcaaacaagagggctcccaccagtagtagtgctgcattccattga

SEQ ID NO: 15 - DNA, 24 bp, artificial
  synthetic oligonucleotide primer
tggtktctgtagcrrstggacctt

SEQ ID NO: 16 - DNA, 24 bp, artificial
  synthetic oligonucleotide primer
accagtcaccagtcttggaaggaa

SEQ ID NO: 17 - DNA, 24 bp, artificial
  synthetic oligonucleotide primer
tggtktctgtagcrrstggacctt

SEQ ID NO: 18 - DNA, 24 bp, artificial
  synthetic oligonucleotide primer
ggaatgcakcagtgacagycmttt

SEQ ID NO: 19 - DNA, 24 bp, artificial
  synthetic oligonucleotide primer
atccttgggacttggacaagggtt

SEQ ID NO: 20 - DNA, 24 bp, artificial
  synthetic oligonucleotide primer
tgatcgaggaatacycaagatggc

SEQ ID NO: 21 - DNA, 24 bp, artificial
  synthetic oligonucleotide primer
ataccagcsttygttctggcwtct

SEQ ID NO: 22 - DNA, 25 bp, artificial
  synthetic oligonucleotide primer
ctctgtgaatkcargaycaactagc

SEQ ID NO: 23 - DNA, 24 bp, artificial
  synthetic oligonucleotide primer
ataccagcsttygttctggcwtct

SEQ ID NO: 24 - DNA, 24 bp, artificial
  synthetic oligonucleotide primer
tggaactyggaagaaccatctctc

SEQ ID NO: 25 - DNA, 24 bp, artificial
  synthetic oligonucleotide primer
gaaccttgatcgtcttgagagycg

SEQ ID NO: 26 - DNA, 24 bp, artificial
  synthetic oligonucleotide primer
ggttctgtctccmaacygaaacca

SEQ ID NO: 27 - DNA, 24 bp, artificial
  synthetic oligonucleotide primer
caacctygatcaycgtgagagccg

SEQ ID NO: 28 - DNA, 24 bp, artificial
  synthetic oligonucleotide primer
accattattctggacccggaaccc

SEQ ID NO: 29 - DNA, 24 bp, artificial
  synthetic oligonucleotide primer
tatctgatgctgggctkcaacgga

SEQ ID NO: 30 - DNA, 24 bp, artificial
  synthetic oligonucleotide primer
tgccrgtcmtcgattgacaaaggt

SEQ ID NO: 31 - DNA, 24 bp, artificial
  synthetic oligonucleotide primer
ttatccgacgasggcaatmtatgg

SEQ ID NO: 32 - DNA, 24 bp, artificial
  synthetic oligonucleotide primer
cgaaggcabtgctactgttkttca

SEQ ID NO: 33 - DNA, 22 bp, artificial
  synthetic oligonucleotide primer
aggccacactcaagatttggga

SEQ ID NO: 34 - DNA, 22 bp, artificial
  synthetic oligonucleotide primer
tctcaygtggttgcctcaaggt

SEQ ID NO: 35 - DNA, 24 bp, artificial
  synthetic oligonucleotide primer
gctattcawgaagaagctcgcctg

SEQ ID NO: 36 - DNA, 24 bp, artificial
  synthetic oligonucleotide primer
agacactaaagcagactgcagagg

SEQ ID NO: 37 - DNA, 24 bp, artificial
  synthetic oligonucleotide primer
tgctttggcygagaagagacttma

SEQ ID NO: 38 - DNA, 24 bp, artificial
  synthetic oligonucleotide primer
ggccacthgtgttaagyccacaaa

SEQ ID NO: 39 - DNA, 24 bp, artificial
  synthetic oligonucleotide primer
aacaagcacryctgttccagacat

SEQ ID NO: 40 - DNA, 24 bp, artificial
  synthetic oligonucleotide primer
tctgwccacgmtttctccttggrt

SEQ ID NO: 41 - DNA, 24 bp, artificial
  synthetic oligonucleotide primer
tttatggttgctgactgctgcrtg

SEQ ID NO: 42 - DNA, 24 bp, artificial
  synthetic oligonucleotide primer
ccagcatcatytttgccaccaagt

SEQ ID NO: 43 - DNA, 24 bp, artificial
  synthetic oligonucleotide primer
gcttctcacmgcrgcatgcatmaa

SEQ ID NO: 44 - DNA, 24 bp, artificial
  synthetic oligonucleotide primer
tcytcgagtgccaccrtrcccaaa

SEQ ID NO: 45 - DNA, 26 bp, artificial
  synthetic oligonucleotide primer
ttgaaaggagyctgtagcagtgcaag

SEQ ID NO: 46 - DNA, 24 bp, artificial
  synthetic oligonucleotide primer
tgagacaagcaggcagagactgtt

SEQ ID NO: 47 - DNA, 26 bp, artificial
  synthetic oligonucleotide primer
ttgaaaggagyctgtagcagtgcaag

SEQ ID NO: 48 - DNA, 24 bp, artificial
  synthetic oligonucleotide primer
gagcarccaggcaaagacttgtga

SEQ ID NO: 49 - DNA, 24 bp, artificial
  synthetic oligonucleotide primer
atgtctttgcttacgccgatgacc

SEQ ID NO: 50 - DNA, 24 bp, artificial
  synthetic oligonucleotide primer
tcatatgctgctcttgcgctctca

SEQ ID NO: 51 - DNA, 24 bp, artificial
  synthetic oligonucleotide primer
agagagtcctgacaatgggcttca

SEQ ID NO: 52 - DNA, 24 bp, artificial
  synthetic oligonucleotide primer
gytgggttgccrttctcaakttca

SEQ ID NO: 53 - DNA, 20 bp, artificial
  synthetic oligonucleotide primer
aagaggacaagaaggcagca

SEQ ID NO: 54 - DNA, 20 bp, artificial
  synthetic oligonucleotide primer
ctaaccgccttctccaacac

SEQ ID NO: 55 - DNA, 24 bp, artificial
  synthetic oligonucleotide primer
ctgaagaaggagatgacarcmcca

SEQ ID NO: 56 - DNA, 24 bp, artificial
  synthetic oligonucleotide primer
gcatcccttcaacacagtttcamg

SEQ ID NO: 57 - DNA, 24 bp, artificial
  synthetic oligonucleotide primer
ttatccgacgasggcaatmtatgg

SEQ ID NO: 58 - DNA, 24 bp, artificial
  synthetic oligonucleotide primer
ggtacagatggatgcaaattaggt

SEQ ID NO: 59 - DNA, 24 bp, artificial
  synthetic oligonucleotide primer
agaaytgccatcwcatcyagggtt

SEQ ID NO: 60 - DNA, 22 bp, artificial
  synthetic oligonucleotide primer
ttcattgcgtggattctcyccc

SEQ ID NO: 61 - DNA, 24 bp, artificial
  synthetic oligonucleotide primer
agaaytgccatcwcatcyagggtt

SEQ ID NO: 62 - DNA, 26 bp, artificial
  synthetic oligonucleotide primer
gtacgggacaatttcatcatccattg

SEQ ID NO: 63 - DNA, 24 bp, artificial
  synthetic oligonucleotide primer
tttcgtagacatggatcctcgcca

SEQ ID NO: 64 - DNA, 24 bp, artificial
  synthetic oligonucleotide primer
tggcttccttcgtttcgtggtaga

SEQ ID NO: 65 - DNA, 24 bp, artificial
  synthetic oligonucleotide primer
agaygtggtgacaaagggttctga

SEQ ID NO: 66 - DNA, 24 bp, artificial
  synthetic oligonucleotide primer
tgagacargggaagggttaaggga

SEQ ID NO: 67 - DNA, 24 bp, artificial
  synthetic oligonucleotide primer
tactgaagggagttggcagcagtt

SEQ ID NO: 68 - DNA, 26 bp, artificial
  synthetic oligonucleotide primer
gcctctatctcggcagatcgaagaaa

SEQ ID NO: 69 - DNA, 24 bp, artificial
  synthetic oligonucleotide primer
agaygtggtgacaaagggttctga

SEQ ID NO: 70 - DNA, 24 bp, artificial
  synthetic oligonucleotide primer
tgttgggcagctctgtagaccaaa

SEQ ID NO: 71 - DNA, 24 bp, artificial
  synthetic oligonucleotide primer
aggcttagtggaatacggcagtga

SEQ ID NO: 72 - DNA, 27 bp, artificial
  synthetic oligonucleotide primer
cccatagccactagagctgctgagata

SEQ ID NO: 73 - DNA, 24 bp, artificial
  synthetic oligonucleotide primer
caatcaaggtggcccagcagaaat

SEQ ID NO: 74 - DNA, 24 bp, artificial
  synthetic oligonucleotide primer
gtagatgcgttggagaccagttgc

SEQ ID NO: 75 - DNA, 24 bp, artificial
  synthetic oligonucleotide primer
caatcaaggtggcccagcagaaat

SEQ ID NO: 76 - DNA, 24 bp, artificial
  synthetic oligonucleotide primer
atagatgcgttgaagaccagctgc

SEQ ID NO: 77 - DNA, 24 bp, artificial
  synthetic oligonucleotide primer
tggatcccggacactggaataagt

SEQ ID NO: 78 - DNA, 24 bp, artificial
  synthetic oligonucleotide primer
tctgagrctggtgtttgascttct

SEQ ID NO: 79 - DNA, 24 bp, artificial
  synthetic oligonucleotide primer
atggagagtggagttagaaaagaa

SEQ ID NO: 80 - DNA, 24 bp, artificial
  synthetic oligonucleotide primer
accagtcaccagtcttggaaggaa

SEQ ID NO: 81 - DNA, 24 bp, artificial
  synthetic oligonucleotide primer
atggagagtggagttagaaaagaa

SEQ ID NO: 82 - DNA, 33 bp, artificial
  synthetic oligonucleotide primer
tcaawggaatgcarcaswastactggtggsagc

SEQ ID NO: 83 - DNA, 24 bp, artificial
  synthetic oligonucleotide primer
atggagagtggagttagaaaagaa

SEQ ID NO: 84 - DNA, 25 bp, artificial
  synthetic oligonucleotide primer
tcaatggaatgcakcagtgacagyc

SEQ ID NO: 85 - DNA, 32 bp, artificial
  synthetic oligonucleotide primer
ctagctagcatgtcagtcgctaacccagagcc

SEQ ID NO: 86 - DNA, 36 bp, artificial
  synthetic oligonucleotide primer
ccgctcgagtcatgagaagaccatgggcttttgaac

SEQ ID NO: 87 - DNA, 24 bp, artificial
  synthetic oligonucleotide primer
gtcagtcgctaacccagagccaca

SEQ ID NO: 88 - DNA, 27 bp, artificial
  synthetic oligonucleotide primer
tcatgagaagaccatgggcttttgaac

SEQ ID NO: 89 - DNA, 31 bp, artificial
  synthetic oligonucleotide primer
gctctagatggagtcggcrccgattcgggta

SEQ ID NO: 90 - DNA, 35 bp, artificial
  synthetic oligonucleotide primer
ccgctcgagtagcatgctcctgtccttgacaatca

SEQ ID NO: 91 - DNA, 24 bp, artificial
  synthetic oligonucleotide primer
atggagtcggcrccgattcgggta

SEQ ID NO: 92 - DNA, 30 bp, artificial
  synthetic oligonucleotide primer
ttagccaaaatgaaaaccactgaaggaact

(19) United States
(12) Patent Application Publication
(10) Pub. No.: US 2013/0291229 A1
(43) Pub. Date: Oct. 31, 2013

(54) MODIFICATION OF SUCROSE DISTRIBUTION IN PLANTS

(71) Applicants: Chung-Jui Tsai, Athens, GA (US); Scott A. Harding, Athens, GA (US)

(72) Inventors: Chung-Jui Tsai, Athens, GA (US); Scott A. Harding, Athens, GA (US)

(73) Assignee: UNIVERSITY OF GEORGIA RESEARCH FOUNDATION, INC., Athens, GA (US)

(21) Appl. No.: 13/884,414

(22) PCT Filed: Nov. 18, 2011
(86) PCT No.: PCT/US11/61362

§ 371 Date: Jul. 16, 2013

Related U.S. Application Data
(60) Provisional application No. 61/415,145, filed on Nov. 18, 2010.

(51) Int. Cl. C12N 15/82

(52) U.S. Cl. CPC C12N 15/8245
USPC 800/298; 435/277; 435/165; 162/100

(57) ABSTRACT

The present invention provides transgenic plant and methods of using them. In one embodiment, the methods include growing a transgenic plant under certain conditions, such as stress conditions, such as drought, heat, or salt, where the plant has decreased expression of a SUT polypeptide compared to a control plant. The transgenic plant may have a phenotype of increased growth, increased formation of woody tissue, increased drought tolerance, increased water use efficiency, increased nitrogen utilization efficiency, or a combination thereof, compared to the control plant. In another embodiment, the methods include using a transgenic plant to produce a pulp.

### CROSS-REFERENCE TO RELATED APPLICATIONS

This application claims the benefit of U.S. Provisional Application Ser. No. 61/415,145, filed Nov. 18, 2010, which is incorporated by reference herein.

### GOVERNMENT FUNDING

The present invention was made with government support under Grant No. DBI-0421756 and DBI-0836433, awarded by the NSF, and Grant No. DE-SC0005140, awarded by DOE. The Government has certain rights in this invention.

### BACKGROUND

Sucrose delivered from photosynthetic source organs to heterotrophic sink organs includes the raw carbon for biosynthetic reactions that yield food as well as lignocellulose. Sucrose is loaded into, transported through and unloaded from the phloem in a series of steps underpinned by sucrose transporters (SUTs) (Slewinski and Braun, (2010) Plant Sci., 178, 341-349). These processes differ in important ways between fast-growing, herbaceous annual and slower growing, wood-forming perennial species (Rennie and Turgeon, (2009) Proc. Natl. Acad. Sci. USA, 106, 14162-14167, Van Bel et al., (1992) Plant Cell Environ., 15, 265-270). In Arabidopsis, many of the Solanaceous species and monocots, there is little symplastic continuity between the phloem companion cells and the photosynthetic mesophyll, and phloem loading occurs from the apoplast (Barker et al., (2000) Plant Cell, 12, 1153-1164, Knop et al., (2001) Planta, 213, 80-91, Riesmeier et al., (1993) Plant Cell, 5, 1591-1598, Schmitt et al., (2008) Plant Physiol., 148, 187-199, Stadler and Sauer, (1996) Botanica Acta, 109, 299-306, Van Bel et al., (1992) Plant Cell Environ., 15, 265-270). Proton-coupled Group I (monocot) and Group II (dicot) SUTs in the plasmalemma mediate apoplastic phloem loading, and they are essential for normal growth of these species (Burkle et al., (1998) Plant Physiol., 118, 59-68, Gottwald et al., (2000) Proc. Natl. Acad. Sci. USA, 97, 13979-13984, Hackel et al., (2006) Plant J., 45, 180-192, Riesmeier et al., (1994) EMBO J., 13, 1-7, Srivastava et al., (2008) PlantPhysiol., 148, 200-211). In contrast, temperate tree species can exhibit high degrees of symplastic, plasmodesmatal connectivity between phloem companion cells and the mesophyll (Gamalei, (1989) Trees-Struct. Funct., 3, 96-110, Rennie and Turgeon, (2009) Proc. Natl. Acad. Sci. USA, 106, 14162-14167). Phloem loading in these species is poorly understood, but is thought to occur at least partly via the symplasm, and to be energetically passive and comparatively inefficient for sucrose export (Korner et al., (1995) Plant Cell Environ., 18, 595-600, Rennie and Turgeon, (2009) Proc. Natl. Acad. Sci. USA, 106, 14162-14167, Van Bel et al., (1992) Plant Cell Environ., 15, 265-270). Arguments for strict apoplastic phloem loading have been presented, but currently there is no consensus about whether SUT proteins have a role, or whether symplastic export is a significant contributor to the process in these species (Rennie and Turgeon, (2009) Proc. Natl. Acad. Sci. USA, 106, 14162-14167, Russin and Evert, (1985) Am. J. Bot., 72, 1232-1247, Turgeon, (2010) Plant Physiol., 152, 1817-1823, Turgeon and Medville, (2004) Plant Physiol., 136, 3795-3803).

After phloem loading, differences in sucrose distribution between herbaceous and tree species can lie in the contrasting longevity, structural complexity and positioning of major sink organs relative to the transport path. Whereas the major sink organs in herbaceous annuals are terminal (e.g., seed and tuber), the major sinks in trees include lateral wood-forming and other specialized tissues that exhibit annual cycles of carbohydrate storage and re-mobilization for growth. SUT proteins, mode of phloem loading, and thermodynamic properties of the transport stream all interact during the distribution of photoassimilates to lateral and terminal sinks (Hafke et al., (2005) Plant Physiol., 138, 1527-1537, Srivastava et al., (2008) PlantPhysiol., 148, 200-211, Van Bel, (2003) Plant Physiol., 131, 1509-1510). In the stems of woody species, symplastic as well as apoplastic exchange of various metabolites, including sugars, occurs across the cambium and between xylem and phloem (Fuchs et al., (2010) Ann. Bot., 105, 375-387, Van Bel, (1990) J. Exp. Bot., 41, 631-644, Van Bel, (2003) Plant Physiol., 131, 1509-1510), but SUT participation in that process has not been reported. The stem-specific, Group II walnut (Juglans regia) JrSUT1 is thought to have a role in the apoplastic distribution of sucrose within the xylem between the vessels and vessel-associated parenchyma during seasonal freeze-thaw cycles (Alves et al., (2004) Tree Physiol., 24, 99-105, Decourteix et al., (2006) Plant Cell Environ., 29, 36-47). Such seasonal redistributions of sucrose have also been reported in Populus and Salix species (Sauter, (1983) Zeitschrift Fur Pflanzenphysiologie, 111, 429-440, Sauter, (1988) J Plant Physiol., 132, 608-612). JrSUT1 also appears to have a role in mediating the export of sucrose from xylem to leaves emerging after winter dormancy (Decourteix et al., (2008) Tree Physiol., 28, 215-224).

Multiple SUT isoforms are responsible for phloem loading, transport and unloading of sucrose in herbaceous species as recently reviewed (Braun and Slewinski, (2009) Plant Physiol., 149, 71-81), but the understanding of plant-wide SUT function in species valued for wood production is much less comprehensive. In herbaceous species at least, certain source leaf-expressed plasma membrane SUT proteins exhibit rapid turnover and are under tight transcriptional control as a function of sink demand (Kuhn, (2003) Plant Biol., 5, 215-232, Vau hn et al., (2002) Proc. Natl. Acad. Sci. USA, 99, 10876-10880). Tonoplast SUT proteins (Group IV) which can mediate subcellular compartmentation of sucrose (Endler et al., (2006) Plant Physiol., 141, 196-207, Reinders et al., (2008) Plant Mol. Biol., 68, 289-299) are minor constituents in herbaceous species. Their regulation by sink-source relations has not been investigated, and their occurrence in woody perennials has not been reported. To date, gene silencing has been used in the functional analysis of one non-tonoplast Group IV transporter, the plasma/ER membrane-localized Solanum tuberosum (potato) StSUT4 (Chincinska et al., (2008) Plant Physiol., 146, 515-528). StSUT4-silenced plants displayed altered diurnal rhythms, early flowering, increased tuber yield, and reduced expression of gibberellic acid and ethylene biosynthetic genes (Chincinska et al., (2008) Plant Physiol., 146, 515-528). No genetic characterization based on mutant or transgenic silencing has been reported for tonoplast-localized SUTs.

### SUMMARY OF THE INVENTION

Provided herein are methods for using a transgenic plant. In one embodiment, the methods include growing a transgenic plant under certain conditions, such as drought conditions, where the plant has decreased expression of a SUT polypeptide compared to a control plant. The transgenic plant may have a phenotype of increased growth, increased formation of woody tissue, increased drought tolerance, increased water use efficiency, increased nitrogen utilization efficiency, or a combination thereof, compared to the control plant. The SUT polypeptide may have at least 80% amino acid sequence identity to SEQ ID NO:2. The expression of the SUT polypeptide may be decreased in the transgenic plant by at least 10% when grown under the certain conditions compared to the transgenic plant not grown in the condition. The plant may be a dicot, such as a woody plant, for instance, a member of the genus Populus.

In another embodiment, the methods are for maintaining stem growth during exposure to a stress condition. The stress condition may be selected from drought, heat, salt, or the combination thereof. The method may include growing a transgenic plant under stress conditions, where the transgenic plant has decreased expression of a coding region encoding a SUT polypeptide compared to a control plant. The expression of the SUT polypeptide may be decreased in the transgenic plant by at least 10% when grown in a stress condition compared to the transgenic plant not grown in the stress condition. The plant may be a dicot, such as a woody plant, for instance, a member of the genus Populus.

In another embodiment, the methods are for using a plant having increased water use efficiency. The method may include growing a transgenic plant under conditions of decreased water availability, where the transgenic plant has decreased expression of a coding region encoding a SUT polypeptide compared to a control plant.

In one embodiment, the methods are for using a transgenic plant that has decreased expression of a coding region encoding a SUT polypeptide compared to a control plant. The method may include processing a transgenic plant to result in pulp. The processing may include a physical pretreatment, a chemical pretreatment, or a combination thereof. The method may further include hydrolyzing the pulp. The method may further include contacting the pulp with a microbe under conditions suitable for fermentation of the pulp. The method may further include obtaining a metabolic product, such as ethanol. Also provided herein is a pulp made using a transgenic plant described herein.

Provided herein are transgenic plants, and parts of a transgenic plant, such as a leaf, a stem, a flower, an ovary, a fruit, a seed, and a callus. In one embodiment, a transgenic plant includes an exogenous polynucleotide, wherein expression of the exogenous polynucleotide inhibits expression of a SUT polypeptide. The exogenous polynucleotide may be operably linked to a promoter responsive to a stress condition. The exogenous polynucleotide may include at least 19 nucleotides that are substantially identical or substantially complementary to SEQ ID NO:1, SEQ NO:3, SEQ ID NO:5, SEQ ID NO:7, or SEQ ID NO:9. The transgenic plant may be a dicot, such as a woody plant, for instance, a member of the genus Populus. Also provided herein are progeny of a transgenic plant (including a progeny that is a hybrid plant), a wood obtained from a transgenic plant, and a wood pulp obtained from a transgenic plant.

As used herein, the term “polynucleotide” refers to a polymeric form of nucleotides of any length, either ribonucleotides or deoxynucleotides, and includes both double- and single-stranded RNA and DNA. A polynucleotide can be obtained directly from a natural source, or can be prepared with the aid of recombinant, enzymatic, or chemical techniques. A polynucleotide can be linear or circular in topology. A polynucleotide may be, for example, a portion of a vector, such as an expression or cloning vector, or a fragment. A polynucleotide may include nucleotide sequences having different functions, including, for instance, coding regions, and non-coding regions such as regulatory regions.

As used herein, the terms “coding region,” “coding sequence,” and “open reading frame” are used interchangeably and refer to a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence. A coding region may be operably linked to heterologous regulatory region. As used herein, a “heterologous” regulatory region, such as a promoter, is a regulatory region that is not normally linked to a particular coding region.

As used herein, an “exogenous coding region” and “exogenous polynucleotide” refers to a polynucleotide that is not normally or naturally found in a cell, or a polynucleotide that is present in a cell by human intervention.

Conditions that are “suitable” for an event to occur, such as expression of an exogenous polynucleotide in a cell to produce a polypeptide, or “suitable” conditions are conditions that do not prevent such events from occurring. Thus, these conditions permit, enhance, facilitate, and/or are conducive to the event.

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, trimers, tetramers). Thus, the terms peptide, oligopeptide, enzyme, subunit, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

As used herein, a polypeptide may be “structurally similar” to a reference polypeptide if the amino acid sequence of the polypeptide possesses a specified amount of sequence similarity and/or sequence identity compared to the reference polypeptide. Thus, a polypeptide may be “structurally similar” to a reference polypeptide if, compared to the reference polypeptide, it possesses a sufficient level of amino acid sequence identity, amino acid sequence similarity, or a combination thereof.

As used herein, an “isolated” substance is one that has been removed from its natural environment, produced using recombinant techniques, or chemically or enzymatically synthesized. For instance, a polypeptide or a polynucleotide can be isolated. Preferably, a substance is purified, i.e., is at least 60% free, preferably at least 75% free, and most preferably at least 90% free from other components with which it is naturally associated.

As used herein, the term “transgenic plant” refers to a plant that has been transformed to contain at least one modification to result in altered expression of a coding region. For example, a coding region in a plant may be modified to include a mutation to reduce transcription of the coding region or reduce activity of a polypeptide encoded by the coding region. Alternatively, a plant may be transformed to include a polynucleotide that interferes with expression of a coding region. For example, a plant may be modified to express an antisense RNA or a double stranded RNA that silences or reduces expression of a coding region by decreasing translation of an mRNA encoded by the coding region. In some embodiments more than one coding region may be affected. The term “transgenic plant” includes whole plant, plant parts (stems, roots, leaves, fruit, etc.) or organs, plant cells, seeds, and progeny of same. A transformed plant of the current invention can be a direct transfectant, meaning that the DNA construct was introduced directly into the plant, such as through Agrobacterium, or the plant can be the progeny of a transfected plant. The second or subsequent generation plant can be produced by sexual reproduction, i.e., fertilization. Furthermore, the plant can be a gametophyte (haploid stage) or a sporophyte (diploid stage). A transgenic plant may have a phenotype that is different from a plant that has not been transformed.

As used herein, the term “control plant” refers to a plant that is the same species as a transgenic plant, but has not been transformed with the same polynucleotide used to make the transgenic plant.

As used herein, the term “plant tissue” encompasses any portion of a plant, including plant cells. Plant cells include suspension cultures, callus, embryos, meristematic regions, callus tissue, leaves, roots, shoots, gametophytes, sporophytes, pollen, seeds and microspores. Plant tissues can be grown in liquid or solid culture, or in soil or suitable media in pots, greenhouses or fields. As used herein, “plant tissue” also refers to a clone of a plant, seed, progeny, or propagule, whether generated sexually or asexually, and descendents of any of these, such as cuttings or seeds.

Unless indicated otherwise, as used herein, “altered expression of a coding region” refers to a change in the transcription of a coding region, a change in translation of an mRNA encoded by a coding region, or a change in the activity of a polypeptide encoded by the coding region.

As used herein, “transformation” refers to a process by which a polynucleotide is inserted into the genome of a plant cell. Such an insertion includes stable introduction into the plant cell and transmission to progeny. Transformation also refers to transient insertion of a polynucleotide, wherein the resulting transformant transiently expresses a polypeptide that may be encoded by the polynucleotide.

As used herein, “phenotype” refers to a distinguishing feature or characteristic of a plant which can be altered according to the present invention by modifying expression of at least one coding region in at least one cell of a plant. The modified expression of at least one coding region can confer a change in the phenotype of a transformed plant by modifying any one or more of a number of genetic, molecular, biochemical, physiological, morphological, or agronomic characteristics or properties of the transformed plant cell or plant as a whole. Whether a phenotype of a transgenic plant is altered is determined by comparing the transformed plant with a plant of the same species that has not been transformed with the same polynucleotide (a “control plant”).

As used herein, “mutation” refers to a modification of the natural nucleotide sequence of a coding region or an operably linked regulatory region made by deleting, substituting, or adding a nucleotide(s) in such a way that the polypeptide encoded by the modified nucleic acid is altered structurally and/or functionally, or the coding region is expressed at an altered level.

As used herein, a “target coding region” and “target coding sequence” refer to a specific coding region whose expression is inhibited by a polynucleotide of the present invention. As used herein, a “target mRNA” is an mRNA encoded by a target coding region.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

### BRIEF DESCRIPTION OF THE FIGURES

FIG. 1. Neighbor-joining tree of predicted SUT amino acid sequences from representative plant species. Sequences from ten sequenced plant genomes obtained from the Phytozome (excluding partial gene models) are shaded. Dicot sequences are indicated with circles, monocot sequences with triangles, and other biochemically or genetically characterized SUT sequences with open symbols. Bootstrap values for major nodes are indicated. The yeast SUT was used as the outgroup. Sequence names and/or accession numbers are provided in Table 3.

FIG. 2. Expression of SUT genes in various Populus tissues. ST, shoot tip; YL, young leaf; ML, mature leaf; SS, secondary stem; Ph, phloem; Xy, xylem; R, root; CC, cell culture; MF, male flower; FF, female flower. Error bars represent the SD of three biological replicates.

FIG. 3. In situ hybridization of SUT transcripts in leaf lamina and transverse sections of primary and secondary stem internodes. (a-e) Source leaf lamina. Minor phloem traces are marked by arrows, and upper palisade cells by arrowheads. Antisense probes were used as indicated, except in (e) where a sense PtaSUT4 probe was used as a negative control. (f-k) Secondary (f-h) and primary (i-k) stem internodes. pf, phloem fiber; ph, phloem; cz, cambial zone; xy, xylem; co, cortex. Scale bar=100 μm.

FIG. 4. Functional characterization of SUT proteins and subcellular localization of PtaSUT4. (a) Complementation of the yeast SUSY7/ura3 mutant by PtaSUT3, PtaSUT4 and PtaSUT5. Transformants were cultured on yeast media supplemented with either 2% glucose (left) or 2% sucrose (right) as sole carbon source. (b) Vacuolar targeting of GFP:PtaSUT4 in Nicotiana benthamiana mesophyllprotoplasts after agroinfiltration. Green channel shows GFP:PtaSUT4 and red channel shows chlorophyll autofluorescence. Chloroplast autofluorescene was localized outside of the GFP-fluorescing tonoplast.

FIG. 5. Altered biomass partitioning in leaf expansion zone of PtaSUT4-RNAi plants. Values represent means and SD of eight plants. P values less than 0.05 are indicated. (a) Ratio of leaf area to stem volume proxies was determined (see Experimental procedures) from LPI-0 through LPI-6 for N-replete plants, and from LPI-0 through LPI-7 for N-limited plants. Older leaves that did not differ in size between genotypes (see FIG. 11) were excluded from the analysis. (b) Total leaf area in expansion zone. (c) Stem height.

FIG. 6. Response of CAZyme and PAL gene expression to PtaSUT4-silencing and plant N-status. QPCR data from three biological replicates were converted to expression ratio and visualized in heatmaps (see Experimental procedures). Yellow indicates no change in expression, while red and blue indicate up- and down-regulation, respectively. Statistical significance of transcript levels between the two samples being compared was determined by Student t-test. White, italicized and underlined fonts indicate a significant difference at p<0.05, and black, italicized and underlined fonts indicate significance atp<0.10. ST, shoot tip; YL, young leaf; ML, mature leaf; SS, secondary stem. (a-b) PtaSUT4-RNAi:WT transcript levels in N-replete (a) or N-limited (b) plants. (c-d) N-limited:N-replete transcript levels in WT (c) or PtaSUT4-RNAi (d) plants.

FIG. 7. Carbohydrate homeostasis in response to PtaSUT4-silencing and plant N-status. Means and SD were obtained from four (shoot tip) or eight biological replicates. Sucrose and starch levels were expressed as glucose equivalent. Statistical significance was determined by Student two-sample t-test. *, p<0.05; **, p<0.01; ***, p<0.001. FIG. 8. Phenolic glycoside and condensed tannin (CT) levels in WT and RNAi plants in response to N-status. Means and SD were obtained from four (shoot tip) or eight replicates. Statistical significance (p<0.05) was determined by Student two-sample t-test.

FIG. 8. Phenolic glycoside and condensed tannin (CT) levels in WT and RNAi plants in response to N-status. Means and SD were obtained from four (shoot tip) or eight replicates. Statistical significance (p<0.05) was determined by Student two-sample t-test.

FIG. 9. Genomic sequence alignment of the two allelic copies (SUT1 and SUT2) with the reference sequences from the Joint Genome Institute Popalr Genome v1.1. SUT1 (JGI), SEQ ID NO:11; SUT1, SEQ ID NO:12; SUT2 (JOT), SEQ ID NO:13; SUT2, SEQ ID NO:14.

FIG. 10. Transgenic lines with RNAi-silenced PtaSUT4 expression. For each plant line transcript levels are in the following order: shoot tip, young leaf, mature leaf, secondary stem, and root.

FIG. 11. Leaf area proxies (lamina length x lamina width) and shoot biomass of transgenic and WT plants.

FIG. 12. Relative transcript abundance of genes depicted in FIG. 6 in various Populus tissues. For each gene transcript levels are in the following order: shoot tip, young leaf, mature leaf, secondary stem.

FIG. 13. Source leaf sugar levels at the beginning and end of the daily photoperiod.

FIG. 14. Multiple sequence alignment of SUT polypeptides. AtSUT4, SEQ ID NO:4; VvSUT11, SEQ ID NO:6; SiSUT4/LeSUT4, SEQ ID NO:8; StSUT4, SEQ ID NO:10; and PtSUT4, SEQ ID NO:2. “:” refers to conserved amino acids, and “*” refers to identical amino acids.

FIG. 15. Plants were grown in 10-gal soil pots supplemented withosmocote slow-release fertilizer. The decrease in soil water content after watering was monitored using tensiometers. At least six plants of each genotype were compared in each experiment.

FIG. 16. Water use efficiency is more sensitive to N source in SUT4-silenced plants. Water uptake was calculated by measuring the decrease in soil water content during a 48 hr or 72 hr period. Water uptake values were normalized by plant height which correlated with shoot biomass. Plants were grown in pots filled with a mixture of 80% vermiculite and 20% perlite. Pots were placed in ebb-and-flow hydroponics tubs and were flooded with the desired nutrient solutions every 72 hrs for 30 minutes. Data represent the mean and SE of 3 plants in each experiment.

FIG. 17. Nucleotide and amino acid sequences of selected coding regions.

FIG. 17-01. Nucleotide sequence and amino acid sequence of PtaSUT4.

FIG. 17-02. Nucleotide sequence and amino acid sequence of AtSUT4.

FIG. 17-03. Nucleotide sequence and amino acid sequence of VvSUT11.

FIG. 17-04. Nucleotide sequence and amino acid sequence of SiSUT4/LeSUT4.

FIG. 17-05. Nucleotide sequence and amino acid sequence of StSUT4.

### DETAILED DESCRIPTION OF ILLUSTRATIVE EMBODIMENTS

Sucrose-proton symporters are one of several trans-membrane proteins that regulate the delivery of substrate carbon from photosynthetic tissues to heterotrophic tissues such as those where wood is formed. The symporters themselves occur as several types that differ in their kinetic properties. The function of plasma-membrane symporters has been widely reported. The function of tonoplast membrane transporters has not been well explored. The Poplar tonoplast transporter has characteristics that distinguish its function from that of similar proteins in herbaceous crop species, and make its function in Poplar relatively more important than in the herbaceous annuals. It is the most strongly expressed of the sucrose-proton symporters in Poplar, while its homologs are among the most weakly expressed of the sucrose-proton symporters in the herbaceous crops. It is very strongly expressed in wood-forming tissues of Poplar, but expression of its homologs in stem vasculature of herbaceous crops is not significant and has not been reported on. We have down-regulated its expression in transgenic Poplar and we have observed that water uptake is reduced in the transgenic Poplars, but water use efficiency is increased. Applications include altered tree growth and stress response, including altered water utilization efficiency.

### Polypeptides

The present invention includes, but is not limited to, a transgenic plant having an alteration in expression of a coding region encoding a sucrose symporter (SUT) polypeptide. In one embodiment, a SUT polypeptides useful herein is tonoplast-localized in a control plant. One example of a SUT is referred to herein as SUT4. Other examples of SUT4 polypeptides include, but are not limited to, SEQ ID NO:2 (ADW94617.1, Populus tremula x alba), SEQ ID NO:4 (AAL59915.1, Arabidopsis thaliana), SEQ ID NO:6 (AAF08329.1, Vitis vinifera), SEQ ID NO:8 (AAG09270.1, Solanum lycopersicum [Lycopersion esculentum]), and SEQ ID NO:10 (AAG25923.2, Solanum tuberosum).

Other examples of SUT polypeptides include those that are structurally similar the amino acid sequence of SEQ ID NO:2, SEQ ID NO:4, SEQ ID NO:6, SEQ ID NO:8, or SEQ ID NO:10. A SUT polypeptide that is structurally similar to the amino acid sequence of a polypeptide described herein has biological activity. Methods for testing whether a polypeptide has biological activity are described below.

Structural similarity of two polypeptides can be determined by aligning the residues of the two polypeptides (for example, a candidate polypeptide and any appropriate reference polypeptide described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A reference polypeptide may be a polypeptide described herein. A candidate polypeptide is the polypeptide being compared to the reference polypeptide. A candidate polypeptide may be isolated, for example, from a plant, or can be produced using recombinant techniques, or chemically or enzymatically synthesized. A candidate polypeptide may be inferred from a nucleotide sequence present in the genome of a plant.

Unless modified as otherwise described herein, a pair-wise comparison analysis of amino acid sequences can be carried out using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on. Alternatively, polypeptides may be compared using the BESTFIT algorithm in the GCG package (version 10.2, Madison Wis.).

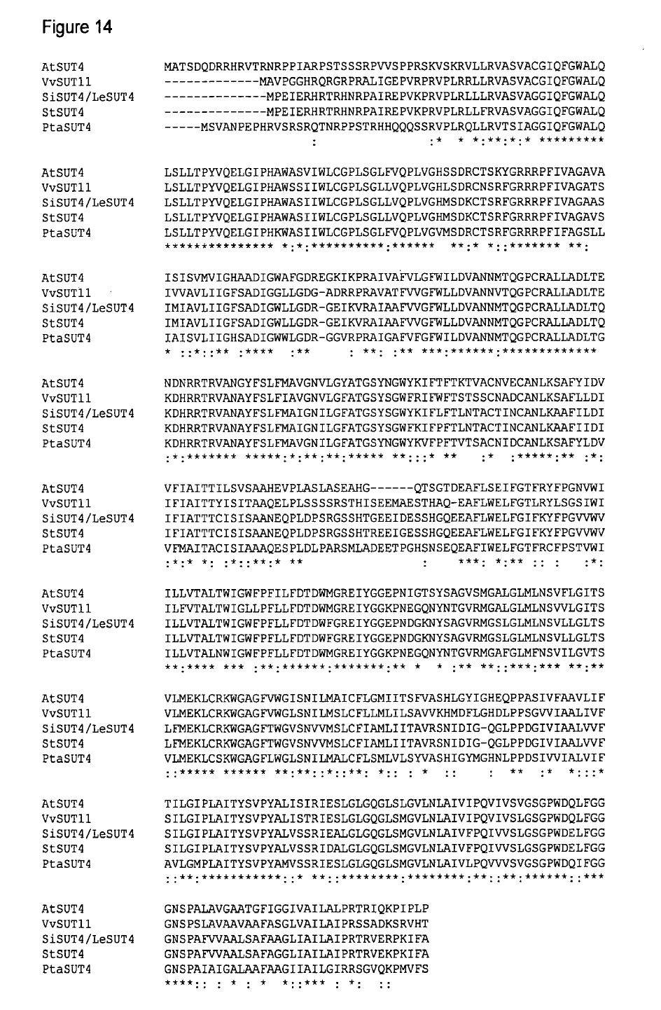

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide described herein may be selected from other members of the class to which the amino acid belongs. For example, it is known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2. As can be seen in FIG. 14, SEQ ID NOs:2, 4, 6, 8, and 10 are shown in a multiple protein alignment. Identical and conserved amino acids are marked with a “*” and a “:” respectively.

Thus, as used herein, a candidate polypeptide useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence.

Alternatively, as used herein, a candidate polypeptide useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

The SUT polypeptides contain conserved domains, including but not limited to (1) the GPH_sucrose domain (TIGRO1301) found in the plant glycoside-pentoside-hexuronide (GPH)/cation symporter family, and (2) the MFS (major facilitator superfamily) domain found in a diverse group of secondary transporters that include uniporters, symporters and antiporters. The SUT polypeptides also have 12 predicted transmembrane domains.

A SUT polypeptide has biological activity. As used herein, biological activity refers to sucrose transporter activity. In one embodiment, whether a polypeptide has sucrose transporter activity can be determined by measuring the ability of a polypeptide to transport sucrose and hydrogen ions across a plasma membrane. Methods for measuring such activity include the patch-clamp technique and the yeast complementation assay. In one embodiment, whether a polypeptide has sucrose transporter activity can be determined by producing a transgenic plant that has altered expression of a candidate polypeptide and observing the phenotype of the transgenic plant. A transgenic plant altered in the expression of one or more SUT polypeptides may display one or more useful phenotypes as described herein. In one embodiment, expression of a SUT polypeptide in a transgenic plant is decreased. Decreased expression of a SUT polypeptide in a transgenic plant may result in, for instance, reduced water uptake but normal growth, increased water use efficiency, or a combination thereof.

### Polynucleotides

Examples of polynucleotides encoding SEQ ID NO:2, SEQ ID NO:4, SEQ ID NO:6, SEQ ID NO:8, and SEQ ID NO: 10 are shown at SEQ ID NO:1, SEQ ID NO:3, SEQ ID NO: 5, SEQ ID NO:7, and SEQ ID NO: 9, respectively. It should be understood that a polynucleotide encoding one of the SUT polypeptides is not limited to a nucleotide sequence disclosed herein, but also includes the class of polynucleotides encoding the SUT polypeptide as a result of the degeneracy of the genetic code. For example, the naturally occurring nucleotide sequence SEQ Ill NO:1 is but one member of the class of nucleotide sequences encoding a polypeptide having the amino acid sequence SEQ ID NO:2. The class of nucleotide sequences encoding a selected polypeptide sequence is large but finite, and the nucleotide sequence of each member of the class may be readily determined by one skilled in the art by reference to the standard genetic code, wherein different nucleotide triplets (codons) are known to encode the same amino acid.

While the polynucleotide sequences described herein are listed as DNA sequences, it is understood that the complements, reverse sequences, and reverse complements of the DNA sequences can be easily determined by the skilled person. It is also understood that the sequences disclosed herein as DNA sequences can be converted from a DNA sequence to an RNA sequence by replacing each thymidine nucleotide with a uracil nucleotide.

Sequence similarity of two polynucleotides can be determined by aligning the residues of the two polynucleotides (for example, a candidate polynucleotide and any appropriate reference polynucleotide described herein) to optimize the number of identical nucleotides along the lengths of their sequences; gaps in either or both sequences are permitted in making the alignment in order to optimize the number of identical nucleotides, although the nucleotides in each sequence must nonetheless remain in their proper order. A reference polynucleotide may be a polynucleotide described herein. A candidate polynucleotide is the polynucleotide being compared to the reference polynucleotide. A candidate polynucleotide may be isolated, for example, from a plant, or can be produced using recombinant techniques, or chemically or enzymatically synthesized. A candidate polynucleotide may be present in the genome of a plant and predicted to encode a SUT polypeptide.

Unless modified as otherwise described herein, a pair-wise comparison analysis of nucleotide sequences can be carried out using the Blastn program of the BLAST search algorithm, available through the World Wide Web, for instance at the internet site maintained by the National Center for Biotechnology Information, National Institutes of Health. Preferably, the default values for all Blastn search parameters are used. Alternatively, sequence similarity may be determined, for example, using sequence techniques such as GCG FastA (Genetics Computer Group, Madison, Wis.), MacVector 4.5 (Kodak/IBI software package) or other suitable sequencing programs or methods known in the art.

Thus, as used herein, a candidate polynucleotide useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% nucleotide sequence identity to a reference nucleotide sequence.

The present invention also provides methods of using SUT polypeptides and polynucleotides encoding SUT polypeptides. The present invention includes methods for altering expression of plant SUT coding regions for purposes including, but not limited to (i) investigating SUT polypeptide expression and its effect on plant phenotype, (ii) effecting a change in plant phenotype, and (iii) using plants having an altered phenotype.

The present invention includes methods for altering, for instance, decreasing, the expression of a coding region encoding a SUT polypeptide. Thus, for example, the invention includes altering expression of a SUT coding region present in the genome of a wild-type plant. As disclosed herein, in one embodiment a wild-type plant is a woody plant, such as a member of the species Populus.

Techniques which can be used in accordance with the present invention to alter expression of a SUT coding region, include, but are not limited to: (i) modifying the timing of expression of the coding region by placing it under the control of a non-native promoter, (ii) disrupting a coding region's transcript, such as disrupting a coding region's mRNA transcript; (iii) disrupting the function of a polypeptide encoded by a coding region, or (iv) disrupting the coding region itself. Techniques for altering expression of coding regions in a plant are valuable for discovering the functional effects of a coding region and for generating plants with a phenotype that is different from a wild-type plant of the same species.

Antisense RNA, ribozyme, and dsRNAi technologies typically target RNA transcripts of coding regions, usually mRNA. Antisense RNA technology involves expressing in, or introducing into, a cell an RNA molecule (or RNA derivative) that is complementary to, or antisense to, sequences found in a particular mRNA in a cell. By associating with the mRNA, the antisense RNA can inhibit translation of the encoded gene product. The use of antisense technology to reduce or inhibit the expression of specific plant genes has been described, for example in European Patent Publication No. 271988, Smith et al., 1988, Nature, 334:724-726; Smith et. al., 1990, Plant Mol. Biol., 14:369-379.

A ribozyme is an RNA that has both a catalytic domain and a sequence that is complementary to a particular mRNA. The ribozyme functions by associating with the mRNA (through the complementary domain of the ribozyme) and then cleaving (degrading) the message using the catalytic domain.

RNA interference (RNAi) involves a post-transcriptional gene silencing (PTGS) regulatory process, in which the steady-state level of a specific mRNA is reduced by sequence-specific degradation of the transcribed, usually fully processed mRNA without an alteration in the rate of de novo transcription of the target gene itself. The RNAi technique is discussed, for example, in Small, 2007, Curr. Opin. Biotechnol., 18:148-153; McGinnis, 1010, Brief. Funct. Genomics, 9(2): 111-117.

Disruption of a coding region may be accomplished by T-DNA based inactivation. For instance, a T-DNA may be positioned within a polynucleotide coding region described herein, thereby disrupting expression of the encoded transcript and protein. T-DNA based inactivation can be used to introduce into a plant cell a mutation that alters expression of the coding region, e.g., decreases expression of a coding region or decreases activity of the polypeptide encoded by the coding region. For instance, mutations in a coding region and/or an operably linked regulatory region may be made by deleting, substituting, or adding a nucleotide(s). The use of T-DNA based inactivation is discussed, for example, in Azpiroz-Leehan et al. (1997, Trends in Genetics, 13:152-156).

Altering expression of a SUT coding region may be accomplished by using a portion of a polynucleotide described herein. In one embodiment, a polynucleotide for altering expression of a SUT coding region in a plant cell includes one strand, referred to herein as the sense strand, of at least 19 nucleotides, for instance, 19, 20, 21, 22, 23, 24, 25, 26, 27, 28, or 29 nucleotides (e.g., lengths useful for dsRNAi and/or antisense RNA). In one embodiment, a polynucleotide for altering expression of a SUT coding region in a plant cell includes substantially all of a coding region, or in some cases, an entire coding region (e.g., lengths useful for T-DNA based inactivation). The sense strand is substantially identical, preferably, identical, to a target coding region or a target mRNA. As used herein, the term “identical” means the nucleotide sequence of the sense strand has the same nucleotide sequence as a portion of the target coding region or the target mRNA. As used herein, the term “substantially identical” means the sequence of the sense strand differs from the sequence of a target mRNA at least 1%, 2%, 3%, 4%, or 5% of the nucleotides, and the remaining nucleotides are identical to the sequence of the mRNA.

In one embodiment, a polynucleotide for altering expression of a SUT coding region in a plant cell includes one strand, referred to herein as the antisense strand. The antisense strand may be at least 19 nucleotides, for instance, 19, 20, 21, 22, 23, 24, 25, 26, 27, 28, or 29 nucleotides. In one embodiment, a polynucleotide for altering expression of a SUT coding region in a plant cell includes substantially all of a coding region, or in some cases, an entire coding region. An antisense strand is substantially complementary, preferably, complementary, to a target coding region or a target mRNA. As used herein, the term “substantially complementary” means that at least 1%, 2%, 3%, 4%, or 5% of the nucleotides of the antisense strand are not complementary to a nucleotide sequence of a target coding region or a target mRNA.

Methods are readily available to aid in the choice of a series of nucleotides from a polynucleotide described herein. For instance, algorithms are available that permit selection of nucleotides that will function as dsRNAi and antisense RNA for use in altering expression of a coding region. The selection of nucleotides that can be used to selectively target a coding region for T-DNA based inactivation may be aided by knowledge of the nucleotide sequence of the target coding region.

Polynucleotides described herein, including nucleotide sequences which are a portion of a coding region described herein, may be operably linked to a regulatory sequence. An example of a regulatory region is a promoter. A promoter is a nucleic acid, such as DNA, that binds RNA polymerase and/or other transcription regulatory elements. A promoter facilitates or controls the transcription of DNA or RNA to generate an RNA molecule from a nucleic acid molecule that is operably linked to the promoter. The RNA can encode an antisense RNA molecule or a molecule useful in RNAi. Promoters may be used for expression of a polynucleotide in a particular tissue or intracellular environment, examples of which are known to one of ordinary skill in the art. Promoters useful in the invention include constitutive promoters, inducible promoters, tissue preferred promoters, and/or promoters responsive to certain conditions. In one embodiment, a polynucleotide useful for decreasing expression of a SUT polypeptide may be operably linked to a promoter that is responsive to drought conditions. SUT polypeptide expression in such a transgenic plant would be decreased to a greater degree when the plant was exposed to drought conditions.

Examples of useful constitutive plant promoters include, but are not limited to, the cauliflower mosaic virus (CaMV) 35S promoter, (Odel et al., 1985, Nature, 313:810), the nopaline synthase promoter (An et al., 1988, Plant Physiol., 88:547), and the octopine synthase promoter (Fromm et al., 1989, Plant Cell 1:977).

Examples of inducible promoters include, but are not limited to, auxin-inducible promoters (Baumann et al., 1999, Plant Cell, 11:323-334), cytokinin-inducible promoters (Guevara-Garcia, 1998, Plant Mol. Biol., 38:743-753), and gibberellin-responsive promoters (Shi et al., 1998, Plant Mol. Biol., 38:1053-1060). Additionally, promoters responsive to heat (Schöffl et al., 1989, Mol. Gen. Genet., 217:246-253), drought (Medrano et al., U.S. Pat. No. 7,314,757), stress (such as drought, cold, and salt, etc., Yamaguchi-Shinozaki et al., 1994, Plant Cell, 6:251-264) light, wounding, pathogen resistance, and chemicals such as methyl jasmonate or salicylic acid, can be used, as can tissue or cell-type specific promoters such as xylem-specific promoters (Lu et al., 2003, Plant Growth Regulation 41:279-286). Examples of tissue-specific promoters include, but are not limited to, xylem-specific promoters (Hu et al., 1998, PNAS 95:5407-5412), phloem-specific promoters (Truernit and Sauer, 1995, Planta 196:564-570), leaf-specific promoters (Kyozuka et al., 1993, Plant Physiol., 102:991-1000) and root-specific promoters (Yamamoto et al., 1991, Plant Cell, 3:371-382).

Another example of a regulatory region is a transcription terminator. Suitable transcription terminators are known in the art and include, for instance, a stretch of 5 consecutive thymidine nucleotides.

Thus, in one embodiment a polynucleotide that is operably linked to a regulatory sequence may be in an “antisense” orientation, the transcription of which produces a polynucleotide which can form secondary structures that affect expression of a target coding region in a plant cell. In another embodiment, the polynucleotide that is operably linked to a regulatory sequence may yield one or both strands of a double-stranded RNA product that initiates RNA interference of a target coding region in a plant cell.

A polynucleotide may be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual., Cold Spring Harbor Laboratory Press (1989). A vector can provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, transposon vectors, and artificial chromosome vectors. A vector may result in integration into a cell's genomic DNA. A vector may be capable of replication in a bacterial host, for instance E. coli. In one embodiment the vector is a plasmid. In some embodiments, a polynucleotide can be present in a vector as two separate complementary polynucleotides, each of which can be expressed to yield a sense and an antisense strand of a dsRNA, or as a single polynucleotide containing a sense strand, an intervening spacer region, and an antisense strand, which can be expressed to yield an RNA polynucleotide having a sense and an antisense strand of the dsRNA.

Selection of a vector depends upon a variety of desired characteristics in the resulting construct, such as a selection marker, vector replication rate, and the like. Suitable host cells for cloning or expressing the vectors herein are prokaryotic or eukaryotic cells. Suitable eukaryotic cells include plant cells. Suitable prokaryotic cells include eubacteria, such as gram-negative organisms, for example, E. coli.

A selection marker is useful in identifying and selecting transformed plant cells or plants. Examples of such markers include, but are not limited to, a neomycin phosphotransferase (nptII) gene (Potrykus et al., 1985, Mol. Gen. Genet., 199:183-188), which confers kanamycin resistance. Cells expressing the nptII gene can be selected using an appropriate antibiotic such as kanamycin or G418. Other commonly used selectable markers include a mutant EPSP synthase gene (Hinchee et al., 1988, Bio/Technology 6:915-922), which confers glyphosate resistance; and a mutant acetolactate synthase gene (ALS), which confers imidazolinone or sulphonylurea resistance (Conner and Santino, 1985, European Patent Application 154,204).

Polynucleotides described herein can be produced in vitro or in vivo. For instance, methods for in vitro synthesis include, but are not limited to, chemical synthesis with a conventional DNA/RNA synthesizer. Commercial suppliers of synthetic polynucleotides and reagents for in vitro synthesis are well known. Methods for in vitro synthesis also include, for instance, in vitro transcription using a circular or linear expression vector in a cell free system. Expression vectors can also be used to produce a polynucleotide of the present invention in a cell, and the polynucleotide may then be isolated from the cell.

### Host Cells, Plants, and Transgenic Plants

The invention also provides host cells having altered expression of a coding region described herein. As used herein, a host cell includes the cell into which a polynucleotide described herein was introduced, and its progeny, which may or may not include the polynucleotide. Accordingly, a host cell can be an individual cell, a cell culture, or cells that are part of an organism. The host cell can also be a portion of an embryo, endosperm, sperm or egg cell, or a fertilized egg. In one embodiment, the host cell is a plant cell.

The present invention further provides transgenic plants having altered expression of a coding region. A transgenic plant may be homozygous or heterozygous for a modification that results in altered expression of a coding region. The level of expression of SUT polypeptide in a transgenic plant may be decreased by at least 10%, at least 20%, at least 30%, at least 40%, at least 50%, at least 60%, at least 70%, at least 80%, at least 90%, or 100%. In one embodiment, the decreased expression may be in a part of the plant, such as, but not limited to, xylem/wood, phloem/bark, leaves, or roots. In one embodiment, the level of expression of SUT polypeptide in a transgenic plant may be decreased when the plant is exposed to certain conditions when compared to the transgenic plant that is not exposed to the condition. Examples of conditions include stress conditions, which includes drought, heat, cold, and salt.

The present invention also includes natural variants of plants, where the natural variants have increased or decreased expression of a SUT polypeptide. In one embodiment, SUT expression is decreased. The change in SUT expression is relative to the level of expression of the SUT polypeptide in a natural population of the same species of plant. Natural populations include natural variants, and at a low level, extreme variants (Studer et al., 2011, 108:6300-6305). The level of expression of SUT polypeptide in an extreme variant may vary from the average level of expression of the SUT polypeptide in a natural population by at least 5%, at least 10%, at least 15%, at least 20%, or at least 25%. The average level of expression of the SUT polypeptide in a natural population may be determined by using at least 50 randomly chosen plants of the same species as the putative extreme variant.

The plants may be angiosperms or gymnosperms. The polynucleotides described herein may be used to transform a variety of plants, both monocotyledonous (e.g grasses, corn, grains, oat, wheat, barley), dicotyledonous (e.g., Arabidopsis, tobacco, legumes, alfalfa, oaks, eucalyptus, maple, poplar, aspen, cottonwood), and Gymnosperms (e.g., Scots pine, white spruce, and larch).

The plants also include switchgrass, turfgrass, wheat, maize, rice, sugar beet, potato, tomato, lettuce, carrot, strawberry, cassava, sweet potato, geranium, soybean, and various types of woody plants. Woody plants include trees such as palm oak, pine, maple, fir, apple, fig, plum acacia, sweetgum, poplar, aspen, cottonwood, and willow. Woody plants also include rose and grape vines.

In one embodiment, the plants are woody plants, which are trees or shrubs whose stems live for a number of years and increase in diameter each year by the addition of woody tissue. The invention plants of significance in the commercial biomass industry such as members of the family Salicaceae, such as Populus spp. (e.g., P. trichocarpa, P. deltoides, P. tremula x alba) and members of the genus Salix, as well as pine, and Eucalyptus spp. Also included in the present invention is the wood and wood pulp derived from the plants described herein.

Transformation of a plant with a polynucleotide described herein may yield a phenotype including, but not limited to decreased water uptake, increased thought tolerance, increased water use efficiency, and increased nitrogen utilization efficiency. Phenotype can be assessed by any suitable means. The plants may be evaluated based on their general morphology. Transgenic plants can be observed with the naked eye, can be weighed and their height measured, or subjected to chemical analysis. The plants can be monitored for various photosynthetic parameters, using for example a Licor photosynthesis system, or for their water uptake from soil using tensiometers.

### Methods for Making

Transgenic plants described herein may be produced using routine methods. Methods for transformation and regeneration are known to the skilled person. Transformation of a plant cell with a polynucleotide described herein may be achieved by any known method for the insertion of nucleic acid sequences into a prokaryotic or eukaryotic host cell, including Agrobacterium-mediated transformation protocols, viral infection, whiskers, electroporation, microinjection, polyethylene glycol-treatment, heat shock, lipofection, particle bombardment, and chloroplast transformation.

Transformation techniques for dicotyledons are known in the art and include Agrobacterium-based techniques and techniques that do not require Agrobacterium. Non-Agrobacterium techniques involve the uptake of exogenous genetic material directly by protoplasts or cells. This may be accomplished by PEG or electroporation mediated-uptake, particle bombardment-mediated delivery, or microinjection. In each case the transformed cells may be regenerated to whole plants using standard techniques known in the art.

Techniques for the transformation of monocotyledon species include direct gene transfer into protoplasts using PEG or electroporation techniques, particle bombardment into callus tissue or organized structures, as well as Agrobacterium-mediated transformation.

The cells that have been transformed may be grown into plants in accordance with conventional techniques. See, for example, McCormick et al. (1986, Plant Cell Reports, 5:81-84). These plants may then be grown and evaluated for expression of desired phenotypic characteristics. These plants may be either pollinated with the same transformed strain or different strains, and the resulting hybrid having desired phenotypic characteristics identified. Two or more generations may be grown to ensure that the desired phenotypic characteristics are stably maintained and inherited and then seeds harvested to ensure stability of the desired phenotypic characteristics have been achieved.

### Methods of Use

The present invention also provides methods for using the plants described herein. In various embodiments, the methods include increasing yield, increasing growth, increasing formation of woody tissue, increasing water use efficiency, increasing drought tolerance, and increasing nitrogen utilization efficiency of plants under certain conditions. Plant species vary in their capacity to tolerate drought conditions. For each species, optimal growth can be achieved if a certain level of water is always available. Other factors such as temperature and soil conditions have a significant impact on the availability of water to the plant. “Drought” can be defined as the set of environmental conditions under which a plant will begin to suffer the effects of water deprivation, such as decreased photosynthesis, loss of turgor (wilting) and decreased stomatal conductance. This drought condition results in a significant reduction in yield. Water deprivation may be caused by lack of rainfall or limited irrigation. Alternatively, water deficit may also be caused by high temperatures, low humidity, saline soils, freezing temperatures or water-logged soils that damage roots and limit water uptake to the shoot. Since plant species vary in their capacity to tolerate water deficit, the precise environmental conditions that cause drought stress cannot be generalized. However, drought tolerant plants produce higher biomass and yield than plants that are not drought tolerant. Differences in physical appearance, recovery and yield can be quantified and statistically analyzed using well known measurement and analysis methods. The use of a transgenic plant described herein may result in greater yield during growth in drought conditions compared to a control plant.

“Water use efficiency” is a term that includes various responses to environmental conditions that affect the amount of water available to the plant. For example, under high heat conditions water is rapidly evaporated from both the soil and from the plant itself, resulting in a decrease of available water for maintaining or initiating physiological processes. Likewise, water availability is limited during cold or drought conditions or when there is low water content in the soil. Flood conditions also affect the amount of water available to the plant because it damages the roots and thus limits the plant's ability to transport water to the shoot. As used herein, modulating water use efficiency is intended to encompass all of these situations as well as other environmental situations that affect the plant's ability to use and/or maintain water effectively (e.g. osmotic stress, salinity, etc.). The use of a transgenic plant described herein may result in greater yield during growth in environmental situations that affect the plant's ability to use and/or maintain water effectively compared to a control plant.

In one embodiment, the methods include growing a transgenic plant described herein under stress conditions, such as, but not limited to, decreased water availability, drought, heat, cold, and/or salt. The transgenic plant may have a phenotype of increased growth, increased formation of woody tissue, increased drought tolerance, increased water use efficiency, increased nitrogen utilization efficiency, or a combination thereof, compared to a control plant. In one embodiment, the expression of the SUT polypeptide in the transgenic plant is decreased by at least 10%, at least 20%, at least 30%, at least 40%, at least 50%, at least 60%, at least 70%, at least 80%, at least 90%, or 100% when grown in a stress condition compared to the transgenic plant not grown in the stress condition.

In another embodiment, methods for using the transgenic plants described herein include producing a metabolic product. Examples of metabolic products include, but are not limited to, alcohols, such as ethanol, butanol, a diol, and organic acids such as lactic acid, acetic acid, formic acid, citric acid, oxalic acid, and uric acid.

A process for producing a metabolic product from a transgenic plant described herein may include processing a plant (also referred to as pretreatment of a plant), enzymatic hydrolysis, fermentation, and/or recovery of the metabolic product. Each of these steps may be practiced separately, thus the invention includes methods for processing a transgenic plant to result in a pulp, methods for hydrolyzing a pulp that contain cells from a transgenic plant, and methods for producing a metabolic product from a pulp. Methods for processing a plant, enzymatic hydrolysis, fermentation, and/or recovery of a metabolic product are known to the skilled person and are routine.

Examples of metabolic products include, but are not limited to, alcohols, such as ethanol, butanol, a diol, and organic acids such as lactic acid, acetic acid, formic acid, citric acid, oxalic acid, and uric acid. The method depends upon the metabolic product that is to be recovered, and methods for recovering metabolic products resulting from microbial fermentation of plant material are known to the skilled person and used routinely. For instance, when the metabolic product is ethanol, the ethanol may be distilled using conventional methods. For example, after fermentation the metabolic product, e.g., ethanol, may be separated from the fermented slurry. The slurry may be distilled to extract the ethanol, or the ethanol may be extracted from the fermented slurry by micro or membrane filtration techniques. Alternatively the fermentation product may be recovered by stripping.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

The present invention is illustrated by the following examples. It is to be understood that the particular examples, materials, amounts, and procedures are to be interpreted broadly in accordance with the scope and spirit of the invention as set forth herein.

### Example 1

Plasma membrane, proton-coupled Group II sucrose symporters (SUT) mediate apoplastic phloem loading and sucrose efflux from source leaves in Arabidopsis and agricultural crop species that have been studied to date. We now report that the most abundantly expressed SUT isoform in Populus tremula x alba, PtaSUT4, is a tonoplast (Group IV) symporter. PtaSUT4 transcripts were readily detected in conducting as well as mesophyll cells in stems and source leaves. In comparison, Group II orthologs PtaSUT1 and PtaSUT3 were very weakly expressed in leaves. Both Group II and Group IV SUT genes were well-expressed in secondary stem xylem of Populus. Transgenic poplars with RNAi-suppressed PtaSUT4 exhibited increased leaf-to-stem biomass ratios, elevated sucrose content in source leaves and stems, and altered phenylpropanoid metabolism. Transcript abundance of several carbohydrate-active enzymes and phenylalanine ammonia-lyases was also altered in transgenic source leaves. Nitrogen-limitation led to a down-regulation of vacuolar invertases in all plants, which resulted in an augmentation of sucrose pooling and hexose depletion in source leaves and secondary xylem of the transgenic plants. These results are consistent with a major role for PtaSUT4 in orchestrating the intracellular partitioning, and consequently, the efflux of sucrose from source leaves and the utilization of sucrose by lateral and terminal sinks. Our findings also support the idea that PtaSUT4 modulates sucrose efflux and utilization in concert with plant N-status. This data is also published in Payyavula et al., 2011, Plant Journal, 65:757-770.

### EXPERIMENTAL PROCEDURES

### Plant Materials

Populus tremula x alba clone 717-1134 was used in this study unless otherwise noted. Micropropagated plants were transferred to soil or maintained hydroponically. Pot fertilization and hydroponic nutrient composition were as reported, with modifications where noted (Harding et al., (2009) J. Exp. Bot., 60, 3443-3452). The first unfurled leaf, 3 cm in length, was considered leaf plastochron index (LPI)-0 (Larson and Isebrands, (1971) Can. J. Forest Res., 1, 1-11). Tissues apical to LPI-0 (shoot tip, ST), LPI-1 (sink-source transitional leaf, YL), LPI-8 (mature source leaf, ML), secondary stem internodes 7-9 (SS), phloem and xylem from stem internodes 10-15, and roots (terminal 6-8 cm of elongating roots excluding root tips) were obtained from greenhouse plants. Cell suspension culture (Payyavula et al., (2009) BMC PlantBiol., 9, 151) and male and female flowers from Populus tremuloides Michx. (Oakley et al., (2007) Plant Physiol., 145, 961-973) were obtained as described. Tissues were snap-frozen in liquid nitrogen and stored at −80° C.

### Cloning and Sequence Analysis

SUT gene models were identified from the sequenced poplar genome v1.1 (Tuskan et al., (2006) Science, 313, 1596-1604), using BlastP provided by the Joint Genome Institute, and published SUT protein sequences. Gene-specific primers (Table 1) were designed for cDNA cloning by RT-PCR as described (Rajinikanth et al., (2007) J. Exp. Bot., 58, 1761-1770). PCR amplification of SUT1/2 from genomic DNA extracted from P. trichocarpa Nisqually-1 was performed using rTaq (Takara Bio) and products cloned into pCR11-TOPO (Invitrogen) according to manufacturers' instructions. Molecular weight and theoretical pI of predicted SUT proteins were estimated using the ExPASy proteomic server (available online at ca.expasy.org/tools/pi_tool.html). Phylogenetic tree was constructed using the neighborjoining method in MEGA 4.1 (Kumar et al., (2008) Brief Bioinform, 9, 299-306), with Poisson correction, pairwise deletion and 1,000 bootstrap iterations.

TABLE 1
Gene-specific primers for RT-PCR and various cloning

| | | | | |
|---|---|---|---|---|
| | | | Phytozome v2.2 | |
| Gene | Primers (forward and reverse) | JGI v1.1 gene model | gene model | Reference |

For real-time RT-POR

| | | | | |
|---|---|---|---|---|
| SUT1 | F: TGGTKTCTGTAGCRRSTGGACCTT | gw1.41.182.1 | POPTR_0013s11950 | 1 |
| | R: ACCAGTCACCAGTCTTGGAAGGAA | | | |
| SUT2 | n/a | gw1.459.6.1 | POPTR_0013s11950 | 1 |
| | n/a | | (same as SUT1) | |
| SUT3 | F: TGGTKTCTGTAGCRRSTGGACCTT | gw1.XIX.2155.1 | POPTR_0019s11560 | 1 |
| | R: GGAATGCAKCAGTGACAGYCMTTT | | | |
| SUT4 * | F: ATCCTTGGGACTTGGACAAGGGTT | estExt_fgenesh4_pm.C_ | POPTR_0002s10710, | |
| | R: TGATCGAGGAATACYCAAGATGGC | LG_II0488 | POPTR_0002s10730, | 1 |
| | | | POPTR_0002s10750 | |
| SUT5 | F: ATACCAGCSTTYGTTCTGGCWTCT | fgenesh4_pg.C_LG_ | POPTR_0008s14760 | 1 |
| | R: CTCTGTGAATKCARGAYCAACTAGC | VIII001323 | | |
| SUT6 | F: ATACCAGCSTTYGTTCTGGCWTCT | fgenesh4_pg.C_LG_ | POPTR_0010s10370 | |
| | R: TGGAACTYGGAAGAACCATCTCTC | X000861 | | |
| SUS1 | F: GAACCTTGATCGTCTTGAGAGYCG | estExt_fgenesh4_pm.C_ | POPTR_0018s07380 | 2 |
| | R: GGTTCTGTCTCCMAACYGAAACCA | LG_XVIII0009 | | |
| SUS2 | F: CAACCTYGATCAYCGTGAGAGCCG | estExt_fgenesh4_pg.C_ | POPTR_0006s13900 | 2 |
| | R: ACCATTATTCTGGACCCGGAACCC | 280066 | | |
| SUS3 | F: TATCTGATGCTGGGCTKCAACGGA | estExt_fgenesh4_pm.C_ | POPTR_0002s20340 | 2 |
| | R: TGCCRGTCMTCGATTGACAAAGGT | LG_II0867 | | |
| VIN2 | F: TTATCCGACGASGGCAATMTATGG | estExt_fgenesh4_pg.C_ | POPTR_0003s11210 | 3 |
| | R: CGAAGGCABTGCTACTGTTKTTCA | LG_III0902 | | |
| VIN3 | F: AGGCCACACTCAAGATTTGGGA | estExt_Genewise1_v1. | POPTR_0015s14790 | 3 |
| | R: TCTCAYGTGGTTGCCTCAAGGT | C_LG_XV2841 | | |
| CIN4 | F: GCTATTCAWGAAGAAGCTCGCCTG | estExt_fgenesh4_pg.C_ | POPTR_0006s24400 | 3 |
| | R: AGACACTAAAGCAGACTGCAGAGG | LG_VI1536 | | |
| NIN2/5 ** | F: TGCTTTGGCYGAGAAGAGACTTMA | eugene3.00130058 | POPTR_0013s00800 | 3 |
| | | (NIN2) | (NIN2) | |
| | R: GGCCACTHGTGTTAAGYCCACAAA | gw1.131.249.1 (NIN5) | missing annotation | 3 |
| | | | (NIN5), upstream of | |
| | | | POPTR_0005s01440 | |
| NIN3/4 | F: AACAAGCACRYCTGTTCCAGACAT | gw1.VIII.2341.1 | POPTR_0008s02460 | 3 |
| | | (NIN3) | (NIN3) | |
| | R: TCTGWCCACGMTTTCTCCTTGGRT | gw1.X.3512.1 (NIN4) | POPTR_0010s24250 | 3 |
| | | | (NIN4) | |
| NIN8/12 | F: TTTATGGTTGCTGACTGCTGCRTG | eugene3.00190739 | POPTR_0019s11140 | 3 |
| | | (NIN8) | (NIN8) | |
| | R: CCAGCATCATYTTTGCCACCAAGT | eugene3.00410102 | POPTR_0013s11520 | 3 |
| | | (NIN12) | (NIN12) | |
| NIN9/11 | F: GCTTCTCACMGCRGCATGCATMAA | fgenesh4_pg.C_LG_ | POPTR_0004s17480 | 3 |
| | | IV001415 (NIN9) | (NIN9) | |
| | R: TCYTCGAGTGCCACCRTRCCCAAA | gw1.IX.1371.1 (NIN11) | POPTR_0009s13160 | 3 |
| | | | (NIN11) | |
| SPS5 | F: TTGAAAGGAGYCTGTAGCAGTGCAAG | estExt_Genewise1_ | POPTR_0006s06300 | 4 |
| | R: TGAGACAAGCAGGCAGAGACTGTT | v1.C_1520214 | | |
| SPS6 | F: TTGAAAGGAGYCTGTAGCAGTGCAAG | eugene3.00181112 | POPTR_0025s00740 | 4 |
| | R: GAGCARCCAGGCAAAGACTTGTGA | | | |
| PAL1 | F: ATGTCTTTGCTTACGCCGATGACC | estExt_Genewise1v1.C_ | POPTR_0006s12870 | 5 |
| | R: TCATATGCTGCTCTTGCGCTCTCA | 280661 | | |
| PAL2 | F: AGAGAGTCCTGACAATGGGCTTCA | estExt_fgenesh4_pg.C_ | POPTR_0008s03810 | 5 |
| | R: GYTGGGTTGCCRTTCTCAAKTTCA | LG_VIII0293 | | |
| Elongation | F: AAGAGGACAAGAAGGCAGCA | estExt_fgenesh4_pg.C_ | POPTR_0001s23190 | 2 |
| factor 1-b | R: CTAACCGCCTTCTCCAACAC | LG_I1178 | | |
| Ubiquitin- | F: CTGAAGAAGGAGATGACARCMCCA | grail3.0013001001 | POPTR_0006s22210 | 2 |
| conjugating | R: GCATCCCTTCAACACAGTTTCAMG | | | |
| enzyme E2 | | | | |

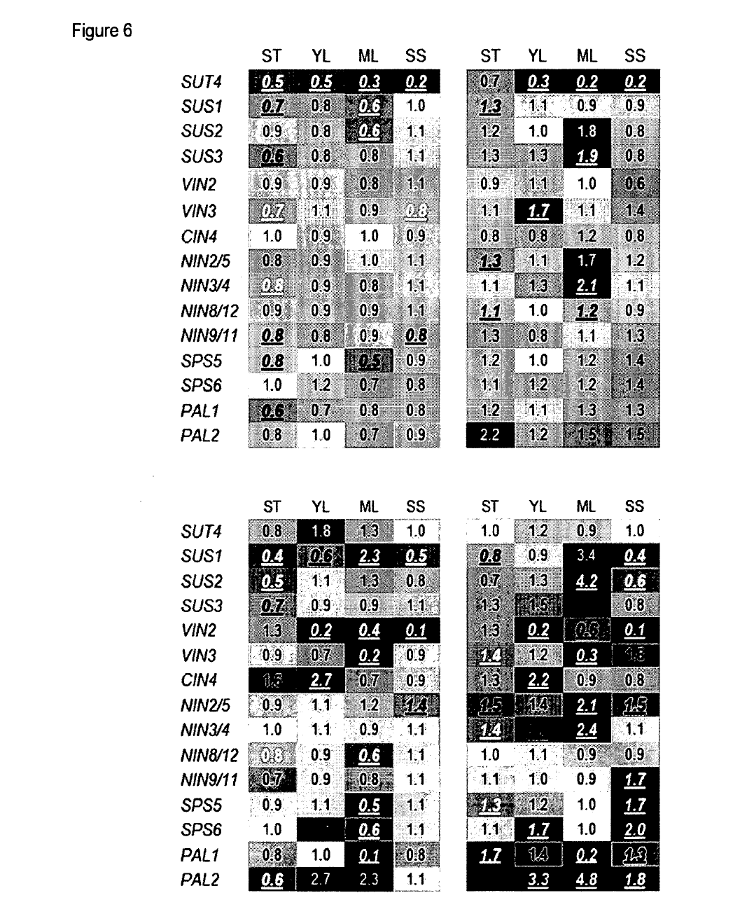

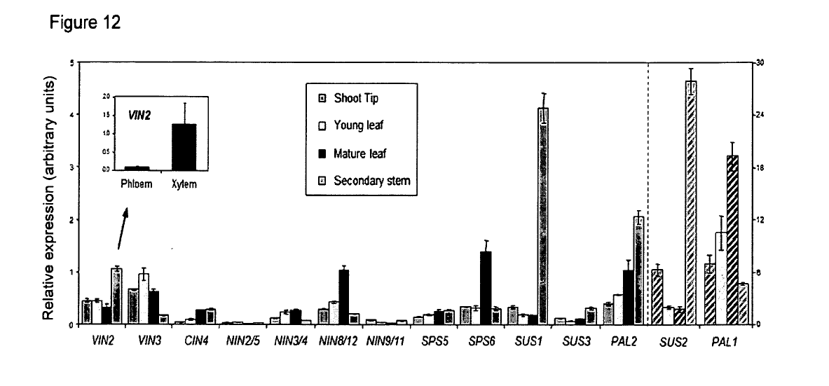

For real-time RT-PCR of low-abundant genes (not shown in FIGS. 6 and 12)

| | | | | |
|---|---|---|---|---|
| VIN1 | F: TTATCCGACGASGGCAATMTATGG | fgenesh4_pm.C_LG_ | POPTR_0003s12640 | 3 |
| | R: GGTACAGATGGATGCAAATTAGGT | III000407 | | |
| CIN1/2 | F: AGAAYTGCCATCWCATCYAGGGTT | gw1.XVI.2453.1 (CIN1) | POPTR_0016s07820 | 3 |
| | R: TTCATTGCGTGGATTCTCYCCC | gw1.XVI.2455.1 (CIN2) | POPTR_0609s00200 | 3 |
| CIN3 *** | F: AGAAYTGCCATCWCATCYAGGGTT | estExt_fgenesh4_pg.C_ | POPTR_0006s22710 | 3 |
| | R: GTACGGGACAATTTCATCATCCATTG | LG_VI1370 | | |
| CIN5 | F: TTTCGTAGACATGGATCCTCGCCA | eugene3.00061607 | POPTR_0006s24390 | 3 |
| | R: TGGCTTCCTTCGTTTCGTGGTAGA | | | |
| SPS1 | F: AGAYGTGGTGACAAAGGGTTCTGA | fgenesh4_pm.C_ | POPTR_0001s32500 | 4 |
| | R: TGAGACARGGGAAGGGTTAAGGGA | LG_I000988 | | |
| SPS2 | F: TACTGAAGGGAGTTGGCAGCAGTT | fgenesh4_pg.C_LG_ | POPTR_0018s01960 | 4 |
| | R: GCCTCTATCTCGGCAGATCGAAGAAA | XVIII000433 | | |
| SPS3 | F: AGAYGTGGTGACAAAGGGTTCTGA | fgenesh4_pg.C_ | POPTR_0017s08460 | 4 |
| | R: TGTTGGGCAGCTCTGTAGACCAAA | scaffold_88000008 | | |
| SPS4 | F: AGGCTTAGTGGAATACGGCAGTGA | gw1.XIII.3112.1 | POPTR_0013s09030 | 4 |
| | R: CCCATAGCCACTAGAGCTGCTGAGATA | | | |
| SUS4 | F: CAATCAAGGTGGCCCAGCAGAAAT | estExt_Genewise1_v1. | POPTR_0015s05540 | 2 |
| | R: GTAGATGCGTTGGAGACCAGTTGC | C_1220111 | | |
| SUS5 | F: CAATCAAGGTGGCCCAGCAGAAAT | eugene3.00120074 | POPTR_0012s03420 | 2 |
| | R: ATAGATGCGTTGAAGACCAGCTGC | | | |
| SUS6 | F: TGGATCCCGGACACTGGAATAAGT | eugene3.00440147 | POPTR_0017s02060 | 2 |
| | R: TCTGAGRCTGGTGTTTGASCTTCT | | | |

For various cloning

| | | | |
|---|---|---|---|
| SUT1/2 | F: ATGGAGAGTGGAGTTAGAAAAGAA | genomic DNA | 1 |
| | R: ACCAGTCACCAGTCTTGGAAGGAA | amplification | |
| SUT1 | F: ATGGAGAGTGGAGTTAGAAAAGAA | CDS cloning | 1 |
| | R: TCAAWGGAATGCARCASWASTACTGGTGGSAGC | | |
| SUT3 | F: ATGGAGAGTGGAGTTAGAAAAGAA | CDS cloning | 1 |
| | R: TCAATGGAATGCAKCAGTGACAGYC | | |
| SUT4 | F: CTAGCTAGCATGTCAGTCGCTAACCCAGAGCC | CDS cloning | 1 |
| | R: | | |
| | CGCTCGAGTCATGAGAAGACCATGGGCTTTTGAAC | | |
| SUT4 | F: GTCAGTCGCTAACCCAGAGCCACA | subcloning for | 1 |
| | R: TCATGAGAAGACCATGGGCTTTTGAAC | GFP tagging | |
| SUT5 | F: GCTCTAGATGGAGTCGGCRCCGATTCGGGTA | CDS cloning | 1 |
| | R: | | |
| | CCGCTCGAGTAGCATGCTCCTGTCCTTGACAATCA | | |
| SUT6 | F: ATGGAGTCGGCRCCGATTCGGGTA | CDS cloning | 1 |
| | R: TTAGCCAAAATGAAAACCACTGAAGGAACT | | |

* The SUT4 gene model predictions in the Poplar Genome v2.2 (Phytozome) was incorrect, spanning 3 partial gene models.
** The NIN5 gene model was missing from the Poplar Genome v2.2, and resides between POPTR_0005s01430 and POPTR_0005s01440.
References: 1, this example; 2, Payyavula et al., 2009, BMC PlantBiol., 9: 151; 3, Bocock et al., 2008, Planta 227: 565-576; 4, Geisler-Lee et al., 2006, Plant Physiol., 140: 946-962; 5, Kao et al., 2002, Plant Physiol., 130: 796-807.

### Gene Expression Analysis

Relative transcript abundance was determined by real-time RT-PCR and the SCT method as described (Tsai et al., (2006) New Phytol., 172, 47-62), using ubiquitin-conjugating enzyme E2 and elongation factor 1-β as housekeeping genes with three biological and two technical replicates. Expression data were visualized using the HeatMapperPlus tool (available online at bar.utoronto.ca/ntools/cgibin/ntools_heatmapper plus.cgi). Gene-specific primers are listed in Table 2. In situ localization of SUT and ribulose-1,5-bisphosphate carboxylase oxygenase (RuBisCO) transcripts in 10 μm thick paraffin-embedded tissue sections was carried out according to Kao et al., ((2002) Plant Physiol., 130, 796-807), except that hybridizations were extended to 36 hours at 40° C., and RNA probe concentration was increased to 1 ng μl⁻¹.

TABLE 2
List of sequences and gene models used in the phylogenetic analysis

| | | Gene locus or | Database source |
|---|---|---|---|
| Species | Gene name | accession no. | or reference |
| Arabidopsis | AtSUC1 | At1g71880 | 1 |
| thaliana | AtSUC2 | At1g22710 | 1 |
| | AtSUC3 | At2g02860 | 1 |
| | AtSUC4 | At1g09960 | 1 |
| | AtSUC5 | At1g71890 | 1 |
| | AtSUC6 | At5g43610 | 1 |
| | AtSUC7 | At1g66570 | 1 |
| | AtSUC8 | At2g14670 | 1 |
| | AtSUC9 | At5g06170 | 1 |
| Brachypodium | — | Bradi3g56740 | 2² |
| distachyon | — | Bradi4g00320 | 2 |
| | — | Bradi3g46790 | 2 |
| | — | Bradi1g73170 | 2 |
| Glycine max | — | Glyma02g08250 | 2 |
| | — | Glyma02g08260 | 2 |
| | — | Glyma02g38300 | 2 |
| | — | Glyma04g09460 | 2 |
| | — | Glyma08g40980 | 2 |
| | — | Glyma10g36200 | 2 |
| | — | Glyma16g27320 | 2 |
| | — | Glyma16g27330 | 2 |
| | — | Glyma16g27340 | 2 |
| | — | Glyma16g27350 | 2 |
| | — | Glyma18g15950 | 2 |
| Medicago | — | Medtr8g146560 | 2 |
| truncatula | — | Medtr5g076420 | 2 |
| Oryza sativa | OsSUT1 | LOC_Os03g07480, AAF90181 | 2, 3 |
| | OsSUT2 | LOC_Os12g44380, BAC67163 | 2, 3 |
| | OsSUT3 | LOC_Os10g26470, BAB68368 | 2, 3 |
| | OsSUT4 | LOC_Os02g58080, BAC67164 | 2, 3 |
| | OsSUT5 | LOC_Os02g36700, BAC67165 | 2, 3 |
| Ricinus communis | RcSUT2 | 29728.t000052 | 2 |
| | RcSUT3 | 29736.t000005 | 2 |
| | RcSUT4 | 30170.t000037 | 2 |
| Sorghum bicolor | — | Sb01g022430 | 2 |
| | — | Sb01g045720 | 2 |
| | — | Sb04g023860 | 2 |
| | — | Sb04g038030 | 2 |
| | — | Sb07g028120 | 2 |
| | — | Sb08g023310 | 2 |
| Vitis vinifera | VvSUT2 | GSVIVT00002307001, | 2 |
| | | AAL32020 | |
| | VvSUT11 | GSVIVT00015035001, | 2, 4 |
| | | AAF08329 | |
| | VvSUT12 | GSVIVT00037013001, | 2, 4 |
| | | AAF08330 | |
| | VvSUT27 | GSVIVT00002302001, | 2, 4 |
| | | AAF08331 | |
| Zea mays | ZmSUT1 | GRMZM2G034302, BAA83501 | 2, 5 |
| | ZmSUT2 | GRMZM2G145107, AAS91375 | 2, 5 |
| | ZmSUT3 | GRMZM2G083248, ACF86653 | 2, 5 |
| | ZmSUT4 | GRMZM2G307561, AAT51689 | 2, 5 |
| | ZmSUT5 | GRMZM2G081589, ACF85284 | 2, 5 |
| | ZmSUT6 | GRMZM2G106741, ACF85673 | 2, 5 |
| | ZmSUT7 | GRMZM2G087901 | 2 |
| Citrus sinensis | CsSUT1 | AAM29150 | 6 |
| | CsSUT2 | AAM29153 | 6 |
| Hordeum vulgare | HcSUT1 | CAB75882 | 7 |
| | HvSUT2 | CAB75881 | 7 |
| Juglans regia | JrSUT1 | AAU11810 | 8 |
| Lotus japonicus | LjSUT4 | CAD61275 | 9 |
| Phaseolus | PvSUT1 | ABB30164 | 10 |
| vulgaris | PvSUF1 | ABB30165 | 10 |
| Pisum sativum | PsSUF1 | ABB30163 | 10 |
| | PsSUF4 | ABB30162 | 10 |
| Plantago major | PmSUT1 | CAI59556 | 11 |
| | PmSUT2 | CAA53390 | 12 |
| | PmSUT3 | CAD58887 | 13 |
| Solanum | SlSUT1/LeSUT1 | CAA57726 | 14 |
| lycopersicum | SlSUT2/LeSUT2 | AAG12987 | 14 |
| (Lycopersicon | SlSUT4/LeSUT4 | AAG09270 | 14 |
| esculentum) | | | |
| Solanum | StSUT1 | CAA48915 | 15 |
| tuberosum | StSUT4 | AAG25923 | 16 |

Database source or reference:
1, TAIR (TAIR database refers to The Arabidopsis Information Resource, available through the world wide web);
2, Phytozome (Phytozome database refers to joint project of the Department of Energy's Joint Genome Institute and the Center for Integrative Genomics. It is available through the world wide web);
3, Aoki, et al., (2003) Plant Cell Physiol., 44, 223-232;
4, Barth, 2003, Plant Cell, 15: 1375-1385;
5, Braun and Slewinski, 2009, Plant Physiol., 149: 71-81;
6, Li et al., 2003, Biochem. Biophys. Res. Commun., 306: 402-407;
7, Endler et al., 2006, Plant Physiol., 141: 196-207;
8, Decourteix et al., 2006, Plant Cell Environ., 29: 36-47;
9, Flemetakis et al., 2003, J. Exp. Bot., 54: 1789-1791;
10, Zhou et al., (2007) Plant J., 49, 750-764;
11, Lauterbach et al., 2007, Plant Biol., 9: 357-365;
12, Gahrtz et al., 1994, Plant J., 6: 697-706;
13, Barth et al., 2003, Plant Cell, 15: 1375-1385;
14, Barker et al., 2000, Plant Cell, 12: 1153-1164;
15, Riesmeier et al., 1993, Plant Cell, 5: 1591-1598;
16, Weise et al., 2000, Plant Cell, 12: 1345-1355.

### Yeast Complementation

Full length coding sequences of PtaSUT3, PtaSUT4 and PtaSUT5 were cloned into the PDR196 vector (Rentsch et al., (1995) FEBS Lett., 370, 264-268) obtained from the Arabidopsis Biological Resource Center. The resultant plasmids were transformed into the SUSY7/ura3 strain (Riesmeier et al., (1992) EMBO J., 11, 4705-4713) using lithium acetate (Gietz and Woods, (2002) Methods Enzymol., 350, 87-96). Transformants were selected on uracil-deficient medium and confirmed by PCR. Transformed and untransformed mutants were cultured on yeast nitrogen base without amino acids (Difco), supplemented with either 2% glucose or 2% sucrose as sole carbon source for 3 days.

### Subcellular Localization

PtaSUT4 coding sequence was PCR-amplified and cloned into pCX-DG vector for GFP-tagging (Chen et al., (2009) Plant Physiol., 150, 1111-1121). The resultant GFP:PtaSUT4 fusion plasmid was transformed into Agrobacterium tumefaciens strain C58/pMP90 using the freeze-thaw method (Holsters et al., (1978) Mol. Gen. Genet., 163, 181-187). A second A. tumefaciens strain AGL1 harboring the viral silencing suppressor p19 from tomato bushy stunt virus in the pKyLx vector (Schardl et al., (1987) Gene, 61, 1-11, obtained from H. Scholthof, Texas A&M University) and used to enhance transient expression of GFP:PtaSUT4 (Voinnet et al., Plant J., 33, 949-956). The two Agrobacterium strains were mixed in a 1:1 ratio for coinfiltration of Nicotiana benthamiana leaves according to Wydro et al., ((2006) Acta Biochim. Pol., 53, 289-298). In filtration with the p19 strain alone serves as a negative control. Four days after infiltration, epidermis was removed (Wu et al., (2009) Plant Methods, 5, 10), and mesophyll protoplasts released using 1% cellulose-R10 and 0.4% Macerozyme-R10 (Yoo et al., (2007) Nat. Protoc., 2, 1565-1572). Protoplasts were imaged using a Leica SP5 confocal microscope.

### Populus Transformation

A 200 bp PtaSUT4 3′-UTR fragment was amplified from the 3′-region of the Populus cDNA and cloned into an XcmI-digested pGFPm-T vector (Luo et al., (2008) Biotechnol. Lett., 30, 1271-1274) and transformed into TOP10 competent Escherichia coli cells. Subcloning of the SUT4 fragment and development of an inverted repeat in the pGSA1285 binary vector backbone (available online at chromdb.org) was performed as described (Luo et al., (2008) Biotechnol. Lett., 30, 1271-1274). The construct was transformed into A. tumefaciens strain C58/pMP90 as above. Transformation of P. tremula x alba clone 717-1B4 was performed as described (Meilan and Ma, (2006) In: Methods in Molecular Biology, vol 34, Agrobacterium Protocols (Wang, K. ed: Humana Press, pp. 143-151).

### Hydroponic Nitrogen Treatment Studies and Biomass Measurements

Plants 10-15 cm in height were distributed into small, 10-L hydroponics tubs. After growth in nutrient solution with 2.5 mM total nitrogen (N-replete) for 8 days, plants were subjected to N-replete or N-limiting (0.125 mM total N) treatments. In all cases, N was supplied as a molar ratio of NO3-:NH4+=4:1, and the nutrient solution was changed every 2 days to maintain the N treatment levels. The plants were harvested after 2 weeks. No premature leaf senescence or terminal bud dormancy was observed during the experimental period. For each treatment, 8-10 individual plants were used. Growth was monitored during the experimental period. A proxy estimate of leaf area was calculated for each plant by multiplying midrib length by maximum width, and summing the estimates of nine individual leaves between LPI-0 through LPI-8. Ten leaf discs (0.6 cm diameter) from LPI-5 were oven-dried and weighed to determine whether there were genotype differences in specific leaf mass. A proxy estimate of stem volume was calculated by multiplying stem height by stem diameter at mid height.

### Sugar and Starch Quantification

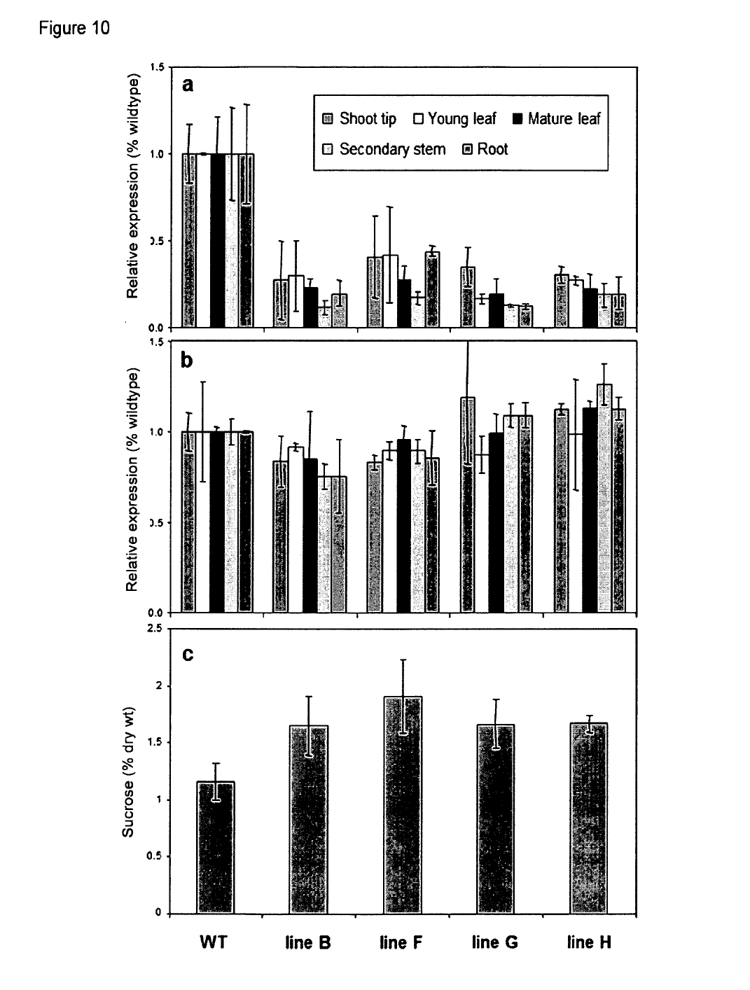

Sucrose level was estimated using the high-performance thin-layer chromatography (HPTLC) method of Sherma and Zulick ((1996) Acta Chromatographica, 6, 7-13) in preliminary screening (FIG. 10). In subsequent analyses, sucrose, glucose and fructose were estimated using the GC-MS metabolic profiling protocol of Jeong et al. (2004) with modifications. Ten mg freeze-dried tissue was extracted twice in 600 μl methanol:water:chloroform (3.2:2.0:1.6). The abundant polar phenolics and their glycosides were removed from the aqueous, sugar-containing phase by solid-phase extraction using the Advanta™ resin (Applied Separations) (Sirvent et al. 2004). Following methoximation and derivitization (Jeong et al., 2004), the equivalent of 0.7 μg of freeze-dried tissue was injected for analysis of sugars against standard curves of glucose, fructose and sucrose. Conditions for GC-MS were as described (Jeong et al., 2004). Starch was estimated by an enzymatic method (Chow and Landhausser, (2004) Tree Physiol., 24, 1129-1136).

### Phenolic Glycoside and Condensed Tannin Analysis

Freeze-dried samples were analyzed for phenolic glycosides (PGs) and condensed tannins (CTs) as described (Harding et al., (2005) Tree Physiol., 25, 1475-1486) but without the alkaline hydrolysis step. PGs separated by high-performance thin-layer chromatography (HPTLC) were scanned at a wavelength of 270 nm using a TLC scanner (Camag), and quantified using authentic salicin, and salicortin standards (provided by Richard Lindroth, University of Wisconsin-Madison). Soluble and residue-bound CTs were estimated according to Porter et al. ((1986) Phytochemistry, 25, 223-230), using a standard curve based on purified aspen leaf CT.

### Statistics

Statistical calculations were performed using SigmaStat 3.5 (Systat Software). The students t-test was used to determine statistical significance of transgenic effects.

### Results

### The Populus SUT Gene Family

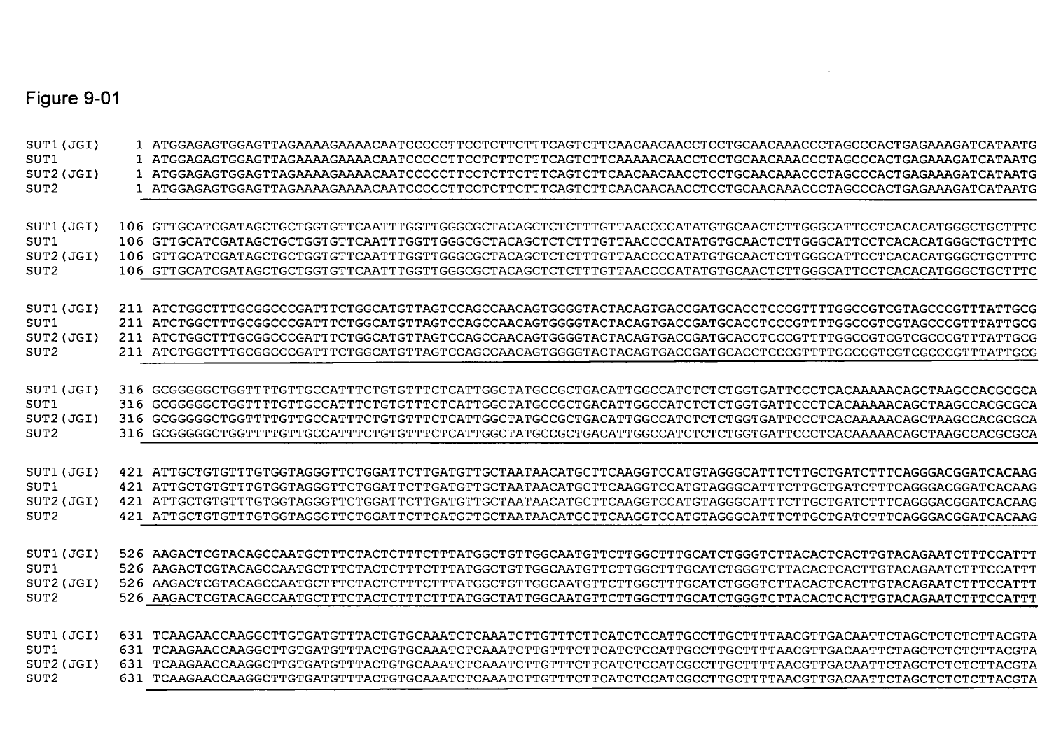

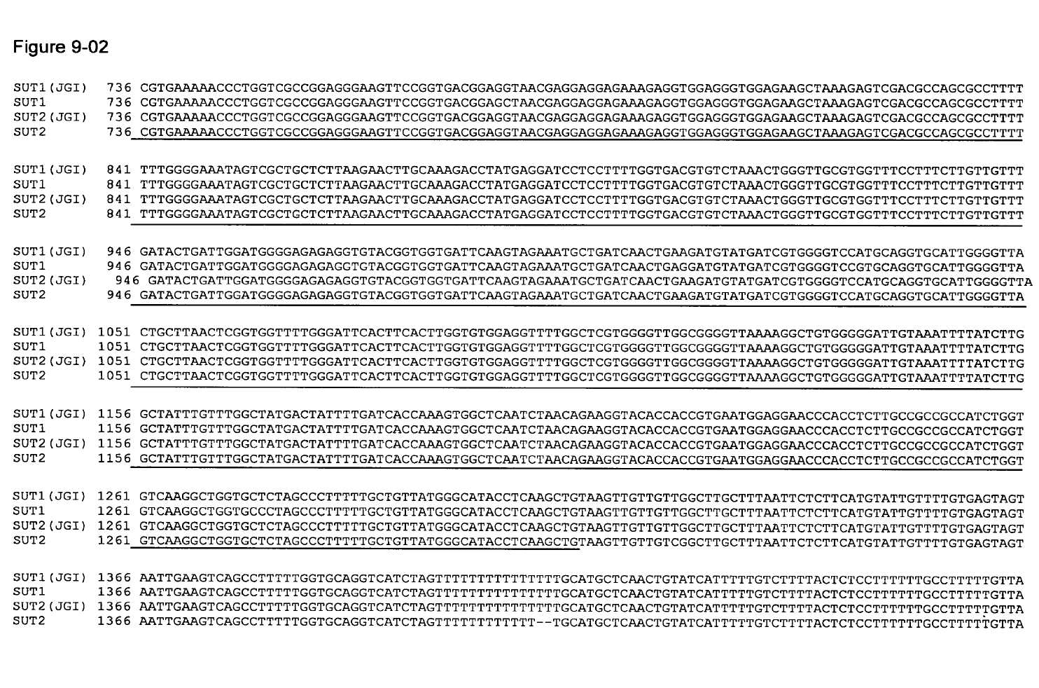

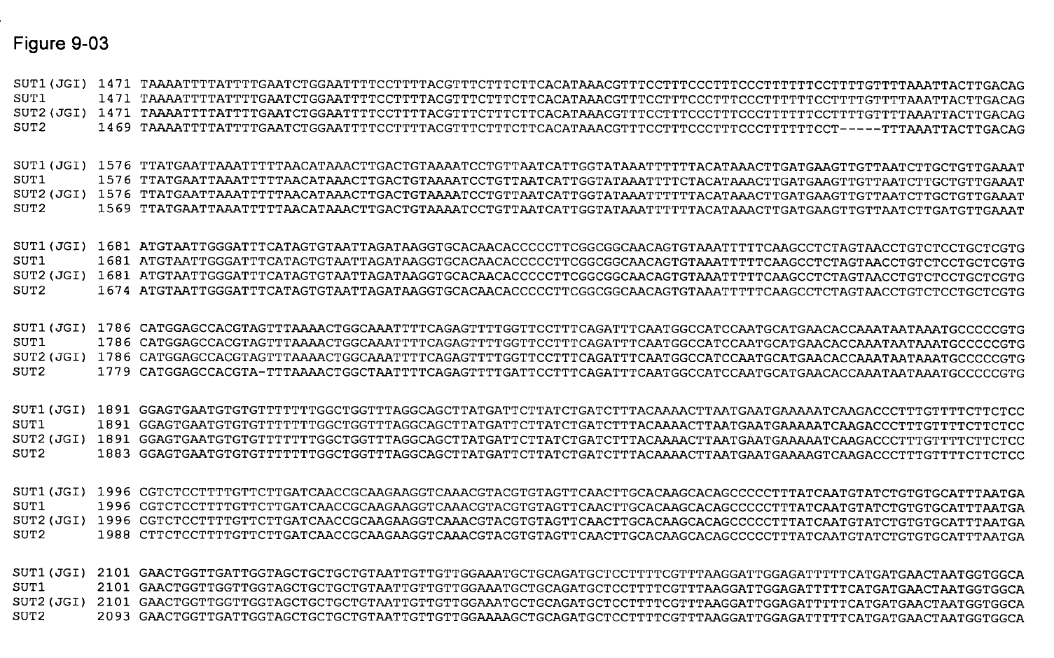

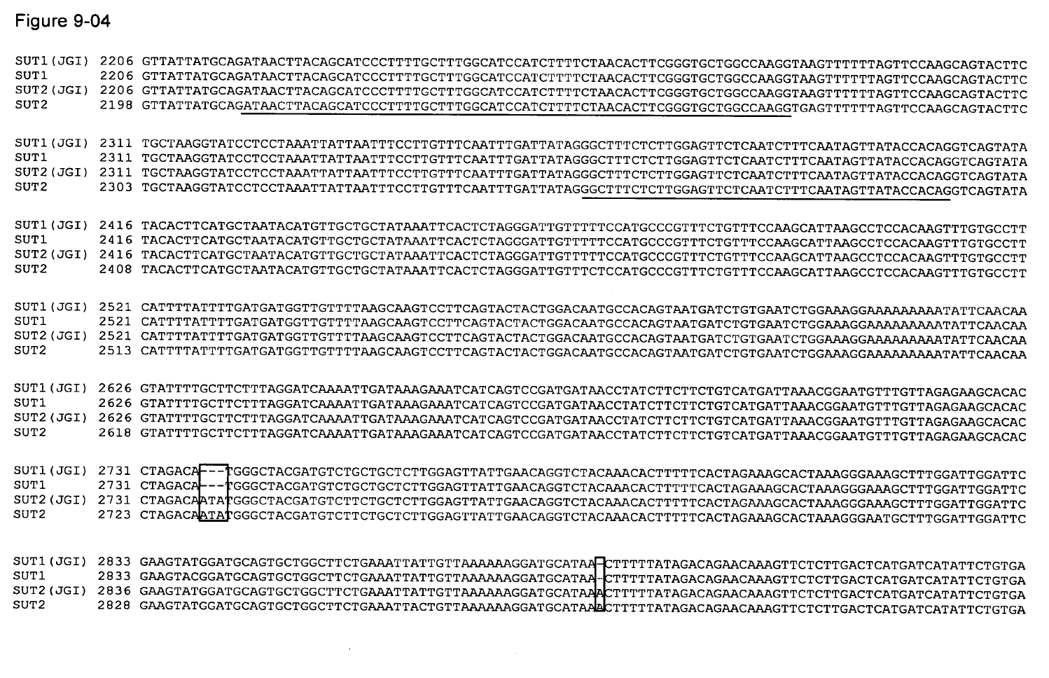

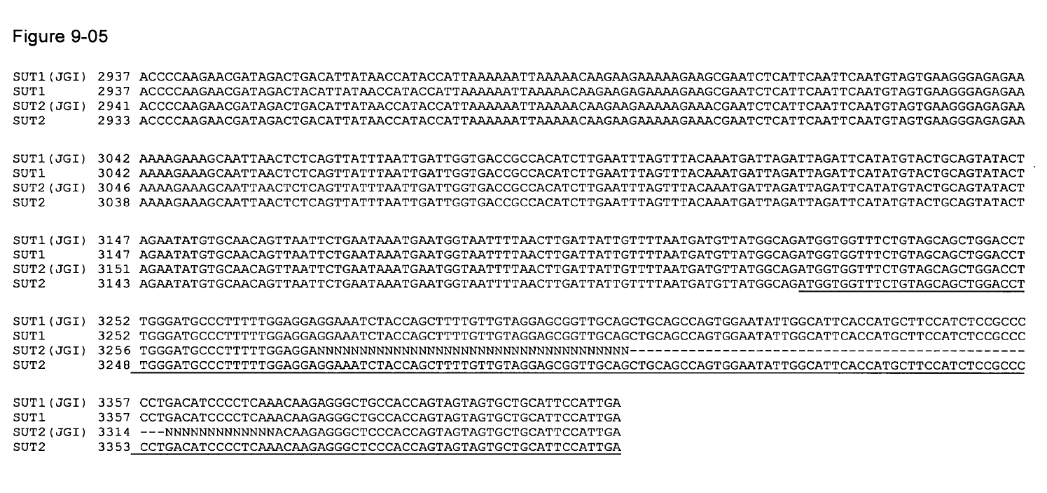

Six SUT genes were initially identified in the P. trichocarpa (Nisqually-1) genome v1.1 (Tuskan et al., (2006) Science, 313, 1596-1604). SUT1 and SUT2 are nearly identical and are located on scaffolds not yet assembled into the 19 linkage groups. Full-length coding sequences for all except SUT2 were cloned from gray poplar (P. tremula x alba) by RT-PCR. Repeated attempts to clone SUT2 from a range of tissues or from genomic DNA yielded only SUT1. Full-length SUT1 and SUT2 sequences were eventually cloned from P. trichocarpa (Nisqually-1) genomic DNA (GenBank accession numbers HM749896-HM749897). The two sequences differ chiefly by two small but diagnostic intronic indels (FIG. 9). Both gene models have recently been mapped to a single locus (Poptr_{—}0013s11950) in the updated poplar genome assembly v2.0 (www.phytozome.net). We concluded that these two sequences represent allelic variants of SUT1, and that the Populus SUT family contains five members. Hereafter, we refer to the gray poplar genes as PtaSUT1, PtaSUT3, PtaSUT4, PtaSUT5, and PtaSUT6 (GenBank accession numbers HM749898-HM749902). Four of the genes form two pairs of highly similar (>90%) sequences with identical gene structure: PtaSUT1 with PtaSUT3, and PtaSUT5 with PtaSUT6 (Table 3). Their P. trichocarpa orthologs are located on homeologous chromosome regions, resulting from “salicoid” genome-wide duplication events (Tuskan et al., (2006) Science, 313, 1596-1604). The fifth gene, PtaSUT4, constitutes a third group. The between-group amino acid sequence similarity is less than 64%. SUT5 and SUT6 contain fourteen exons, about three times the number in the other SUT genes (4 or 5 exons), and their predicted polypeptides are relatively large, ˜64 kDa vs. 55-57 kDa for the other SUT proteins. PtaSUT5 and PtaSUT6 have acidic theoretical pI's of ˜6.1, while other isoforms have theoretical pI's>9.0 (Table 3).

TABLE 3
Characteristics of the Populus SUT sequences

| | | | | | % Similarity | | | |
|---|---|---|---|---|---|---|---|---|
| | Size | | | Exon | (nucleotide/amino acid to | | | |
| | a.a. | kDa | pI | no. | SUT3 | SUT4 | SUT5 | SUT6 |
| SUT1 | 535 | 56.9 | 9.32 | 4 | 90/93 | 56/64 | 46/57 | 46/56 |
| SUT3 | 532 | 56.7 | 9.19 | 4 | — | 56/64 | 46/57 | 46/56 |
| SUT4 | 510 | 55.4 | 9.19 | 5 | — | — | 46/55 | 45/54 |
| SUT5 | 597 | 64.3 | 6.10 | 14 | — | — | — | 91/92 |
| SUT6 | 601 | 64.6 | 6.05 | 14 | — | — | — | — |

### Phylogenetic Analysis of SUT Proteins

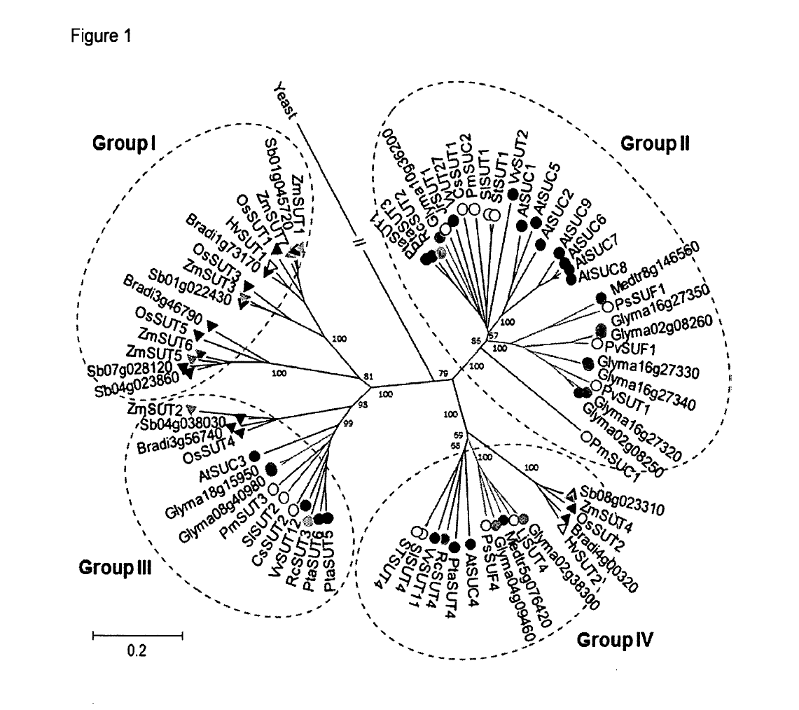

Plant SUT proteins were originally classified into three phylogenetic groups according to Aoki et al. (2003, Plant Cell Physiol., 44, 223-232), and later into four distinct classes by Sauer ((2007) FEES Lett., 581, 2309-2317). An expanded phylogenetic analysis of SUT orthologs from 10 sequenced genomes (six dicots and four monocots) supports the latter classification (FIG. 1, Table 2), with Group I and Group II representing monocot- and dicot-specific branches, respectively. PtaSUT1 and PtaSUT3 belong to Group II, and cluster with SUT isoforms from other perennial species (e.g., Ricinus, Vitis, Juglans and Citrus). This subclade also includes SUT proteins from herbaceous species, but is distinct from the Arabidopsis- and legume-specific subclades (FIG. 1). PtaSUT5 and PtaSUT6 fall into the dicot subclade of Group III, clustering with other perennial SUT orthologs. Group IV also forms monocot- and dicot-specific subclades, with the dicot group divided into a legume-specific branch and a non-legume branch that includes PtaSUT4. In dicots, plasma membrane-localized SUT proteins that mediate apoplastic loading generally fall into Group II, as for example, Arabidopsis AtSUC2 (Chandran et al., (2003) J. Biol. Chem., 278, 44320-44325) and tomato (Solanum lycopersicum) S1SUT1 (Hackel et al., (2006) Plant J., 45, 180-192). Tonoplast-localized isoforms are found in Group IV, exemplified by Arabidopsis AtSUC4 (Endler et al., (2006) Plant Physiol., 141, 196-207) and lotus (Lotus japonicus) LjSUT4 (Reinderset al., (2008) Plant Mol. Biol., 68, 289-299). The function of Group III SUTs remains less clear (Braun and Slewinski, (2009) Plant Physiol., 149, 71-81).

### Expression of PtaSUT Genes in Populus

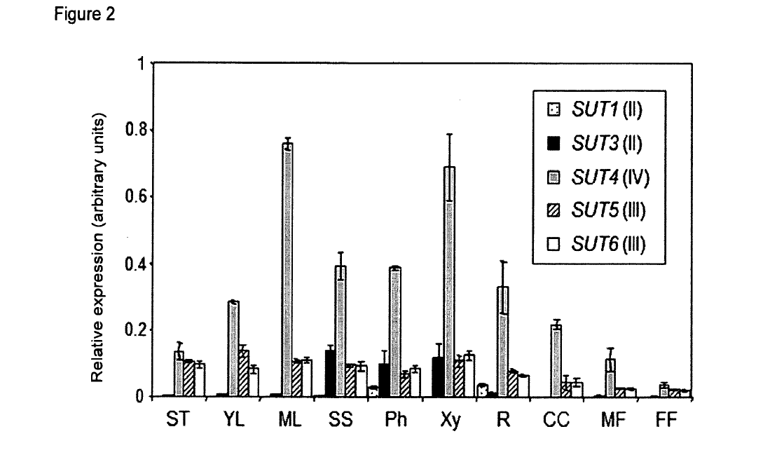

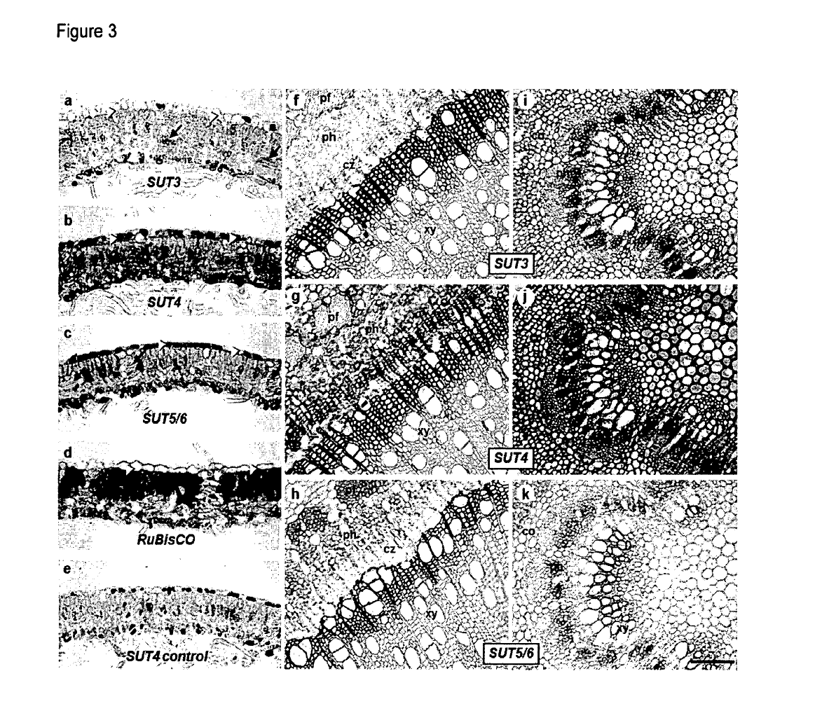

The Group IV transporter PtaSUT4 was ubiquitously expressed based on QPCR, and its transcripts were more abundant than those of the other PtaSUT genes in every tissue examined (FIG. 2). PtaSUT4 transcript levels were highest in mature source leaf (ML) and developing xylem. Transcripts of the Group III members, PtaSUT5 and PtaSUT6, were also readily detected, and at similar levels across all tissues sampled from greenhouse plants. Their level was much lower than that of PtaSUT4 in most tissues, but was comparable in shoot tips (ST). Transcripts of Group II PtaSUT1 and PtaSUT3 were detected mainly in roots and stems, respectively. PtaSUT3, PtaSUT4 and PtaSUT5, were selected for in situ transcript localization (FIG. 3). With their strong sequence and expression-pattern similarity, PtaSUT5 and PtaSUT6 transcripts contributed equally to in situ hybridization signal. PtaSUT1 and PtaSUT3 sequences are also highly similar; however, PtaSUT3 was the predominantly expressed Group II member in shoot organs.

PtaSUT4 transcripts were present in epidermal cells, spongy mesophyll and lower layers of the palisade mesophyll in lamina of source leaves, but not in the upper palisade layer (FIG. 3b). Hybridization was also evident in minor phloem traces (red arrows) in source leaves. PtaSUT3 transcripts were observed in minor veins of source leaf lamina, but were barely detected in the mesophyll (FIG. 3a). PtaSUT5/6 expression was detected in all cell types, but at lower levels than PtaSUT4 (FIG. 3c). By comparison, RuBisCO transcripts were abundant in all mesophyll layers, but not in vascular traces (FIG. 3d). In stems, PtaSUT4 transcripts were readily detected in mesophyll and conducting cells of phloem, and in ray parenchyma, fibers and vessels of the secondary xylem (FIG. 3g). Vascular cambium and dividing xylem cells also yielded clear hybridization signals. In conducting cells of primary stem internodes, PtaSUT4 signals were as strong, and in the cortical mesophyll, stronger than in secondary stem internodes (FIG. 3j). There was strong PtaSUT3 hybridization in xylem vessel and fiber cells, but not in adjacent phloem tissues (FIG. 3f). Both PtaSUT3 and PtaSUT5/6 were more preferentially expressed in the xylem ray parenchyma than was PtaSUT4. (FIG. 3f, h). Weak PtaSUT5/6 signal was also detected in phloem tissues, including fibers where PtaSUT3 and PtaSUT4 transcripts were not detected. In contrast to PtaSUT4, hybridization signals of PtaSUT3 and PtaSUT5/6 were weaker in the primary internodes than in secondary internodes (FIG. 3f-k).

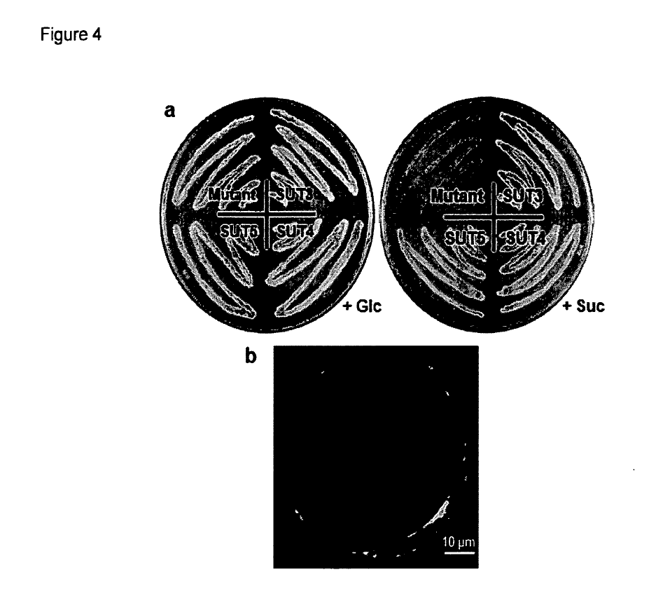

PtaSUT4 exhibits sucrose transport activity and is localized to the tonoplast Function of the Populus SUT proteins was verified using the Saccharomyces cerevisiae SUSY7/ura3 mutant strain. This mutant is deficient in sucrose transport activity and unable to grow on media containing sucrose as the sole carbon source (Riesmeier et al., (1992) EMBO J., 11, 4705-4713). Ectopic expression of PtaSUT3, PtaSUT4 and PtaSUT5 complemented the mutant phenotype on sucrosecontaining media (FIG. 4a). Active sucrose transporter rather that passive sucrose facilitator function is supported for all three Populus isoforms by their capacity to rescue growth of the mutant on media supplemented with only 2% sucrose (Zhou et al., (2007) Plant J., 49, 750-764). Unlike the Group II and Group III members, PtaSUT4 was strongly expressed in both source and sink organs. As a first step toward detailed functional analysis, membrane-targeting of PtaSUT4 protein was determined. Group IV SUT proteins are localized to the tonoplast in several species (Endler et al., (2006) Plant Physiol., 141, 196-207, Reinderset al., (2008) Plant Mol. Biol., 68, 289-299), but have been found in the plasma membrane in potato and, according to one proteomic survey, Populus (Chincinska et al., (2008) Plant Physiol., 146, 515-528, Nilsson et al., (2010) Mol. Cell. Proteomics, 9, 368-387). Therefore, to confirm localization of PtaSUT4, N benthamiana leaves were infiltrated with Agrobacterium carrying the GFP-PtaSUT4 construct and protoplasts were isolated for visualization of GFP targeting. Targeting of PtaSUT4 protein to the tonoplast was supported by localized GFP signal along the perimeter of a large structure, presumably the tonoplast surrounding the major vacuole, and interior to the chloroplasts (FIG. 4b).

Transgenic manipulation of PtaSUT4 by RNAi altered biomass partitioning

The comparatively high transcript levels of PtaSUT4 in all tissues analyzed, and the near absence of Group II transporter transcripts in exporting leaves suggests that PtaSUT4 protein has a larger role in sucrose efflux than its Group IV ortholog does in apoplastic species where Group II SUTs predominate. In an effort to determine whether sucrose export depends on PtaSUT4 function in Populus, RNAi-mediated gene silencing was performed. PtaSUT4 transcript levels were reduced by 50-90% in different tissues of four independent transgenic poplar lines (FIG. 10a). Transcript levels of PtaSUT5 were not systematically affected in transgenic plants, confirming specificity of the PtaSUT4 silencing (FIG. 10b). Based on these results, and on preliminary measurements showing that all four lines exhibited higher source-leaf sucrose contents than wild-type (WT) controls (FIG. 10c), transgenic line G exhibiting the strongest overall suppression of PtaSUT4 was propagated for further analysis.

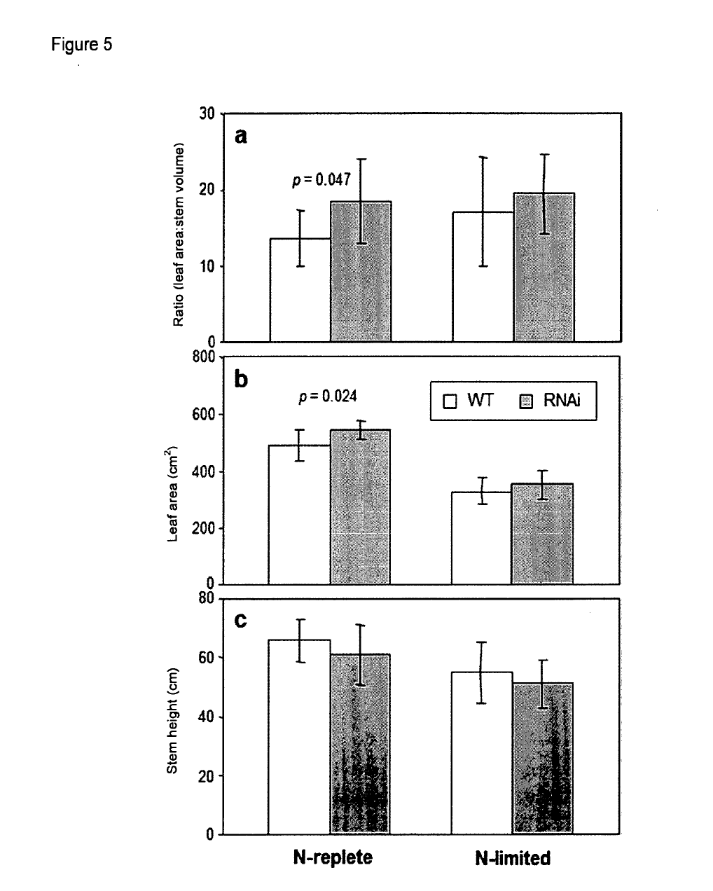

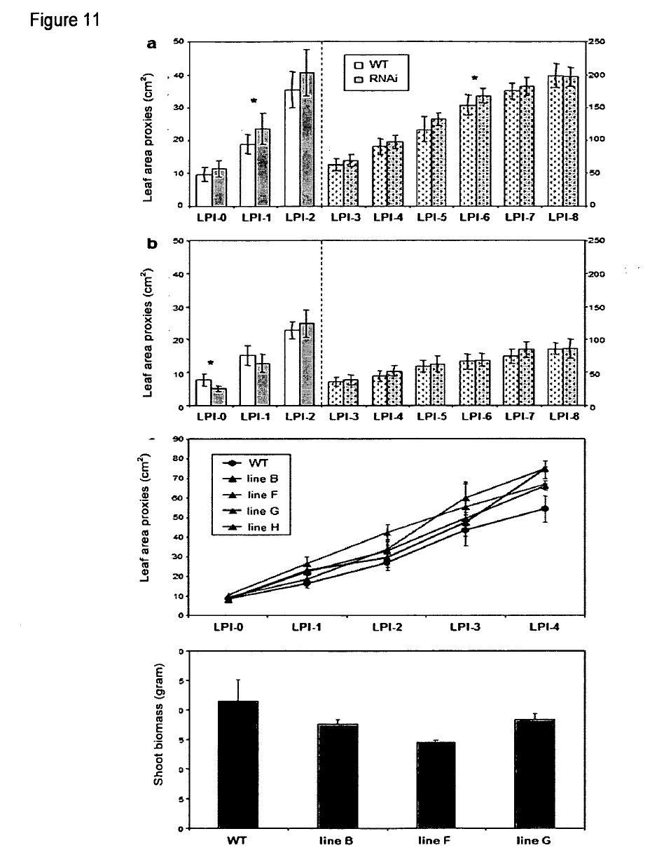

Plant growth was monitored under hydroponic conditions where N supply could be conveniently and uniformly varied to alter plant-wide patterns of carbon use. Suppression of PtaSUT4 had no obvious effect on plant appearance or growth. However, the ratio of leaf area to stem volume proxies was greater in transgenic than WT plants under the two N regimes tested (FIG. 5a). Leaf and stem dry masses per unit area and volume, respectively, were identical in transgenic and WT plants. The difference in leaf:stem ratio was consistent with shorter stem height and greater summed leaf area (in the leaf expansion zone) of transgenics compared to WT (FIG. 5b,c). In N-replete plants, the leaf area differential was largest in non-exporting leaves, LPI-0 and LPI-1 (FIG. 11a), smaller in source leaves reaching full size, and not evident in fully mature source leaves (LPI-8). During N-limitation, importing leaves were smaller in the transgenics, but sink-source transitional and source leaves expanded to a larger size in transgenic than WT plants (FIG. 11b). The RNAi effect on early leaf expansion and shoot growth of N-replete plants was robust, and similar trends were observed in all four transgenic lines when they were grown in soil (FIG. 11 c, d).

### RNAi Perturbed Carbohydrate and Phenylpropanoid Gene Expression

Transcript levels of SUT4 were substantially reduced in all shoot organs of the transgenic plants (FIG. 6a). Transcript abundance of various carbohydrate-active enzyme (CAZyme)-encoding genes, including sucrose synthase (SUS), invertase, and sucrose phosphate synthase (SPS) members, was also altered. During N-replete growth, the predominant SUS (SUS2) and SPS(SPS6) isoforms exhibited lower transcript levels in MI, of transgenic than of WT plants (FIG. 6a, FIG. 12). The effects on SUS and SPS transcript abundance in secondary stems (SS) with well-developed phloem and xylem, were generally less striking than in leaves (FIG. 6a). Expression of several other CAZyme genes was little affected in the transgenic plants under N-replete growth.

Use of two N regimes revealed additional differences in gene regulation between the WT and transgenic plants. Genes that were comparatively under-expressed in source leaves of transgenic plants during N-replete growth were comparatively over-expressed in transgenic plants during N-limited growth (FIG. 6a,b). This was especially evident for SUS2, a CAZyme gene with relatively high transcript levels in source leaves (FIG. 12). Transcript levels of several of the cytosolic neutral invertases (NIN) were also higher in source leaves of transgenic than WT plants during N-limited growth (FIG. 6b). SPS6 transcript levels were less affected and were relatively stable in transgenic source leaves regardless of N-status (FIG. 6c,d). A general effect of reduced plant N-status in both control and transgenics was that transcript levels of well-expressed vacuolar invertases VIN2 and VIN3 decreased sharply in secondary stems and source leaves, respectively (FIGS. 6c,d and 12). The N-status-modulated changes in source leaf transcript levels of SUS1, SUS2, and NIN3/4 were therefore larger, relative to VINs, in transgenic than WT plants (FIG. 6c,d). Overall, and in contrast to the situation in N-replete plants, transcript levels of the CAZyme genes increased in leaves relative to secondary stems in N-limited transgenic plants (FIG. 6b,d). Compared to the case with N-replete plants, RNAi effects on SUS and SPS transcript abundance in secondary stems were more pronounced in N-limited plants, trending toward up-regulation for SPSs and down-regulation for SUSs (FIG. 6a,b).

RNAi Effects on Sucrose Homeostastis Differed with N-Status

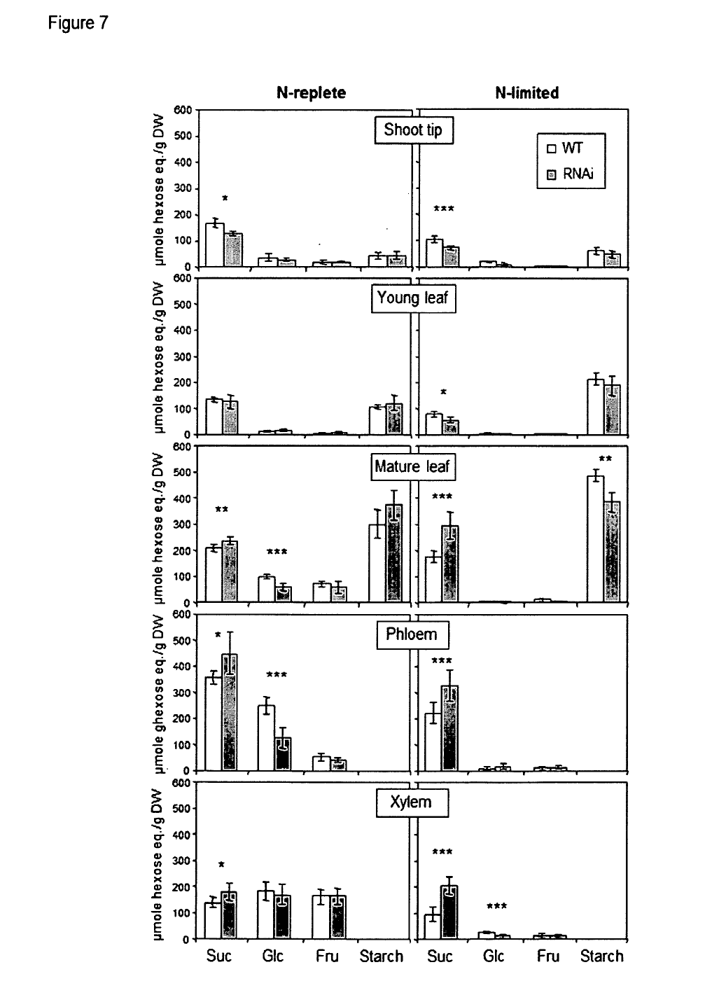

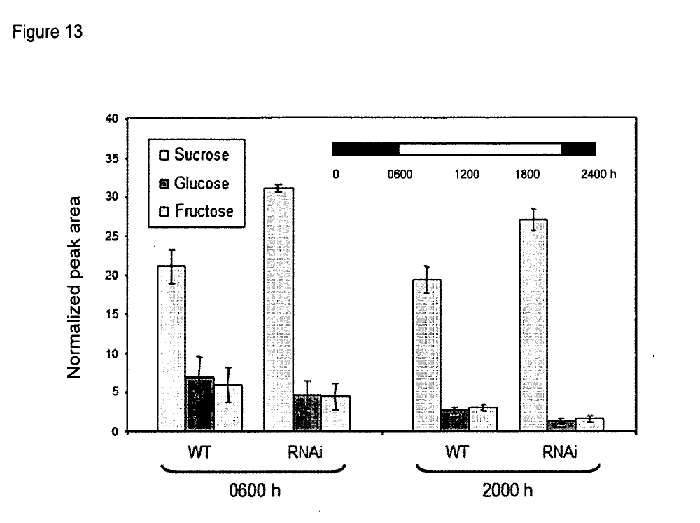

Mid-day sucrose contents were higher in fully expanded source leaves, phloem and xylem of transgenics than WT (FIG. 7). At the same time, glucose levels were reduced in those organs of transgenic plants, and this resulted in relatively high sucrose-to-hexose ratios in the transgenic organs (Table 4). Sugar levels were not affected in sink-source transitional leaves (YL) in N-replete plants, but were reduced in ST composed primarily of terminal sink leaves. Starch abundance trended higher in leaves of transgenic plants under N-replete conditions (FIG. 7). Because SUT transcript levels are lower in Populus at mid-day than at other time points (Wilkins et al., (2009) Plant J., 60, 703-715), we conducted a separate experiment to examine RNAi effects on source leaf sucrose levels at the end of the 9-h dark period (0600 h) and at the end of the 15-h light period (2100 h). In both genotypes, sucrose levels at dusk were lower than they were pre-dawn (FIG. 13). At both time points, sucrose levels were higher, and hexose levels were lower in source leaves of transgenic than WT plants.

TABLE 4
Molar ratios of sucrose to hexose (glucose + fructose).

| | High N | | | Low N | | |
|---|---|---|---|---|---|---|
| | Control | RANi | p value | Control | RANi | p value |
| Shoot tip | 1.69 ± 0.72 | 1.45 ± 0.32 | NS | 2.27 ± 0.18 | 6.04 ± 1.02 | NS |
| Young | 3.94 ± 1.12 | 2.56 ± 0.42 | 0.027 | 5.35 ± 0.87 | 9.45 ± 0.76 | NS |
| leaf | | | | | | |
| Mature | 0.60 ± 0.04 | 1.04 ± 0.24 | <0.001 | 5.61 ± 1.94 | 58.16 ± 7.12 | <0.001 |
| leaf | | | | | | |
| Xylem | 0.21 ± 0.03 | 0.28 ± 0.04 | 0.006 | 1.23 ± 0.28 | 10.24 ± 2.10 | 0.006 |
| Phloem | 0.59 ± 0.06 | 1.45 ± 0.63 | <0.001 | 6.58 ± 2.71 | 10.98 ± 2.37 | NS |

Means and standard deviations of the molar ratios were obtained from n = 4 (shoot tip, each sample was pooled from two plants) to 8 biological replicates.
Statistical significance (p values) of the differences between genotype means was determined by the Students t-test.
NS, not significant.

Growth under N-limiting conditions caused striking changes in the carbohydrate differentials between transgenic and WT plants. Low N-status caused sucrose levels to be higher in source leaf and xylem of transgenic plants but lower in WT (FIG. 7). Low N-status also led to significant reductions of leaf and xylem hexoses in both transgenic and WT plants. The differential in sucrose-to-hexose ratios between transgenics and WT was 10-fold greater, on a molar basis, than the differential observed between N-replete plants (Table 4). The generally larger variation among N-stressed plants than among N-replete plants might have diminished the statistical significance in many cases, but the trend of higher sucrose-to-hexose ratios in transgenic tissues was consistent (FIG. 7 and Table 4). Starch abundance was higher in WT leaves under N-limiting conditions.

### Phenylpropanoid Homeostasis and Gene Expression was Altered in RNAi Plants

Because of the quantitative importance of glycosylated phenylpropanoid products, such as PGs, to the carbon budget of Populus (Harding et al., (2009) J. Exp. Bot., 60, 3443-3452, Lindroth and Hwang, (1996) Biochem. Syst. Ecol., 24, 357-364), and because of the established link between sugars and phenylpropanoid metabolism (Ehness et al., (1997) Plant Cell, 9, 1825-1841, Essmann et al., (2008) Plant Physiol., 147, 1288-1299, Rolland et al., (2006) Annu. Rev. Plant Biol., 57, 675-709), transcript levels of phenylalanine ammonia-lyase (PAL) genes were analyzed. We have previously reported that PAL1 is strongly expressed in mesophyll cells where non-structural phenylpropanoids normally accumulate, whereas PAL2 is mainly expressed in vascular tissues of Populus (Kao et al., (2002) Plant Physiol., 130, 796-807). In the current study, transcript levels of PAL1 were lower in N-replete transgenic plants than in WT (FIG. 6a,b). Transcript levels of PAL2 increased during N-limited growth in leaves of both genotypes, but the increase was more striking in the transgenics (FIG. 6c,d). It was generally evident, as for CAZyme genes, that PAL gene expression was differentially modulated by N-status in transgenic and WT plants (FIG. 6).

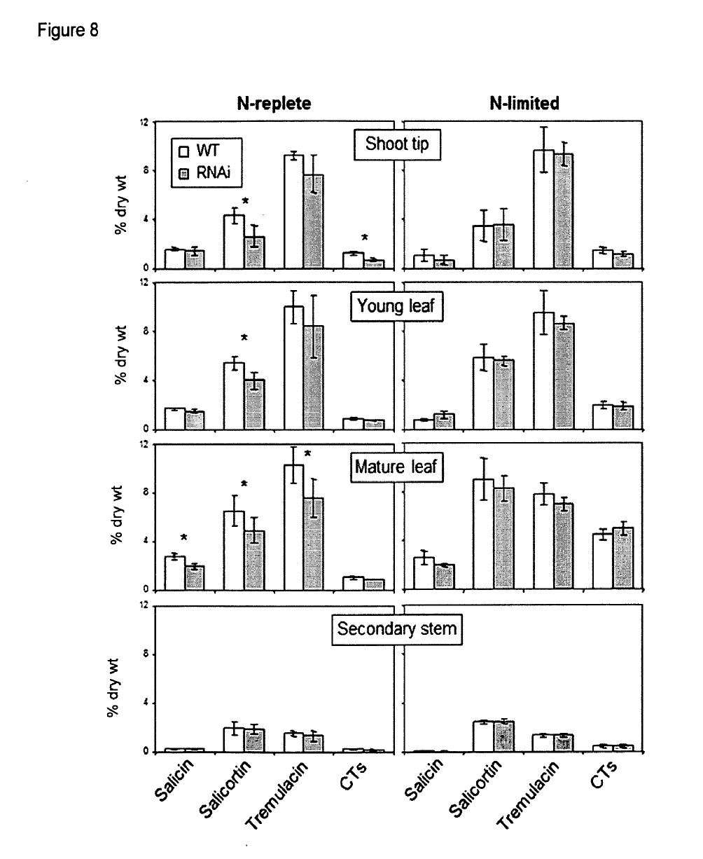

Concentrations of the higher-order PG salicortin and its precursor salicin were consistently lower, except for secondary stem internodes, of N-replete transgenic plants (FIG. 8). PGs were restored to WT levels in N-limited plants (FIG. 8). CT levels in shoot organs were low, compared to PGs, in all plants and were little affected by SUT4 suppression in N-replete plants (FIG. 8). CT levels increased several-fold with reduced N-status in both WT and transgenics, but there was no difference in response magnitude.

PtaSUT4 Supports Sucrose Efflux from Source Leaves

Several lines of evidence suggest that PtaSUT4 mediates tonoplast trafficking of sucrose which modulates efflux of sucrose from source leaves in Populus. First, PtaSUT4 was localized to the tonoplast, and exhibited an active sucrose transporter rather than a passive sucrose facilitator function. Second, transcript level of PtaSUT4 in source leaves was nearly two orders of magnitude greater than that of the Group II PtaSUT genes. In dicot annuals, the predominantly expressed SUT genes in source leaves are the Group II members, and Group IV genes are very wealdy expressed and found primarily in sink leaves (Chincinska et al., (2008) Plant Physiol., 146, 515-528, Endler et al., (2006) Plant Physiol., 141, 196-207, Weise et al., (2000) Plant Cell, 12, 1345-1355). Furthermore, in situ hybridization showed that PtaSUT4 transcripts in source leaves were not limited to minor vein traces as would be expected in apoplastic phloem loading species (Schmitt et al., (2008) Plant Physiol., 148, 187-199). Its expression throughout the spongy and lower palisade mesophyll cells is consistent with a role in symplastic transfer. Mesophyll expression has also been reported for Group IV SUT genes in two apoplastic loading species, Arabidopsis and barley, but at lower levels than the Group II members (Endler et al., (2006) Plant Physiol., 141, 196-207). Based on its tonoplast localization and its transcript abundance and distribution in source leaf lamina, PtaSUT4 could facilitate symplastic loading of the minor phloem companion cells by modulating cytosolic sucrose concentrations in the nearby mesophyll.

RNAi reduction of PtaSUT4 resulted in significant increases in source leaf sucrose levels, accompanied by higher starch levels compared to WT. This is certainly consistent with the supposition that photoassimilate export was reduced; but, at the same time, hexose levels decreased (FIG. 7 and Table 4). Therefore, the buildup of sucrose in the transgenic source leaves was probably due to reduced hydrolysis as well as reduced export. Additional data support the occurrence of both. First, changes in plant N-status affected vacuolar hydrolysis and sucrose accrual in a manner consistent with a coupling of those processes. The capacity for vacuolar hydrolysis was presumably reduced in source leaves of all N-limited plants, as VIN gene expression was greatly reduced (FIG. 6c,d). This coupled with impaired PtaSUT4 function led to a more significant sucrose buildup in the source leaves of N-limited transgenics, five times the differential observed between N-replete WT and transgenic plants. Second, support for reduced sucrose efflux is implicit based on the sucrose reductions observed in importing YL and ST organs of the transgenics (FIG. 7). In N-limited plants, the decrease in labile carbon in transgenic YL and ST did not reflect a utilization or dilution effect because early leaf expansion was slower than in WT (FIG. 11). If SUT4 can regulate vacuolar efflux, then SUT4 might contribute to phloem loading in a way envisaged for Salix (Turgeon and Medville, (1998) Proc. Natl. Acad. Sci. USA, 95, 12055-12060), whereby there would be a timely maintenance of high cytosolic concentrations of sucrose in mesophyll cells by active sucrose efflux from the vacuole. Vacuoles can serve as a daytime sucrose reservoir in plants (Gerhardt et al., (1987) Plant Physiol., 83, 399-407), and a SUT activity consistent with vacuolar efflux has already been demonstrated in vitro for the Group IV lotus LjSUT4 (Reinderset al., (2008) Plant Mol. Biol., 68, 289-299). The timing of vacuolar efflux would presumably be coordinated with sink demand and the supply of source leaf photoassimilate. Sucrose efflux from source leaves is highly regulated and varies during the light period, partly in relation to the synthesis and utilization of other carbohydrate reserves like starch (Cheikh and Brenner, (1992) Plant Physiol., 100, 1230-1237, Servaites et al., (1989) Plant Physiol., 90, 1168-1174, Sicher et al., (1984) Plant Physiol., 76, 165-169). The expression of SUT genes encoding plasmalemma proteins also varies during the photoperiod and as a function of changes in sink demand (Chincinska et al., (2008) Plant Physiol., 146, 515-528, Kuhn, (2003) Plant Biol., 5, 215-232, Kuhn et al., (1997) Science, 275, 1298-1300, Vaughn et al., (2002) Proc. Natl. Acad. Sci. USA, 99, 10876-10880). A ramification of the present findings is that PtaSUT4-mediated sucrose efflux from vacuoles may be a predominating feature of sucrose export from source leaves of Populus. However, the regulation of tonoplast SUT in relation to other carbohydrates, the photoperiod and sink-demand remains to be investigated. Based on microarray results, SUT4 transcript abundance in leaves of Populus is lower during the middle of the day than it is during the dark period (Wilkins et al., (2009) Plant J, 60, 703-715). Assuming that the RNAi effect on SUT4 transcript levels was consistent throughout the diel, we suggest that a diurnal rhythm of SUT4 expression was preserved in the transgenics, albeit at a lower transcript level. This is consistent with the observation that source leaf sucrose contents were reduced by a similar extent at the end of the photoperiod and at the end of the dark period in the transgenic plants (FIG. 13).

### PtSUT4 is Necessary for Efficient Sucrose Transport Through the Stem

The efficiency of long-distance sucrose transport in wood-forming perennials may benefit from an abundantly expressed tonoplastic SUT. We found that when PtaSUT4 was down-regulated, sucrose increased to a much greater extent in stem phloem (25%) and xylem (28%) than in source leaves (13%). It is likely based on sucrose:glucose ratio that only a small proportion of the phloem tissue analyzed comprised transport cells (Dafoe et al., (2009) J. Proteome Res., 8, 2341-2350). Therefore, the large glucose decrease in transgenic phloem appears to reflect an alteration to sugar partitioning in lateral phloem sinks outside of the transport cells. A significant fraction of transport sucrose escapes from the sieve tubes into the apoplastic space (Van Bel, (2003) Plant Physiol., 131, 1509-1510), and part of this fraction is recovered by Group II SUT proteins and reloaded into the companion cells for continued transport (Srivastava et al., (2008) PlantPhysiol., 148, 200-211). Sucrose recovery by adjacent lateral sinks could occur via endocytosis (Etxeberria et al., (2005) Plant Cell Physiol., 46, 474-481), although that has only been demonstrated for cultured sycamore cells, or via SUT proteins that sense intracellular osmotic potentials and sucrose levels (Zhou et al., (2009) J. Exp. Bot., 60, 71-85). The recovery is affected by the ability of lateral sinks to compete for apoplastic solutes as well as by thermodynamic characteristics of the transport stream (Hafke et al., (2005) Plant Physiol., 138, 1527-1537). Because PtaSUT4 was strongly expressed throughout the inner phloem (FIG. 3), its down-regulation would presumably affect the compartmentation and metabolic partitioning of sucrose after its uptake by numerous phloem sinks. Interestingly, sucrose that is taken up endocytotically by sycamore cells accumulates in the vacuole (Etxeberria et al., (2005) Plant Cell Physiol., 46, 474-481). Transcript abundance of VIN2 is very low in phloem (FIG. 12, inset), and in combination with SUT4 down-regulation, this would be expected to result in decreased hydrolysis of sucrose, decreased glucose levels and enhanced sucrose accrual as observed. The perturbations to sucrose compartmentation or their possible connection with the observed pooling of sucrose may imply that net transport of sucrose through the phloem was altered, but this remains to be investigated.

A significant sucrose enhancement was also observed in xylem of transgenic relative to WT plants. Hexose levels were not affected, and starch levels were negligible. As in source leaves, the sucrose enhancement was larger in the xylem of N-limited plants with sharply reduced stem VIN gene expression (FIGS. 6 and 7). Based on our sucrose estimates, and assuming a wood bulk density in young Populus shoots of 0.37 μm cm-3 (DeBell et al., (2002) Wood Fiber Sci., 34, 529-539), the sucrose level in xylem of N-limited RNAi plants was comparable to seasonal xylem sap maxima reported for Salix and Juglans species, in the range of 125-175 mM (Decourteix et al., (2006) Plant Cell Environ., 29, 36-47, Sauter, (1983) Zeitschrift Fur Pflanzenphysiologie, 111, 429-440). This level was more than twice the level observed in WT xylem (FIG. 7). Seasonally-regulated apoplastic and symplastic exchange of sucrose between xylem vessels and vessel-associated parenchyma occurs in tree stems (Alves et al., (2001) J. Plant Physiol., 158, 1263-1271, Ameglio et al., (2004) Tree Physiol., 24, 785-793, Sauter, (1983) Zeitschrift Fur Pflanzenphysiologie, 111, 429-440), and subcellular compartmentation of sucrose is thought to be involved in the control of that exchange (Sauter, (1988) J. Plant Physiol., 132, 608-612). Group II SUT proteins that mediate apoplastic sucrose transport participate in regulating this process (Decourteix et al., (2008) Tree Physiol., 28, 215-224, Decourteix et al., (2006) Plant Cell Environ., 29, 36-47). By down-regulating the tonoplast PtaSUT4, we altered the ratio of the Group II PtaSUT3 transcripts, associated with apoplastic transport, to the Group IV PtaSUT4, associated with intracellular transport. As a result, the reduction of PtaSUT4 activity could affect sucrose utilization and transport in a variety of ways depending on how directly PtaSUT3 and PtaSUT4 proteins compete for transport sucrose.

PtaSUT4 Modulates Expression of Genes that Control Carbon Partitioning

The RNAi effect on transcript levels of well-expressed SUS, N1N, SPS and PAL genes was N-status dependent, especially in source leaves. In contrast to the situation in N-replete plants, SUS genes were strongly up-regulated in source leaves of the transgenics during N-limitation. The discrepancy was not surprising, considering that the dynamic of sucrose compartmentation and utilization in source leaf cells probably differs between N-replete and N-limited plants. SUS1 and SUS2 transcripts are normally abundant in stems (FIG. 12), where sucrose synthase is instrumental in cellulose deposition during secondary cell wall development (Andersson-Gunneras et al., (2006) Plant J., 45, 144-165, Coleman et al., (2009) Proc. Natl. Acad. Sci. USA, 106, 13118-13123, Delmer and Amor, (1995) Plant Cell, 7, 987-1000). Because cell wall development is stimulated in N-limited Populus (Cooke et al., (2003) Plant Cell Environ., 26, 757-770), one model supported by the current findings is that leaf vascularization and cell wall thickening increased as N deficit became more severe in all plants, and this process advanced more rapidly in source leaves of the transgenics.

PAL2 transcripts are normally limited to vascular tissues of Populus, and comprise a minor fraction of overall PAL expression in leaves (Kao et al., (2002) Plant Physiol., 130, 796-807). In the present study, while PAL1 transcript level decreased in source leaves of N-limited plants, PAL2 transcript abundance increased (FIG. 6). The N stress-induced PAL2 expression was much more substantial in the source leaves of transgenics (>4-fold) than WT (2-fold). The PAL2 expression patterns therefore paralleled those of SUS2, and supported the idea that the PtaSUT4-mediated metabolic processes included cell wall biosynthesis. Interestingly, PG levels also increased more in leaves of transgenic plants during N-stress, while CT levels increased similarly in both genotypes (FIG. 8). PGs comprise both glucose and phenylpropanoid moieties (Lindroth and Hwang, (1996) Biochem. Syst. Ecol., 24, 357-364, Pierpoint, (1994) Adv. Bot. Res., 20, 163-235). As in the case of cellulose biosynthesis, PG biosynthesis may depend upon a SUS-mediated supply of UDP-glucose, a supply that was reduced in N-replete transgenics with reduced SUS expression, and restored in N-limited transgenics. CT, on the other hand, is biosynthesized from acetyl and phenylpropanoid moieties (Heller and Hahlbrock, (1980) Arch. Biochem. Biophys., 200, 617-619) and may be less directly affected by changes in sugar partitioning.

SPS activity is thought to be an important determinant of triose-phosphate utilization for sucrose biosynthesis at the expense of starch (Smith and Stitt, (2007) Plant Cell Environ., 30, 1126-1149). The source leaf patterns of SPS6 expression and starch accrual we observed were consistent with such a relationship. SPS6 transcript and starch levels differed little between N-replete and N-limited transgenic plants, but SPS6 transcript and starch levels trended in opposite directions in WT leaves depending on N-status. Together, the gene expression and metabolite findings reinforce the idea that there was a fundamental effect of PtaSUT4 down-regulation on N-responsiveness of carbon partitioning between primary and secondary (phenylpropanoid) metabolism in Populus. The reduced N-status led to enhanced secondary metabolism at the expense of transient starch storage in transgenic source leaves compared to WT source leaves. The opposite occurred in N-replete plants where transient starch storage was higher and PG levels lower in the transgenics. The gene expression relationships observed in the exporting source leaf differed markedly from those observed in importing YL and terminal sink ST leaves engaged in growth. Overall, PtaSUT4 appears to have a role in coordinating the metabolic fate of sucrose, along with its efflux from source leaves and distribution among lateral and terminal sinks with plant N-status.

In summary, PtaSUT4 can function along with vacuolar invertases in the phloem, xylem and source leaf to orchestrate subcellular sucrose compartmentation and long-distance transport in Populus. PtaSUT4 appears to modulate both sucrose export and its utilization by lateral and terminal sinks in concert with plant N-status. Impairment of SUT4 function altered biomass distribution between leaves and stems, and suggested a link between such biomass partitioning and vacuolar trafficking of sucrose in source leaves, and along the transport path in stems.

### Example 2

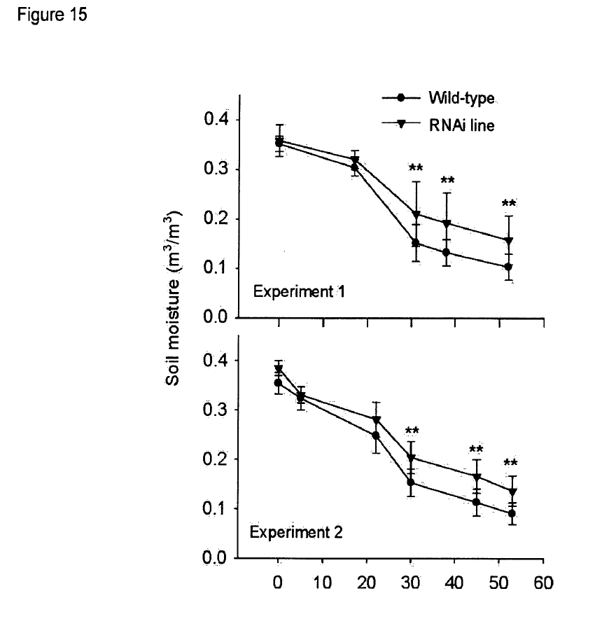

The tonoplast sucrose transporter, PtaSUT4, transports sucrose intracellularly from the vacuole to the cytosol. When PtaSUT4 is down-regulated in Populus (Example 1), there are tissue-level changes in the concentrations of glucose and fructose, which are products of sucrose catabolism that also happen to be important osmolytes. We suggest that altered sucrose intracellular compartmentalization resulted in changes in osmolyte homeostasis that have the potential to alter plant water status and perhaps, utilization and uptake. Indeed, we found that water use efficiency (WUE) was increased in the SUT4-silenced transgenic Populus. Water uptake, as measured by soil water loss, was found to be lower in the SUT4-RNAi plants than in wild type controls in several replicate experiments (FIG. 15). Despite the lower rate of water uptake, shoot (woody) and leaf biomass were not reduced in the SUT4-RNAi plants. On the basis of increased shoot dry biomass produced per unit of water taken up, this constitutes improved WUE by the transgenic plants.

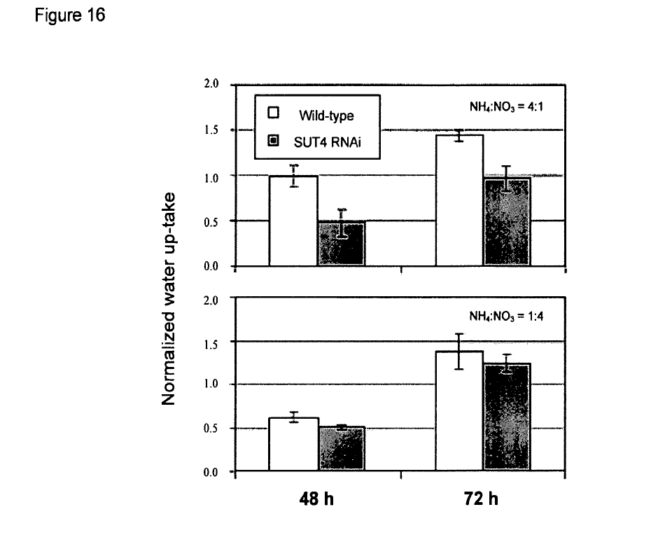

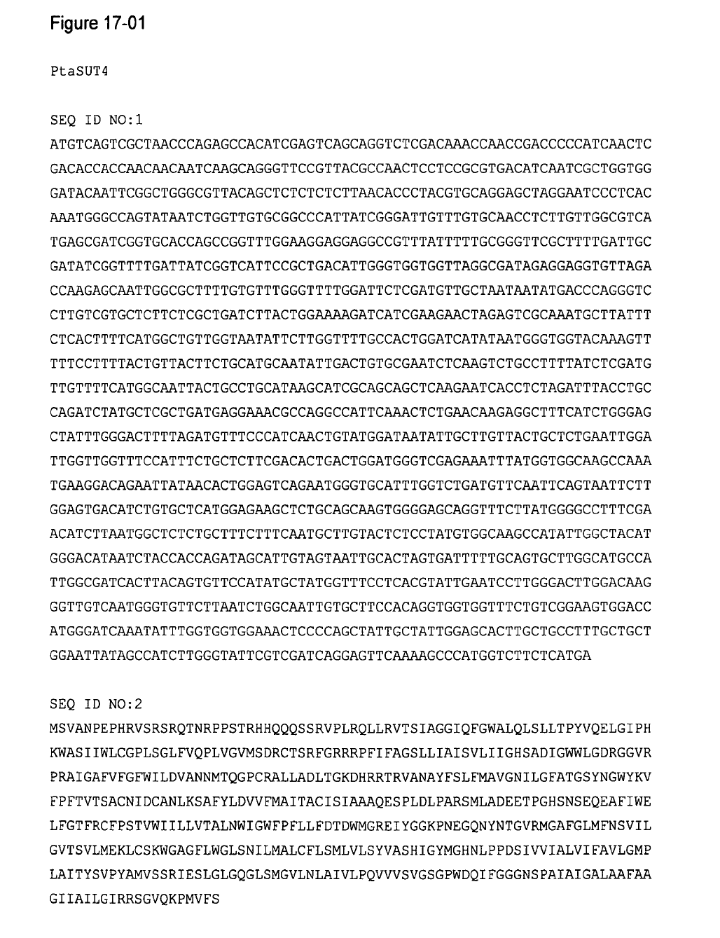

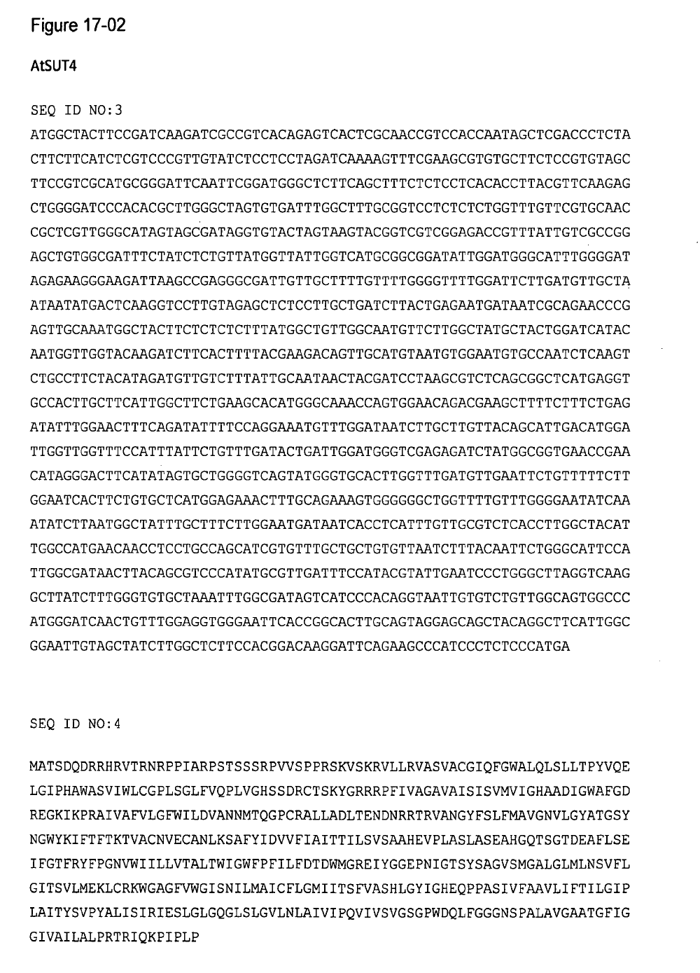

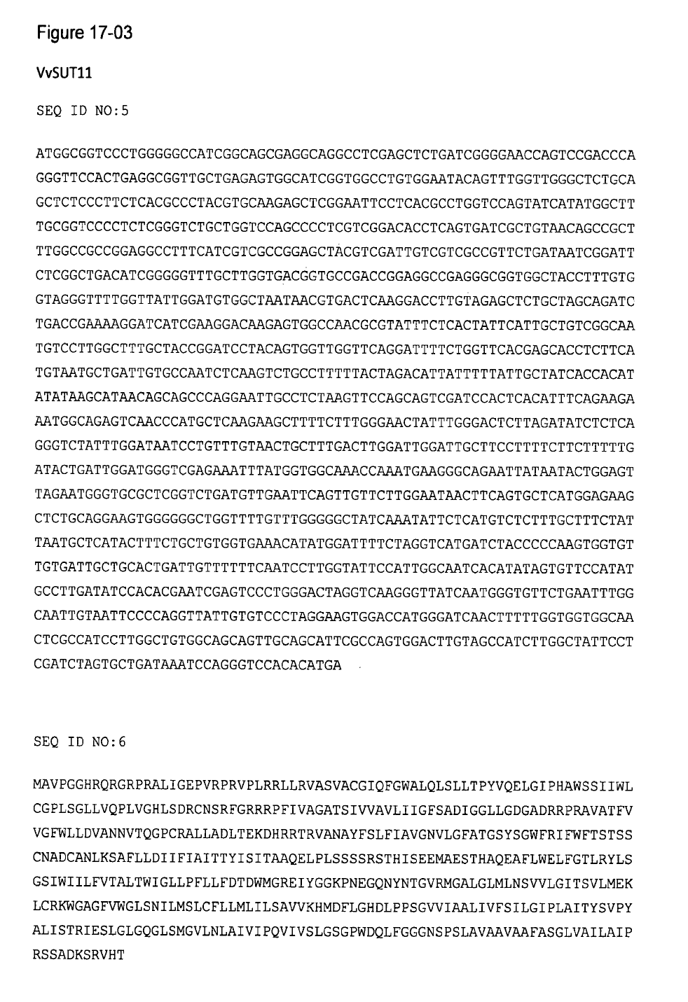

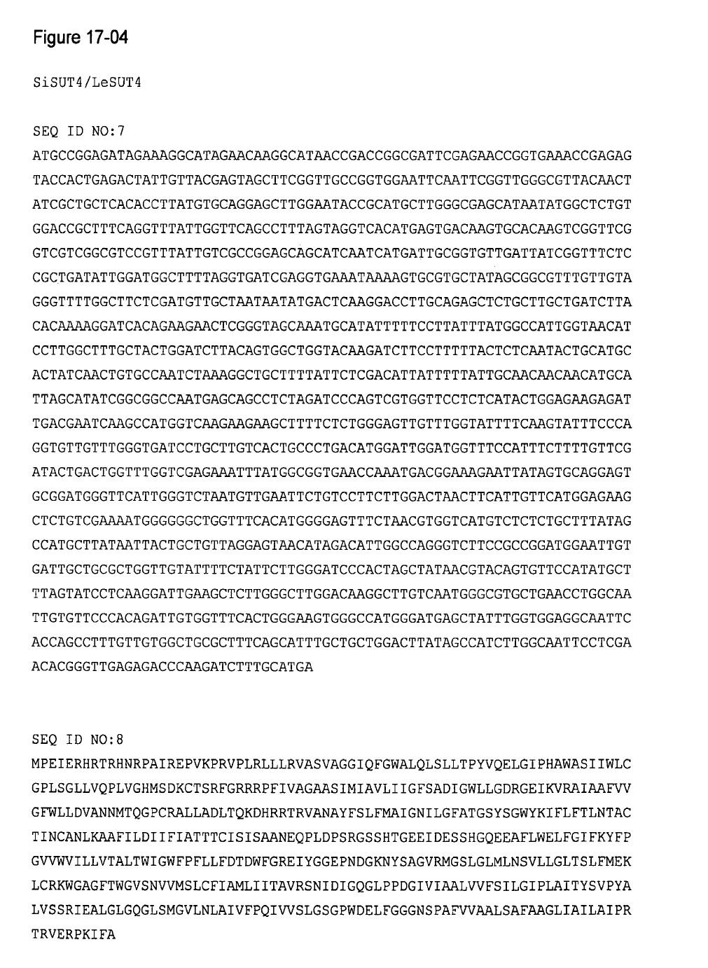

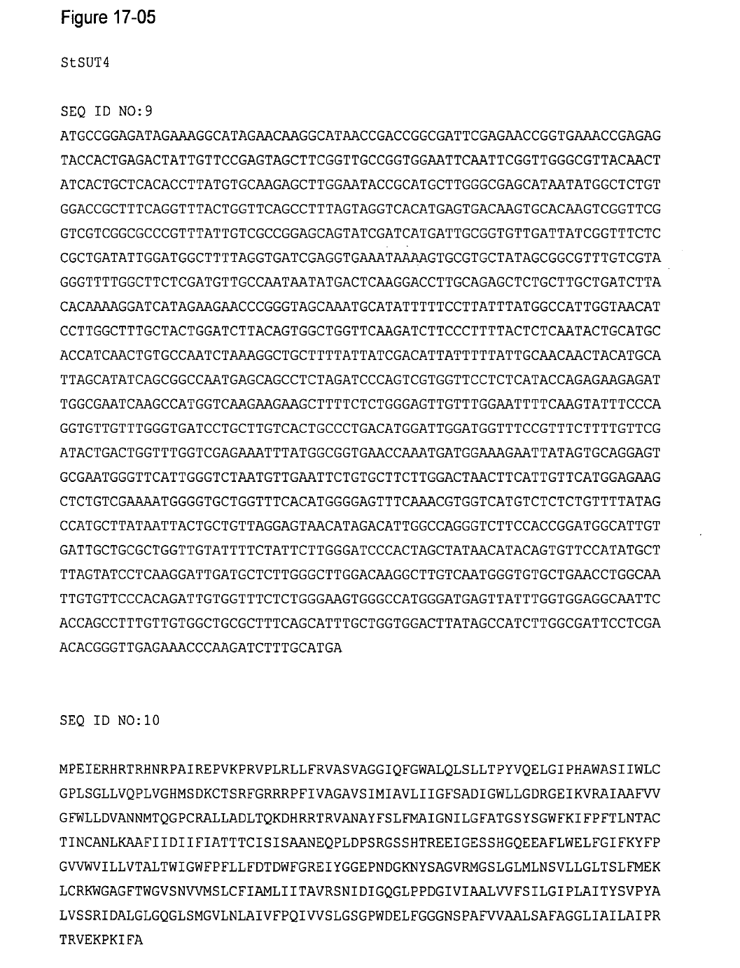

Example 1 presents evidence that nitrogen sensing or utilization might be altered in the transgenic plants. That conclusion was based on changes in the abundance of nitrogen responsive metabolites following manipulation of plant N-status by hydroponic nitrogen nutrient treatments. We now have evidence that the WUE increase in the transgenic plants may be conditioned by nitrogen source. When plants were grown with nutrient solution containing nitrogen as ammonium and nitrate in a ratio of 4:1, the water uptake differential between transgenics and wild type plants was relatively high (FIG. 16). When plants were fertilized with nutrient solution containing nitrogen as ammonium:nitrate in a ratio of 1:4, water uptake by the wild type plants was still higher, but by a smaller margin (FIG. 16). Under both nitrogen regimes, the higher WUE in the transgenics was due to lower rates of water uptake compared to the wild type controls, with no reduction in shoot dry biomass. Nitrate and ammonium nitrogen are the two most utilized forms of soil nitrogen by higher plants including Populus. The ratio between the two depends on seasonal, climatic, hydrologic and compositional factors that impact soil moisture content and soil microbial activity. Tailored expression of PtaSUT4 by use of the appropriate promoters could conceivably lead to improved WUE by optimizing PtaSUT4 gene expression according to soil water status and nitrogen composition and availability.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method for using a transgenic plant comprising:
growing a transgenic plant in drought conditions, wherein the plant comprises decreased expression of a SUT polypeptide compared to a control plant.

2. The method of claim 1 wherein the transgenic plant has a phenotype of increased growth, increased formation of woody tissue, increased drought tolerance, increased water use efficiency, increased nitrogen utilization efficiency, or a combination thereof, compared to the control plant.

3. The method of claim 1 wherein the SUT polypeptide has at least 80% amino acid sequence identity to SEQ ID NO:2.

4. The method of claim 1 wherein the expression of the SUT polypeptide is decreased in the transgenic plant by at least 10% when grown in drought conditions compared to the transgenic plant not grown in the stress condition.

5. The method of claim 1 wherein the plant is a dicot.

6. The method of claim 1 wherein the transgenic plant is a woody plant.

7. The method of claim 6 wherein the transgenic plant is a member of the genus Populus.

8. A method for maintaining stem growth during exposure to a stress condition comprising growing a transgenic plant under stress conditions, wherein the transgenic plant comprises decreased expression of a coding region encoding a SUT polypeptide compared to a control plant.

9. The method of claim 8 wherein the expression of the SUT polypeptide is decreased in the transgenic plant by at least 10% when grown in a stress condition compared to the transgenic plant not grown in the stress condition.

10. The method of claim 8 wherein the plant is a dicot.

11. The method of claim 8 wherein the transgenic plant is a woody plant.

12. The method of claim 11 wherein the transgenic plant is a member of the genus Populus.

13. The method of claim 8 wherein the stress condition is selected from drought, heat, salt, or the combination thereof.

14. A method using a plant having increased water use efficiency comprising growing a transgenic plant under conditions of decreased water availability, wherein the transgenic plant comprises decreased expression of a coding region encoding a SUT polypeptide compared to a control plant.

15. A method for using a transgenic plant, the method comprising processing a transgenic plant to result in pulp, wherein the transgenic plant comprises decreased expression of a coding region encoding a SUT polypeptide compared to a control plant.

16. The method of claim 15 wherein the processing comprises a physical pretreatment, a chemical pretreatment, or a combination thereof.

17. The method of claim 15 further comprising hydrolyzing the pulp.

18. The method of claim 15 further comprising contacting the pulp with a microbe under conditions suitable for fermentation of the pulp.

19. The method of claim 15 further comprising obtaining a metabolic product.

20. The method of claim 19 wherein the metabolic product comprises ethanol.

21. The pulp of claim 15.

22. A transgenic plant comprising an exogenous polynucleotide, wherein expression of the exogenous polynucleotide inhibits expression of a SUT polypeptide, and wherein the exogenous polynucleotide is operably linked to a promoter responsive to a stress condition.

23. The transgenic plant of claim 22 wherein the exogenous polynucleotide comprises at least 19 nucleotides that are substantially identical or substantially complementary to SEQ ID NO:1.

24. The transgenic plant of claim 22 wherein the transgenic plant is a dicot plant.

25. A part of the transgenic plant of claim 22 wherein the part is chosen from a leaf, a stem, a flower, an ovary, a fruit, a seed, and a callus.

26. The progeny of the transgenic plant of claim 22.

27. The progeny of claim 26 wherein the progeny is a hybrid plant.

28. A wood obtained from the transgenic plant of claim 22.

29. A wood pulp obtained from the transgenic plant of claim 22.
